(19) United States
(12) Patent Application Publication
(10) Pub. No.: US 2019/0233522 A1
(43) Pub. Date: Aug. 1, 2019

(54) NEW DOSAGE REGIMENS FOR ANTIBODY DRUG CONJUGATES BASED ON ANTI-AXL ANTIBODIES

(71) Applicant: GENMAB A/S, Copenhagen V (DK)

(72) Inventors: Ulf FORSSMANN, Hannover (DE); Steen LISBY, Frederiksberg (DK); Nedjad LOSIC, Limhamn (SE)

(21) Appl. No.: 16/316,000

(22) PCT Filed: Jul. 7, 2017
(86) PCT No.: PCT/EP2017/067101

§ 371 Date: Jan. 7, 2019

Related U.S. Application Data
(60) Provisional application No. 62/410,984, filed on Oct. 21, 2016.

(30) Foreign Application Priority Data
Jul. 8, 2016 (EP) PCT/EP2016/066353

(51) Int. Cl. C07K 16/28; A61K 47/68; A61P 35/00

(52) U.S. Cl. CPC C07K 16/2863; A61K 47/6803; A61P 35/00; A61K 47/6851; A61K 47/6849

(57) ABSTRACT

An antibody-drug conjugate (ADC) based on an antibody binding to human AXL and pharmaceutical compositions comprising the ADC for use in the treatment of a cancer comprising administering to a subject a weekly dose of from about 0.45 mg/kg to about 2.0 mg/kg of the ADC once a week for three consecutive weeks followed by a one week resting period without any administration of the ADC so that each cycle time is 28 days including the resting period.

### FIELD OF THE INVENTION

The present invention relates, inter alia, to antibody-drug conjugates (ADCs) based on anti-AXL antibodies and their use in treating a cancer. In particular, the present invention relates to dosage regimens for such ADCs comprising administering to a subject a weekly dose of from about 0.45 mg/kg to about 2.0 mg/kg of the ADC.

### BACKGROUND OF THE INVENTION

AXL is a 104-140 kDa transmembrane protein which belongs to the TAM subfamily of mammalian Receptor Tyrosine Kinases (RTKs). AXL is believed to be involved in several cellular functions, including growth, migration, aggregation and anti-inflammation in multiple cell types, and is weakly expressed on normal cells, predominantly observed in fibroblasts, myeloid progenitor cells, macrophages, neural tissues, cardiac and skeletal muscle. AXL can be activated upon binding of its ligand, the vitamin K-dependent growth arrest-specific factor 6 (Gas6), leading to AXL dimerization, autophosphorylation and subsequent activation of intracellular signalling pathways.

Upregulation of AXL has been reported in a variety of cancers, including lung cancer, ovarian cancer, endometrial cancer, cervical cancer, thyroid cancer and melanoma (Paccez et al., 2014; Sun et al., 2003). AXL expression in cancer cells has been associated with tumor cell motility, invasion, migration, and epithelial-to-mesenchymal transition (EMT) (Linger et al., 2010).

In addition, several types of cancer associated with resistance to certain drugs have been found to show enhanced or de novo AXL protein. This has been reported in, for example, non-small cell lung cancer (NSCLC) resistant to erlotinib and crizotinib (Zhang et al., 2012; Wilson et al., 2014; Kim et al., 2013) and in melanoma resistant to vemurafenib or selumetinib (Müller et al., 2014; Konieczkowski et al., 2014).

ADCs based on anti-AXL antibodies for use in treating cancer have been described, e.g., in WO 2016/005593 A1 (Genmab), WO 2017/009258 (Genmab) and WO 2014/174111 (Pierre Fabré Medicament and Spirogen Sarl). The optimal dose regimen for such an ADC for human subjects should be based on a balance between efficacy and safety, both of which are influenced by the dose and the frequency of administration. For example, US 2011/0268751 A1 (Seattle Genetics) describes a weekly dosage regimen for an anti-CD30 antibody-drug conjugate in treating hematological cancers such as Hodgkin's lymphoma and anaplastic large cell lymphoma. As a target for ADC therapy, however, each cell surface antigen offers unique characteristics in terms of tissue distribution, internalization into the cell and other properties. Further, the accessibility of the target antigen differs between cancer types, e.g., between hematological cancers and solid cancers.

Accordingly, there remains an unmet medical need for patients suffering from any of the above mentioned cancers and other cancers associated with AXL expression, in particular with respect to dosage regimens for an ADC based on an antibody binding human AXL.

### OBJECT OF THE INVENTION

It is an object of the present invention to provide methods for treating such cancers. It is a further object of the present invention to provide a dosing regimen for ADCs based on anti-AXL antibodies for use in methods of treating such cancers. It is a further object of the present invention to provide a safe and efficacious new dosing regimen for ADCs based on specific anti-AXL antibodies.

### SUMMARY OF THE INVENTION

The present inventors have developed a weekly dosing regimen for three consecutive weeks of ADCs based on anti-AXL antibodies (herein also referred to as “AXL-ADCs”), which provides an efficacious therapeutic regimen and has an acceptable tolerability profile despite the frequent dosing. Accordingly, the present invention relates to an AXL-ADC for use in the treatment of cancers wherein the AXL-ADC is administered to a subject in need thereof in cycles of once a week for three consecutive weeks followed by a one week rest period.

The invention further relates to a pharmaceutical composition comprising an ADC of the formula:

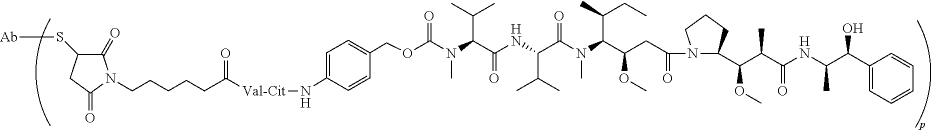

or a pharmaceutically acceptable salt thereof and a pharmaceutical acceptable carrier, wherein the mAb is an anti-AXL antibody, S is a sulfur atom of the antibody, p is from 3-5, for use in a method of treating a cancer wherein the pharmaceutical composition is administered to a subject in need thereof in cycles of once a week for three consecutive weeks followed by a one week rest period.

### BRIEF DESCRIPTION OF THE DRAWINGS

FIG. 1: Overview of the Target Mediated Drug Disposition (TMDD) model, showing two-compartmental (A) and target-mediated clearance (B) models.

FIG. 2: Standard Goodness-of-fit (GOF) plots for the ADC/IgG cynomolgus monkey model (A-F).

FIG. 3: Standard GOF plots for the cynomolgus monkey MMAE model (A-F).

FIG. 4: Predicted Human ADC (A) and MMAE (B) levels for 0.3 mg/kg AXL-ADC, dosing once every 3 weeks (1Q3W).

FIG. 5: Predicted Human ADC (A) and MMAE (B) levels for 0.6 mg/kg, 1Q3W.

FIG. 6: Predicted Human ADC (A) and MMAE (B) levels for 1 mg/kg, 1Q3W.

FIG. 7: Predicted Human ADC (A) and MMAE (B) levels for 1.5 mg/kg, 1Q3W.

FIG. 8: Predicted Human ADC (A) and MMAE (B) levels for 2 mg/kg, 1Q3W.

FIG. 9: Predicted Human ADC (A) and MMAE (B) levels for 2.4 mg/kg, 1Q3W.

FIG. 10: Predicted Human ADC (A) and MMAE (B) levels for 0.6 mg/kg, after weekly dosing for 3 weeks followed by one treatment-free week (3Q4W).

FIG. 11: Predicted Human ADC (B) and MMAE (B) levels for 0.8 mg/kg, 3Q4W.

FIG. 12: Predicted Human ADC (B) and MMAE (B) levels for 1 mg/kg, 3Q4W.

FIG. 13: Predicted Human ADC (A) and MMAE (B) levels for 1.2 mg/kg, 3Q4W.

FIG. 14: Predicted Human ADC (A) and MMAE (B) levels for 1.4 mg/kg, 3Q4W.

FIG. 15: Induction of cytotoxicity by ADCs in LCLC-103H cells was determined as described in Example 2.

FIG. 16: Induction of cytotoxicity by AXL-ADCs in A431 cells (A) and MDA-MB231 cells (B) was determined as described in Example 2.

FIG. 17: Anti-tumor activity by MMAE-conjugated AXL antibodies in a therapeutic LCLC-103H xenograft model as described in Example 3.

FIG. 18: (A) Average tumor size after therapeutic treatment with AXL-ADCs the PAXF1657 model. An unconjugated AXL Humab (C) and an untargeted ADC (D) do not show anti-tumor activity, indicating that the therapeutic capacity of AXL-ADCs was dependent on the cytotoxic activity of MMAE and on target binding, error bars represent S.E.M.

FIG. 19: Anti-tumor activity of MMAE-conjugated AXL antibodies in a therapeutic A431 xenograft model, that produces high levels of endogeneous Gas6, as described in Example 5. Panels A and B show results from 2 independent experiments.

FIG. 20: Anti-tumor activity of MMAE-conjugated AXL antibodies in a therapeutic LCLC-103H xenograft model, that expresses low levels of endogenous Gas6, as described in Example 5. Panels A and B show results from 2 independent experiments.

FIG. 21. AXL staining in thyroid, esophageal, ovarian, breast, lung, pancreatic, cervical and endometrial cancer. The average AXL staining intensity (OD) of AXL-positive cells is plotted on the X-axis, and the percentage of AXL-positive tumor cells is plotted on the Y-axis. Each dot represents a tumor core, derived from an individual patent.

FIG. 22. (A) Average tumor size after therapeutic treatment with IgG1-AXL-107-vcMMAE in the esophageal cancer PDX model ES0195. IgG1-b12 and IgG1-b12-MMAE were included as isotype control antibody and isotype control ADC, respectively. (B) Tumor size in individual mice on day 32 after injection of MDA-MB-231-luc D3H2LN tumor cells in the mammary fat pads of female SCID mice. * p<0.05; ** p<0.0001

FIG. 23. Therapeutic effect of AXL-ADCs in a patient-derived cervical cancer xenograft model. (A) Average tumor size after therapeutic treatment with IgG1-AXL-183-vcMMAE or IgG1-AXL-726-vcMMAE in the cervical cancer PDX model CEXF 773. IgG1-b12 and IgG1-b12-MMAE were included as isotype control antibody and isotype control ADC, respectively. (B) Tumor size in individual mice on day 28 after initiation of treatment in the cervical cancer PDX model CEXF 773. * p<0.001.

FIG. 24. Therapeutic activity of AXL-ADCs in an orthotopic breast cancer xenograft model. (A) Average tumor size after therapeutic treatment with IgG1-AXL-183-vcMMAE or IgG1-AXL-726-vcMMAE in an orthotopic MDA-MB-231-luc D3H2LN xenograft model. IgG1-b12 and IgG1-b12-MMAE were included as isotype control antibody and isotype control ADC, respectively. (B) Tumor size in individual mice on day 32 after injection of MDA-MB-231-luc D3H2LN tumor cells in the mammary fat pads of female SCID mice. * p<0.001.

FIG. 25. Improved anti-tumor efficacy of IgG1-AXL-107-vcMMAE in an erlotinib-resistant NSCLC patient-derived xenograft (PDX) model in combination with erlotinib. Average tumor size after therapeutic treatment with IgG1-AXL-107-vcMMAE, erlotinib, or erlotinib in combination with IgG1-AXL-107-vcMMAE in the NSCLC PDX model LU2511. IgG1-b12 and IgG1-b12-MMAE were included as isotype control antibody and isotype control ADC, respectively. *, p<0.05; **, p<0.01; ns, not significant (one-way ANOVA test).

FIG. 26. Anti-tumor efficacy of IgG1-AXL-107-vcMMAE in the erlotinib-resistant LU0858 NSCLC patient-derived xenograft (PDX) model. Average tumor size after therapeutic treatment with IgG1-AXL-107-vcMMAE, erlotinib, or erlotinib in combination with IgG1-AXL-107-vcMMAE is shown (A). IgG1-b12 and IgG1-b12-MMAE were included as isotype control antibody and isotype control ADC, respectively. Mean tumor size and SEM of each group per time point and tumor size per individual mouse per group on day 11 (B) and day 21 (C) are shown. *, p<0.05; **, p<0.01; ns, not significant (Mann-Whitney test).

FIG. 27. Anti-tumor efficacy of IgG1-AXL-107-vcMMAE in the erlotinib-resistant LU1868 NSCLC patient-derived xenograft (PDX) model. Average tumor size after therapeutic treatment with IgG1-AXL-107-vcMMAE, erlotinib, or erlotinib in combination with IgG1-AXL-107-vcMMAE is shown (A). IgG1-b12 and IgG1-b12-MMAE were included as isotype control antibody and isotype control ADC, respectively. Mean tumor size and SEM of each group per time point and tumor size per individual mouse per group on day 21 (B), day 28 (C) and day 31 (D) are shown. *, p<0.05; **, p<0.01; ns, not significant (Mann-Whitney test).

FIG. 28. Anti-tumor efficacy of IgG1-AXL-107-vcMMAE in the erlotinib-resistant LXFA 526 NSCLC patient-derived xenograft (PDX) model. (A) Average tumor size after therapeutic treatment with IgG1-AXL-107-vcMMAE, erlotinib, or erlotinib in combination with IgG1-AXL-107-vcMMAE is shown. (B) Mean tumor size and SEM of each group per time point and tumor size per individual mouse per group on day 23. *, p<0.05; **, p<0.01; ns, not significant (Mann-Whitney test).

FIG. 29. Anti-tumor efficacy of IgG1-AXL-107-vcMMAE in the NSCLC patient-derived xenograft (PDX) model LXFA 677 (A) and LXFA 677_3 (C), which has acquired resistance to erlotinib. Average tumor size after therapeutic treatment with IgG1-AXL-107-vcMMAE, erlotinib, or erlotinib in combination with IgG1-AXL-107-vcMMAE is shown. (B, D) Mean tumor size and SEM of each group per time point and tumor size per individual mouse per group on day 21 of the LXFA 677 model (B) or on day 37 of the LXFA 677_3 model (D). *, p<0.05; **, p<0.01; ns, not significant (Mann-Whitney test).

FIG. 30. Anti-tumor efficacy of IgG1-AXL-107-vcMMAE in the cervical cancer PDX model CV1664. (A) Average tumor size after therapeutic treatment with IgG1-b12, IgG1-b12-vcMMAE, IgG1-AXL-107, IgG1-AXL-107-vcMMAE, or paclitaxel is shown. (B) Mean tumor size and SEM of each group per time point and tumor size per individual mouse per group on day 46 is shown. (C, D) Average tumor size in IgG1-AXL-107-vcMMAE—(C) or paclitaxel-treated (D) mice that were retreated with IgG1-AXL-107-vcMMAE is shown. *, p<0.05; **, p<0.01; ns, not significant (Mann-Whitney test).

### DETAILED DISCLOSURE OF THE INVENTION

As described herein, the present invention relates to AXL-specific ADCs (also referred to as “AXL-ADCs” or “anti-AXL antibody drug conjugates” herein) as defined in any aspect or embodiment herein, for use in treating cancers. In particular, a new dosage regimen for an AXL-ADC is provided. The dosage regimen provides an efficacious therapeutic regimen for treating cancer and has an acceptable tolerability and safety profiles, despite the frequent dosing.

Definitions

The term “AXL” or “Axl” as used herein, refers to the protein entitled AXL, which is also referred to as UFO or JTK11, a 894 amino acid protein with a molecular weight of 104-140 kDa that is part of the subfamily of mammalian TAM Receptor Tyrosine Kinases (RTKs). The molecular weight is variable due to potential differences in glycosylation of the protein. The AXL protein consists of two extracellular immunoglobulin-like (Ig-like) domains on the N-terminal end of the protein, two membrane-proximal extracellular fibronectin type III (FNIII) domains, a transmembrane domain and an intracellular kinase domain. AXL is activated upon binding of its ligand Gas6, by ligand-independent homophilic interactions between AXL extracellular domains, by autophosphorylation in presence of reactive oxygen species (Korshunov et al., 2012) or by transactivation through EGFR (Meyer et al., 2013), and is aberrantly expressed in several tumor types. In humans, the AXL protein is encoded by a nucleic acid sequence encoding the amino acid sequence shown in SEQ ID NO:130 (human AXL protein: Swissprot P30530). For cynomolgus AXL protein, see Genbank accession HB387229.1 (SEQ ID NO:147).

The term “Growth Arrest-Specific 6” or “Gas6” as used herein, refers to a 721 amino acid protein, with a molecular weight of 75-80 kDa, that functions as a ligand for the TAM family of receptors, including AXL. Gas6 is composed of an N-terminal region containing multiple gamma-carboxyglutamic acid residues (Gla), which are responsible for the specific interaction with the negatively charged phospholipid membrane. Although the Gla domain is not necessary for binding of Gas6 to AXL, it is required for activation of AXL. Gas6 may also be termed as the “ligand to AXL”.

When used herein in the context of an antibody and a Gas6 ligand or in the context of two or more antibodies, the term “competes with” or “cross-competes with” indicates that the antibody competes with the ligand or another antibody, e.g., a “reference” antibody in binding to an antigen, respectively. Example 2 of WO 2016/005593 A1 (Genmab) describes an example of how to test competition of an anti-AXL antibody with the AXL-ligand Gas6. Preferred reference antibodies for cross-competition between two antibodies are those comprising a binding region comprising the VH region and VL region of an antibody herein designated 107, 148, 733, 154, 171, 183, 613, 726, 140, 154-M103L, 172, 181, 183-N52Q, 187, 608-01, 610-01, 613-08, 620-06 or 726-M101L, as set forth in Table 2. A particularly preferred reference antibody is the antibody designated 107.

The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., ed., 2nd ed. Raven Press, N.Y. (1989). Within the structure of the immunoglobulin, the two heavy chains are inter-connected via disulfide bonds in the so-called “hinge region”. Equally to the heavy chains each light chain is typically comprised of several regions; a light chain variable region (abbreviated herein as VL region) and a light chain constant region. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences are defined according to IMGT (see Lefranc et al. (1999) and Brochet et al. (2008)).

The term “immunoglobulin heavy chain” or “heavy chain of an immunoglobulin” as used herein is intended to refer to one of the heavy chains of an immunoglobulin. A heavy chain is typically comprised of a heavy chain variable (abbreviated herein as VH) region and a heavy chain constant region (abbreviated herein as CH) which defines the isotype of the immunoglobulin. The heavy chain constant region typically is comprised of three domains, CH1, CH2, and CH3.

The term “immunoglobulin light chain” or “light chain of an immunoglobulin” as used herein is intended to refer to one of the light chains of an immunoglobulin. A light chain is typically comprised of a light chain variable (abbreviated herein as VL) region and a light chain constant region (abbreviated herein as CL). The light chain constant region typically is comprised of one domain, CL.

The term “antibody” as used herein is intended to refer to an immunoglobulin molecule, a fragment of an immunoglobulin molecule, or a derivative of either thereof, which has the ability to specifically bind to an antigen under typical physiological and/or tumor-specific conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to be internalized). The binding region (or binding domain which may be used herein, both having the same meaning) which interacts with an antigen, comprises variable regions of both the heavy and light chains of the immunoglobulin molecule. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab' or Fab fragment, a monovalent fragment consisting of the VL, VH, CL and CH1 domains, or a monovalent antibody as described in WO 2007/059782; (ii) F(ab')₂ fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) a dAb fragment (Ward et al., 1989), which consists essentially of a VH domain and is also called domain antibody (Holt et al., 2003); (vi) camelid or nanobodies (Revets et al., 2005) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al. (1988) and Huston et al. (1988). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein. It also should be understood that the term antibody, unless specified otherwise, also includes polyclonal antibodies, monoclonal antibodies (mAbs), antibody-like polypeptides, such as chimeric antibodies and humanized antibodies, as well as ‘antibody fragments’ or ‘fragments thereof’ retaining the ability to specifically bind to the antigen (antigen-binding fragments) provided by any known technique, such as enzymatic cleavage, peptide synthesis, and recombinant techniques, and retaining the ability to be conjugated to a toxin. An antibody as generated can possess any isotype.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell.

The term “full-length antibody” when used herein, refers to an antibody (e.g., a parent or variant antibody) which contains all heavy and light chain constant and variable domains corresponding to those that are normally found in a wild-type antibody of that isotype.

As used herein, “isotype” refers to the immunoglobulin class (for instance IgG1, IgG2, IgG3, IgG4, IgD, IgA, IgE, or IgM) that is encoded by heavy chain constant region genes.

The term “antigen-binding region” or “binding region” as used herein, refers to a region of an antibody which is capable of binding to the antigen. The antigen can be in solution, adhered to or bound to a surface or, e.g., present on a cell, bacterium, or virion. The terms “antigen” and “target” may, unless contradicted by the context, be used interchangeably in the context of the present invention.

The term “epitope” means a protein determinant capable of specific binding to an antibody. Epitopes usually consist of surface groupings of molecules such as amino acids, sugar side chains or a combination thereof and usually have specific three dimensional structural characteristics, as well as specific charge characteristics. Conformational and non-conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the specific antigen binding peptide (in other words, the amino acid residue is within the footprint of the specific antigen binding peptide).

The term “binding” as used herein refers to the binding of an antibody to a predetermined antigen or target, typically with a binding affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BlAcore 3000 instrument using the antigen as the ligand and the protein as the analyte, and binds to the predetermined antigen with an affinity corresponding to a K_{D} that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the protein, so that when the K_{D} of the protein is very low (that is, the protein is highly specific), then the amount with which the affinity for the antigen is lower than the affinity for a non-specific antigen may be at least 10,000 fold. The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻), as used herein, refers to the association equilibrium constant of a particular antibody-antigen interaction and is obtained by dividing the kₐ by the k_{d}.

The term “internalized” or “internalization” as used herein, refers to a biological process in which molecules such as the AXL-ADC are engulfed by the cell membrane and drawn into the interior of the cell. It may also be referred to as “endocytosis”. The internalization of an antibody can, for example, be evaluated according to the assay described in Example 16 of WO 2016/005593 A1.

The terms “antibody binding AXL”, “AXL-antibody” or “anti-AXL antibody” as used herein, refers to any antibody binding an epitope on the extracellular part of AXL.

In the context of the present invention the term “ADC” refers to an antibody drug conjugate, which in the context of the present invention refers to an anti-AXL antibody which is coupled to a therapeutic moiety, e.g., a cytotoxic moiety as described in the present application. It may e.g. be coupled with a linker to e.g. cysteine or with other conjugation methods to other amino acids. The moiety may e.g. be a drug or a toxin or the like.

As used herein, a “therapeutic moiety” means a compound which exerts a therapeutic or preventive effect when administered to a subject, particularly when delivered as an ADC as described herein. A “cytotoxic” or “cytostatic” moiety is a compound that is detrimental to (e.g., kills) cells. Some cytotoxic or cytostatic moieties for use in ADCs are hydrophobic, meaning that they have no or only a limited solubility in water, e.g., 1 g/L or less (very slightly soluble), such as 0.8 g/L or less, such as 0.6 g/L or less, such as 0.4 g/L or less, such as 0.3 g/L or less, such as 0.2 g/L or less, such as 0.1 g/L or less (practically insoluble). Exemplary hydrophobic cytotoxic or cytostatic moieties include, but are not limited to, certain microtubulin inhibitors such as auristatin and its derivatives, e.g., MMAF and MMAE.

The abbreviation “MMAE” refers to monomethyl auristatin E.

The abbreviations “vc” and “val-cit” refer to the dipeptide valine-citrulline.

The abbreviation “PAB” refers to the self-immolative spacer:

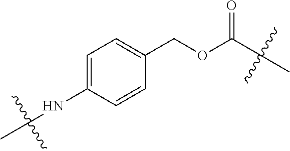 [SMILES: *Nc1ccc(COC(*)=O)cc1]

The abbreviation “MC” refers to the stretcher maleimidocaproyl:

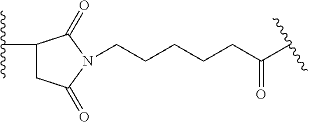 [SMILES: *C(=O)CCCCCN1C(=O)CC(*)C1=O]

“Anti-AXL-MC-vc-PAB-MMAE” refers to an anti-AXL antibody conjugated to the drug MMAE through a MC-vc-PAB linker.

A “tyrosine-kinase inhibitor” or “TKI” as used herein refers to a compound, typically a pharmaceutical, which inhibits tyrosine kinases or down-stream signaling from tyrosine kinases. Tyrosine kinases are enzymes responsible for the addition of a phosphate group to a tyrosine of a protein (phosphorylation), a step that TKIs inhibit, either directly or indirectly. Tyrosine phosphorylation results in the activation of intracellular signal transduction cascades. Many TKIs are useful for cancer therapy. In one embodiment, the term tyrosine kinase inhibitor as used herein refer to compounds which specifically inhibit the protein phosphorylation activity of a tyrosine kinase, e.g., the tyrosine kinase activity of the EGFR, i.e., an EGFR inhibitor. Examples of TKIs useful for cancer therapy include erlotinib and analogs and derivatives thereof.

While many TKIs in clinical use are small molecule pharmaceuticals, there are also “receptor tyrosine kinase inhibitors” (rTKIs) such as antagonistic antibodies which bind to the extracellular portion of a receptor tyrosine kinase (herein referred to as “mAb/rTKIs”), thereby inhibiting receptor-mediated signaling. Examples of such antibodies are cetuximab and MAB391.

A “serine/threonine kinase inhibitor” or “S/Th KI”, as used herein, refers to a compound, typically a pharmaceutical, which inhibits serine/threonine kinases such as BRAF or MEK or down-stream signaling pathways from such serine/threonine kinases such as via the BRAF/MEK pathways. Serine/threonine kinases are enzymes responsible for the phosphorylation of the hydroxyl-group of a serine or threonine residue, a step that S/Th KIs inhibit, either directly or indirectly. Phosphorylation of serines or threonines results in the activation of intracellular signal transduction cascades. Examples of S/Th KIs useful for cancer therapy include BRAF-inhibitors such as vemurafenib and analogs or derivatives thereof. In one embodiment, the term serine/threonine kinase inhibitor as used herein refer to compounds which specifically inhibit the protein phosphorylation activity of a serine/threonine kinase, e.g., the serine/threonine kinase activity of a mutant BRAF or MEK.

As used herein, a “derivative” of a drug is a compound that is derived or derivable, by a direct chemical reaction, from the drug. As used herein, an “analog” or “structural analog” of a drug is a compound having a similar structure and/or mechanism of action to the drug but differing in at least one structural element. “Therapeutically active” analogs or derivatives of a drug such as, e.g., vemurafenib or erlotinib, have a similar or improved therapeutic efficacy as compared to the drug but may differ in, e.g., one or more of stability, target specificity, solubility, toxicity, and the like.

“Treatment” refers to the administration of an effective amount of a therapeutically active compound as described herein to a subject with the purpose of easing, ameliorating, arresting or eradicating (curing) symptoms or disease states of the subject.

As used herein, “maintenance therapy” means therapy for the purpose of avoiding or delaying the cancer's progression or return. Typically, if a cancer is in complete remission after the initial treatment, maintenance therapy can be used to avoid or delay return of the cancer. If the cancer is advanced and/or complete remission has not been achieved after the initial treatment, maintenance therapy can be used to slow the growth of the cancer, e.g., to lengthen the life of the patient.

As used herein, the term “subject” is typically a human to whom an AXL-ADC is administered, and who may benefit from the administration of AXL-ADC, including for instance human patients diagnosed as having a cancer that may be treated by killing of AXL-expressing cells, directly or indirectly.

An “effective amount” or “therapeutically effective amount” refers to an amount effective, at dosages and for periods of time necessary, to achieve a desired therapeutic result. A therapeutically effective amount of an AXL-ADC may vary according to factors such as the disease state, age, sex, and weight of the individual, and the ability of the AXL-ADC to elicit a desired response in the individual. A therapeutically effective amount is also one in which any toxic or detrimental effects of the AXL-ADC are outweighed by the therapeutically beneficial effects.

As used herein, a “resistant”, “treatment-resistant” or “refractory” cancer, tumor or the like, means a cancer or tumor in a subject, wherein the cancer or tumor did not respond to treatment with a therapeutic agent from the onset of the treatment (herein referred to as “native resistance”) or initially responded to treatment with the therapeutic agent but became non-responsive or less responsive to the therapeutic agent after a certain period of treatment (herein referred to as “acquired resistance”), resulting in progressive disease. For solid tumors, also an initial stabilization of disease represents an initial response. Other indicators of resistance include recurrence of a cancer, increase of tumor burden, newly identified metastases or the like, despite treatment with the therapeutic agent. Whether a tumor or cancer is, or has a high tendency of becoming, resistant to a therapeutic agent can be determined by a person of skill in the art. For example, the National Comprehensive Cancer Network (NCCN, www.nccn.org) and European Society for Medical Oncology (ESMO, www.esmo.org/Guidelines) provide guidelines for assessing whether a specific cancer responds to treatment.

As used herein, a cancer which “has a high tendency” for resistance to a specific therapeutic agent is a cancer which is known to be associated with a high tendency of being or becoming resistant or refractory to treatment with a certain class of drugs. For example, a cancer patient who is being treated or who has been found to be eligible for treatment with a therapeutic agent as described herein for which there is a correlation between resistance and enhanced or de novo expression of AXL, suffers from a cancer having a high tendency for resistance. Cancers and classes of therapeutic agents known to be associated with enhanced or de novo expression of AXL and which thus may have a high tendency to become resistant to the therapeutic agent, are known in the art.

The present invention also provides, in one embodiment, antibodies comprising functional variants of the V_{L} region, V_{H} region, or one or more CDRs of the antibodies described herein. A functional variant of a V_{L}, V_{H}, or CDR used in the context of an anti-AXL antibody still allows the antibody to retain at least a substantial proportion (at least about 50%, 60%, 70%, 80%, 90%, 95% or more) of the affinity/avidity and/or the specificity/selectivity of the parent antibody and in some cases such an anti-AXL antibody may be associated with greater affinity, selectivity and/or specificity than the parent antibody.

Such functional variants typically retain significant sequence identity to the parent antibody. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences may be accomplished using a mathematical algorithm, as described in the non-limiting examples below.

The percent identity between two nucleotide sequences may be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences may also be determined using the algorithm of E. Meyers and W. Miller, Comput. Appl. Biosci 4, 11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The sequence of CDR variants may differ from the sequence of the CDR of the parent antibody sequences through mostly conservative amino acid substitutions; for instance at least about 35%, about 50% or more, about 60% or more, about 70% or more, about 75% or more, about 80% or more, about 85% or more, about 90% or more, about 95% or more (e.g., about 65-99%, such as about 96%, 97% or 98%) of the substitutions in the variant are conservative amino acid residue replacements.

The sequence of CDR variants may differ from the sequence of the CDR of the parent antibody sequences through mostly conservative substitutions; for instance at least 10, such as at least 9, 8, 7, 6, 5, 4, 3, 2 or 1 of the substitutions in the variant are conservative amino acid residue replacements. In one embodiment the substitutions are only conservative amino acid residue substitutions. In one embodiment there are a total of no more than 3 conservative amino acid substitutions in a specific CDR region. In one embodiment, the variant has at most 1, 2 or 3 amino acid modifications, such as amino acid-substitutions, optionally conservative amino acid substitutions, in total across the six VH and VL CDR regions of a particular anti-AXL antibody.

The term “amino acid substitution” embraces a substitution into any one or the other nineteen natural amino acids, or into other amino acids, such as non-natural amino acids. For example, an amino acid may be substituted for another conservative or non-conservative amino acid. Amino acid residues may also be divided into classes defined by alternative physical and functional properties. Thus, classes of amino acids may be reflected in one or both of the following lists:

Amino acid residue of conservative class:

Acidic Residues: D and E

Basic Residues: K, R, and H

Hydrophilic Uncharged Residues: S, T, N, and Q

Aliphatic Uncharged Residues: G, A, V, L, and I

Non-polar Uncharged Residues: C, M, and P

Aromatic Residues: F, Y, and W

Alternative Physical and Functional Classifications of Amino Acid Residues:

Alcohol group-containing residues: S and T

Aliphatic residues: I, L, V, and M

Cycloalkenyl-associated residues: F, H, W, and Y

Hydrophobic residues: A, C, F, G, H, I, L, M, R, T, V, W, and Y

Negatively charged residues: D and E

Polar residues: C, D, E, H, K, N, Q, R, S, and T

Positively charged residues: H, K, and R

Small residues: A, C, D, G, N, P, S, T, and V

Very small residues: A, G, and S

Residues involved in turn formation: A, C, D, E, G, H, K, N, Q, R, S, P, and T

Flexible residues: Q, T, K, S, G, P, D, E, and R

### SPECIFIC EMBODIMENTS OF THE INVENTION

In one aspect, the present invention relates to an ADC comprising an antibody binding to human AXL for use in a method of treating a cancer, the method comprising administering the ADC to a subject in need thereof in at least one cycle comprising administration once a week for three consecutive weeks followed by a one week resting period without any administration of ADC so that each cycle time is 28 days including the resting period, wherein the antibody is conjugated to an auristatin or a functional peptide analog or derivate thereof via a linker. The dosage regimen of the invention can alternatively be described as at least one 28-day cycle in which an AXL-ADC is administered to a subject once a week for three consecutive weeks followed by a one week resting period. As used herein, the term “resting period” is to be understood as a period of time wherein the AXL-ADC is administered at a substantially lower dose than that administered the preceding week, or wherein the AXL-ADC is not administered at all, e.g., during which the ADC is not administered at all. In a preferred embodiment of any aspect or embodiment herein, no AXL-ADC is administered during the resting period, in which case the resting period may alternatively be referred to as an “off-period”. A resting period or off-period of one week can also be referred to as a “resting week” or “off-week”, respectively.

Preferred anti-AXL antibodies are characterized by AXL-binding properties, variable or hypervariable sequences, or a combination of binding and sequence properties, set out in the aspects and embodiments below. In a particular embodiment, the antibody binds to AXL but does not compete for AXL binding with the ligand Growth Arrest-Specific 6 (Gash). Most preferred are the specific anti-AXL antibodies comprising VH region and VL region CDRs, VH and/or VL sequences described in Table 2. Of particular interest are antibodies sharing one or more AXL-binding properties or CDR, VH and/or VL sequences with an antibody selected from the group consisting of antibody 107, 148 and 733, or a variant of any thereof.

So, in one embodiment, the anti-AXL antibody comprises a VH region and a VL region selected from the group consisting of
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733];
(d) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
(e) a VH region comprising the CDR1, CDR2, and CDR3 sequences of SEQ ID Nos.: 51, 52, and 54, respectively; and a VL region comprising the CDR1, CDR2, and CDR3 sequences of SEQ ID Nos.: 55, GAS, and 56, respectively [154-M103L];
(f) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171];
(g) a VH region comprising the CDR1, CDR2, and CDR3 sequences of SEQ ID Nos.: 62, 63, and 64, respectively; and a VL region comprising the CDR1, CDR2, and CDR3 sequences of SEQ ID Nos.: 65, GAS, and 66, respectively, [172];
(h) a VH region comprising the CDR1, CDR2, and CDR3 sequences of SEQ ID Nos.: 67, 68, and 69, respectively; and a VL region comprising the CDR1, CDR2, and CDR3 sequences of SEQ ID Nos.: 70, GAS, and 71, respectively, [181];
(i) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183];
(j) a VH region comprising the CDR1, CDR2, and CDR3 sequences of SEQ ID Nos.: 72, 74, and 75, respectively; and a VL region comprising the CDR1, CDR2, and CDR3 sequences of SEQ ID Nos.: 76, ATS, and 77, respectively, [183-N52Q];
(k) a VH region comprising the CDR1, CDR2, and CDR3 sequences of SEQ ID Nos.: 78, 79, and 80, respectively; and a VL region comprising the CDR1, CDR2, and CDR3 sequences of SEQ ID Nos.: 81, AAS, and 82, respectively, [187];
(l) a VH region comprising the CDR1, CDR2, and CDR3 sequences of SEQ ID Nos.: 83, 84, and 85, respectively; and a VL region comprising the CDR1, CDR2, and CDR3 sequences of SEQ ID Nos.: 86, GAS, and 87, respectively, [608-01];
(m) a VH region comprising the CDR1, CDR2, and CDR3 sequences of SEQ ID Nos.: 88, 89, and 90, respectively; and a VL region comprising the CDR1, CDR2, and CDR3 sequences of SEQ ID Nos.: 91, GAS, and 92, respectively, [610-01];
(n) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
(o) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 102, respectively, [613-08];
(p) a VH region comprising the CDR1, CDR2, and CDR3 sequences of SEQ ID Nos.: 103, 104, and 105, respectively; and a VL region comprising the CDR1, CDR2, and CDR3 sequences of SEQ ID Nos.: 106, GAS, and 107, respectively, [620-06];
(q) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726];
(r) a VH region comprising the CDR1, CDR2, and CDR3 sequences of SEQ ID Nos.: 108, 109, and 111, respectively; and a VL region comprising the CDR1, CDR2, and CDR3 sequences of SEQ ID Nos.: 112, AAS, and 113, respectively, [726-M101L];
(s) a VH region comprising the CDR1, CDR2, and CDR3 sequences of SEQ ID Nos.: 41, 42, and 43, respectively; and a VL region comprising the CDR1, CDR2, and CDR3 sequences of SEQ ID Nos.: 44, AAS, and 45, respectively, [140];
(t) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08];
(u) a VH region comprising the CDR1, CDR2, and CDR3 sequences of SEQ ID Nos.: 46, 119, and 120, respectively; and a VL region comprising CDR1, CDR2, and CDR3 sequences of SEQ ID Nos.: 49, AAS, and 50, respectively, [148/140];
(v) a VH region comprising the CDR1, CDR2, and CDR3 sequences of SEQ ID Nos.: 123, 124, and 125, respectively; and a VL region comprising CDR1, CDR2, and CDR3 sequences of SEQ ID Nos.: 60, GAS, and 61, respectively [171/172/181];
(w) a VH region comprising the CDR1, CDR2, and CDR3 sequences of SEQ ID Nos.: 121, 109, and 122, respectively; and a VL region comprising the CDR1, CDR2, and CDR3 sequences of SEQ ID Nos.: 112, AAS, and 113, respectively [726/187];
(x) a VH region comprising the CDR1, CDR2, and CDR3 sequences of SEQ ID Nos.:93, 126, and 127, respectively; and a VL region comprising the CDR1, CDR2, and CDR3 sequences of SEQ ID Nos.: 96, GAS, and 97, respectively [613/608-01/610-01/620-06]; and
(y) a variant of any of said antibodies defined in (a) to (x), wherein said variant preferably has at most 1, 2 or 3 amino-acid modifications, more preferably amino-acid substitutions, such as conservative amino-acid substitutions, across the six CDR sequences.

In one embodiment, the anti-AXL antibody comprises a VH region and a VL region selected from the group consisting of
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148]; and
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733]; and
(d) a variant of any of said antibodies defined in (a) to (c), wherein said variant preferably has at most 1, 2 or 3 amino-acid modifications, more preferably amino-acid substitutions, such as conservative amino-acid substitutions across the six CDR sequences.

In certain embodiments of the invention, the anti-AXL antibody comprises a VH region and a VL region selected from the group consisting of:
(a) a VH region comprising SEQ ID No: 1 and a VL region comprising SEQ ID No: 2 [107];
(b) a VH region comprising SEQ ID No: 5 and a VL region comprising SEQ ID No: 6 [148];
(c) a VH region comprising SEQ ID No: 34 and a VL region comprising SEQ ID No: 35 [733].

In one preferred embodiment of the invention, the anti-AXL antibody of the ADC is the antibody having the VH CDR1, CDR2 and CDR3 amino acid sequences set forth in SEQ ID Nos.: 36, 37, and 38, respectively; and the VL CDR1, CDR2 and CDR3 amino acid sequence of SEQ ID Nos.: 39, GAS, and 40, respectively, [107]. For example, the anti-AXL antibody may comprise a VH region comprising SEQ ID No: 1 and a VL region comprising SEQ ID No: 2 [107].

In one preferred embodiment of the invention, the anti-AXL antibody of the ADC is the antibody having the VH CDR1, CDR2 and CDR3 amino acid sequences set forth in SEQ ID Nos.: 46, 47, and 48, respectively; and the VL CDR1, CDR2 and CDR3 amino acid sequence of SEQ ID Nos.: 49, AAS, and 50, respectively, [148]. For example, the anti-AXL antibody may comprise a VH region comprising SEQ ID No: 5 and a VL region comprising SEQ ID No: 6 [148].

In one preferred embodiment of the invention, the anti-AXL antibody of the ADC is the antibody having the VH CDR1, CDR2 and CDR3 amino acid sequences set forth in SEQ ID Nos.: 114, 115, and 116, respectively; and the VL CDR1, CDR2 and CDR3 amino acid sequence of SEQ ID Nos.: 117, DAS, and 118, respectively, [733]. For example, the anti-AXL antibody may comprise a VH region comprising SEQ ID No: 34 and a VL region comprising SEQ ID No: 35 [733].

In a preferred embodiment of the invention, the antibody is a full length antibody. The antibody may, for example, be a fully human monoclonal IgG1 antibody, such as an IgG1,K. In one embodiment, the antibody is a full-length antibody.

In the AXL-ADCs for use according to the invention, the antibody is conjugated to an auristatin or a functional peptide analog or derivate thereof (see, e.g., U.S. Pat. Nos. 5,635,483 and 5,780,588). Auristatins have been shown to interfere with microtubule dynamics, GTP hydrolysis and nuclear and cellular division (Woyke et al., 2001) and have anti-cancer (U.S. Pat. No. 5,663,149) and anti-fungal activity (Pettit et al., 1998). In one embodiment, the auristatin drug moiety is attached to the antibody via a linker, e.g., through the N (amino) terminus and/or the C (terminus) of the peptidic drug moiety. In one, embodiment the linker of the antibody drug conjugate for use of the invention is attached to sulphydryl residues of the anti-AXL antibody obtained by (partial) reduction of the anti-AXL antibody.

In one embodiment of the invention, the drug moiety comprised in the AXL-ADC is monomethyl auristatin E (MMAE):

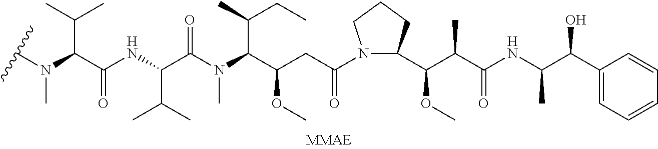 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In an embodiment of the invention the linker-auristatin of the antibody drug conjugate for use of the present invention is vcMMAE:

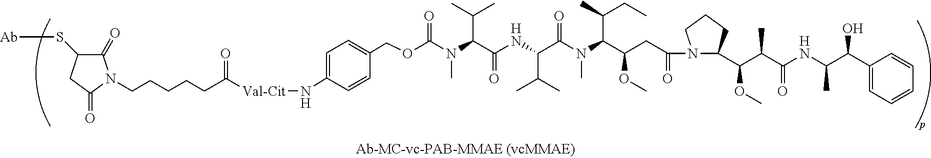

wherein p denotes a number of from 1 to 8, S represents a sulphydryl residue of the anti-AXL antibody, and Ab designates the anti-AXL antibody. In a particular embodiment, p is 4 so that the anti-AXL antibody molecule is conjugated to four molecules of MMAE. Also contemplated are compositions, such as pharmaceutical compositions, comprising a multitude of AXL-ADC molecules wherein, on average, each anti-AXL antibody is conjugated to about four molecules of MMAE via a vc linker, i.e., p is about 4 for the AXL-ADC composition.

In a preferred embodiment of the invention, the anti-AXL antibody is the 107 monoclonal antibody so that the anti-AXL ADC is IgG1-1021-107-MMAE (HuMax-AXL-ADC).

In another preferred embodiment of the invention, the anti-AXL antibody is the 148 monoclonal antibody so that the anti-AXL ADC is IgG1-1021-148-MMAE.

In another preferred embodiment of the invention, the anti-AXL antibody is the 733 monoclonal antibody so that the anti-AXL ADC is IgG1-1021-733-MMAE.

Methods for preparing anti-AXL-ADCs suitable for use according to the invention are described in WO 2016/005593 A1 (Genmab) and WO 2017/009258 (Genmab), each of which is hereby incorporated by reference in its entirety.

Protocol

In a preferred embodiment of the present invention, the AXL-ADC for use of the present invention is administered as single weekly doses for three consecutive weeks in a cycle of 28 days. In some embodiments, the dose will be administered as a single weekly dose on days 1, 8, and 15 of a 28 day treatment cycle.

Hereby, a dosing regimen is provided where the subject to be treated is dosed with a single weekly dose for three consecutive weeks followed by a resting week. This treatment schedule may also be referred to as a “weekly treatment cycle” herein and is the same as “the four-week (28 days) treatment cycle” and “3Q4W”. The present invention encompasses embodiments wherein the subject remains on the 3Q4W treatment cycle for at least 2, 3, 4, 5, 6, 7, 8, 9, 10, 11, 12 or more cycles. In another embodiment, the subject remains on the 3Q4W treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles wherein each cycle is 28 days as described above. In some embodiments, the subject remains on the 3Q4W treatment cycle for 12 cycles or more, such as 16 cycles or more, such as 24 cycles or more, such as 36 cycles or more. In some embodiments, the 3Q4W treatment cycle is administered for no more than 3, no more than 4, no more than 5, or no more than 6 four-week treatment cycles. The number of treatment cycles suitable for any specific subject or group of subjects may be determined by a person of skill in the art, typically a physician. For example, such a person may, for example, evaluate the response to the AXL/ADC treatment based on the criteria provided in Table 1 (RECIST Criteria v1.1).

In certain embodiments of the invention, the weekly dose of the AXL-ADC for use of the invention is between 0.45 mg/kg and 2.0 mg/kg of the subject's body weight such at a dose of 0.45 mg/kg or at a dose of 0.5 mg/kg or at a dose of 0.6 mg/kg or at a dose of 0.7 mg/kg or at a dose of 0.8 mg/kg or at a dose of 0.9 mg/kg or at a dose of 1.0 mg/kg or at a dose of 1.1 mg/kg or at a dose of 1.2 mg/kg or at a dose of 1.3 mg/kg or at a dose of 1.4 mg/kg or at a dose of 1.5 mg/kg or at a dose of 1.6 mg/kg or at a dose of 1.7 mg/kg or at a dose of 1.8 mg/kg or at a dose of 1.9 mg/kg or at a dose of 2.0 mg/kg.

In some embodiments, the weekly dose of the antibody drug conjugate will be about 0.45 mg/kg body weight. In some embodiments, the weekly dose of the antibody drug conjugate will be about 0.5 mg/kg body weight. In some embodiments, the weekly dose of the antibody drug conjugate will be about 0.6 mg/kg body weight. In some embodiments, the weekly dose of the antibody drug conjugate will be about 0.7 mg/kg body weight. In some embodiments, the weekly dose of the antibody drug conjugate will be about 0.8 mg/kg body weight. In some embodiments, the weekly dose of the antibody drug conjugate will be about 0.9 mg/kg body weight. In some embodiments, the weekly dose of the antibody drug conjugate will be about 1.0 mg/kg body weight. In some embodiments, the weekly dose of the antibody drug conjugate will be about 1.1 mg/kg body weight. In some embodiments, the weekly dose of the antibody drug conjugate will be about 1.2 mg/kg body weight. In some embodiments, the weekly dose of the antibody drug conjugate will be about 1.3 mg/kg body weight. In some embodiments, the weekly dose of the antibody drug conjugate will be about 1.4 mg/kg body weight. In some embodiments, the weekly dose of the antibody drug conjugate will be about 1.5 mg/kg body weight. In some embodiments, the weekly dose of the antibody drug conjugate will be about 1.6 mg/kg body weight. In some embodiments, the weekly dose of the antibody drug conjugate will be about 1.7 mg/kg body weight. In some embodiments, the weekly dose of the antibody drug conjugate will be about 1.8 mg/kg body weight. In some embodiments, the weekly dose of the antibody drug conjugate will be about 1.9 mg/kg body weight. In some embodiments, the weekly dose of the antibody drug conjugate will be about 2.0 mg/kg body weight.

In one embodiment of the invention the antibody drug conjugate is administered at a dose of about 0.45 mg/kg body weight for at least four treatment cycles of 28 days in which cycles the antibody drug conjugate is administered once a week as a single dose for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.45 mg/kg body weight for at least 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.6 mg/kg body weight for at least 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 0.8 mg/kg body weight for at least 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least eight treatment cycles of 28 days in which cycles the antibody drug conjugate is administered once a week for three consecutive weeks followed by a resting week.

In one embodiment of the invention the antibody drug conjugate is administered at a dose of about 1.0 mg/kg body weight for at least nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.0 mg/kg body weight for at least 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for four treatment cycles of 28 days in which cycles the antibody drug conjugate is administered once a week as a single dose for three consecutive weeks followed by a resting week.

In one embodiment of the invention the antibody drug conjugate is administered at a dose of about 1.2 mg/kg body weight for five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.2 mg/kg body weight for 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.4 mg/kg body weight for 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.6 mg/kg body weight for 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 1.8 mg/kg body weight for 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for four treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for five treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for six treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for seven treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for eight treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for nine treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for 10 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for 11 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In one embodiment of the invention the antibody drug conjugate is administered at a dose of about 2.0 mg/kg body weight for 12 treatment cycles of 28 days in which cycles the antibody drug conjugate is administered once a week for three consecutive weeks followed by a resting week. For example, the dose may be administered as a single weekly dose on days 1, 8, and 15 of a 28 day treatment cycle.

In some embodiments of the invention, the antibody drug conjugate is administered at a dose of about 0.6 mg/kg, about 0.8 mg/kg, about 1.0 mg/kg, about 1.2 mg/kg, about 1.4 mg/kg, about 1.6 mg/kg, about 1.8 mg/kg or about 2.0 mg/kg body weight for at least 12 treatment cycles of 28 days, such as at least 24 treatment cycles of 28 days, such as at least 36 cycles of 28 days, such as at least 36 treatment cycles, such as up to 48 treatment cycles, in which cycles the antibody drug conjugate is administered once a week for three consecutive weeks followed by a resting week. For example, the dose may be administered as a single weekly dose on days 1, 8, and 15 of a 28 day treatment cycle.

In certain embodiments of the invention, the total amount administered to a subject in those weeks where AXL-ADC is administered (i.e., the weekly dose) of the AXL-ADC for use according to the present invention is a fixed dose of between 50 mg and 200 mg, such at a dose of 60 mg or a dose of 70 mg or a dose of 80 mg or a dose of 90 mg or a dose of 100 mg or a dose of 110 mg or a dose of 120 mg or a dose of 130 mg or a dose of 140 mg or a dose of 150 mg or a dose of 160 mg or a dose of 170 mg or a dose of 180 mg or a dose of 190 mg or a dose of 200 mg.

Maintenance Therapy

A person of skill in the art, such as a physician, may determine that, after a suitable number of treatment cycles, the treatment cycles should be followed by maintenance therapy with AXL-ADC, treatment with another therapeutic agent or combination of therapeutic agents, as appropriate.

In some embodiments, the subject will begin maintenance therapy following one or more, preferably two or more, such as following 3 or 4 or 5 or 6 or 7 or 8 or 9 or 10 or 11 or 12 or more cycles, such as 24 cycles or more, such as 36 cycles or more, of four-week treatment cycles (3Q4W).

In some embodiments, the subject will start maintenance therapy following an evaluation indicating that the subject has little or no detectable cancer, e.g., following an evaluation indicating that the subject has had a complete response.

As used herein, “maintenance therapy” refers to therapy with the AXL-ADC but at a reduced administration schedule at either the same or different dosages. During maintenance therapy, the AXL-ADC is preferably administered once every two weeks, once every three weeks, once every four weeks, once every five weeks, once every 6 weeks, once every 7 weeks, or once every 8 weeks. In one embodiment, during maintenance therapy, the AXL-ADC is administered once every 3 weeks (which may be referred to as “1Q3W”), such as on day 1 of a 21-day cycle. In one embodiment, during maintenance therapy, the AXL-ADC is administered once every 4 weeks (which may be referred to as “1Q4W”), such as on day 1 of a 28-day cycle.

The dose of the AXL-ADC for the maintenance therapy may, for example, be in the range of about 0.6 mg/kg body weight to about 3.2 mg/kg body weight. In some embodiments, the dose of the AXL-ADC for the maintenance therapy is from about 0.8 mg/kg to about 3.2 mg/kg 1 mg/kg body weight to about 3.2 mg/kg body weight, such as from about 1.2 mg/kg to about 3.0 mg/kg, such as from about 1.4 mg/kg to about 2.8 mg/kg, such as from about 1.6 to about 2.6 mg/kg, such as from about 1.8 mg/kg to about 2.4 mg/kg, such as from about 2.0 mg/kg to about 2.2 mg/kg, or from about 1.0 mg/kg to about 2.0 mg/kg, such as from about 1.1 mg/kg to about 2.1 mg/kg, such as from about 1.2 mg/kg to about 2.2 mg/kg, such as from about 1.3 mg/kg to about 2.3 mg/kg, such as from about 1.4 mg/kg to about 2.4 mg/kg, such as from about 1.5 mg/kg to about 2.5 mg/kg, such as from about 1.6 mg/kg to about 2.6 mg/kg, such as from about 1.7 mg/kg to about 2.7 mg/kg, such as from about 1.8 mg/kg to about 2.8 mg/kg, such as from about 1.9 mg/kg to about 2.9 mg/kg, such as from about 2.0 mg/kg to about 3.0 mg/kg body weight. In a specific embodiment the dose of the AXL-ADC for the maintenance therapy is about 1.1 mg/kg body weight. In a specific embodiment the dose of the AXL-ADC for the maintenance therapy is about 1.2 mg/kg body weight. In a specific embodiment the dose of the AXL-ADC for the maintenance therapy is about 1.3 mg/kg body weight. In a specific embodiment the dose of the AXL-ADC for the maintenance therapy is about 1.4 mg/kg body weight. In a specific embodiment the dose of the AXL-ADC for the maintenance therapy is about 1.5 mg/kg body weight. In a specific embodiment the dose of the AXL-ADC for the maintenance therapy is about 1.6 mg/kg body weight. In a specific embodiment the dose of the AXL-ADC for the maintenance therapy is about 1.7 mg/kg. In a specific embodiment the dose of the AXL-ADC for the maintenance therapy is about 1.8 mg/kg body weight. In a specific embodiment the dose of the AXL-ADC for the maintenance therapy is about 1.9 mg/kg body weight. In a specific embodiment the dose of the AXL-ADC for the maintenance therapy is about 2.0 mg/kg body weight. In a specific embodiment the dose of the AXL-ADC for the maintenance therapy is about 2.1 mg/kg body weight. In a specific embodiment the dose of the AXL-ADC for the maintenance therapy is about 2.2 mg/kg body weight. In a specific embodiment the dose of the AXL-ADC for the maintenance therapy is about 2.3 mg/kg body weight. In a specific embodiment the dose of the AXL-ADC for the maintenance therapy is about 2.4 mg/kg body weight. In a specific embodiment the dose of the AXL-ADC for the maintenance therapy is about 2.5 mg/kg body weight. In a specific embodiment the dose of the AXL-ADC for the maintenance therapy is about 2.6 mg/kg body weight. In a specific embodiment the dose of the AXL-ADC for the maintenance therapy is about 2.7 mg/kg body weight. In a specific embodiment the dose of the AXL-ADC for the maintenance therapy is about 2.8 mg/kg body weight. In a specific embodiment the dose of the AXL-ADC for the maintenance therapy is about 2.9 mg/kg body weight. In a specific embodiment the dose of the AXL-ADC for the maintenance therapy is about 3.0 mg/kg body weight. In a specific embodiment the dose of the AXL-ADC for the maintenance therapy is about 3.1 mg/kg body weight. In a specific embodiment the dose of the AXL-ADC for the maintenance therapy is about 3.2 mg/kg body weight.

It is preferred that the AXL-ADC is administered once every three weeks, such as on day 1 of a 21 days cycle. Accordingly, in some embodiments, the weekly dosing cycles of three doses in 28 days can be said to be initial treatment cycles, which are followed by maintenance therapy, comprising administration of the AXL-ADC once every three weeks, i.e., in three weeks cycles or 21 days cycles.

In certain embodiments, the dosage of the AXL-ADC administered during maintenance therapy may range e.g. from about 0.6 mg/kg body weight to about 3.2 mg/kg body weight, such as from about 0.8 mg/kg to about 3.2 mg/kg body weight, in cycles of 21 days as a single dose on day 1 and then again on day 22, and so on.

In some preferred embodiments, the dose of the AXL-ADC for the maintenance therapy is from about 1 mg/kg body weight to about 3.2 mg/kg body weight, such as from about 1.2 mg/kg to about 3.0 mg/kg, such as from about 1.4 mg/kg to about 2.8 mg/kg, such as from about 1.6 mg/kg to about 2.6 mg/kg, such as from about 1.8 mg/kg to about 2.4 mg/kg, such as from about 2.0 mg/kg to about 2.2 mg/kg, or from about 1.0 mg/kg to about 2.0 mg/kg, such as from about 1.1 mg/kg to about 2.1 mg/kg, such as from about 1.2 mg/kg to about 2.2 mg/kg, such as from about 1.3 mg/kg to about 2.3 mg/kg, such as from about 1.4 mg/kg to about 2.4 mg/kg, such as from about 1.5 mg/kg to about 2.5 mg/kg, such as from about 1.6 mg/kg to about 2.6 mg/kg, such as from about 1.7 mg/kg to about 2.7 mg/kg, such as from about 1.8 mg/kg to about 2.8 mg/kg, such as from about 1.9 mg/kg to about 2.9 mg/kg, such as from about 2.0 mg/kg to about 3.0 mg/kg, administered on day 1 of a 21-day cycle. In a specific embodiment the dose of the AXL-ADC for the maintenance therapy is about 1.1 mg/kg. In a specific embodiment the dose of the AXL-ADC for the maintenance therapy is about 1.2 mg/kg. In a specific embodiment the dose of the AXL-ADC for the maintenance therapy is about 1.3 mg/kg. In a specific embodiment the dose of the AXL-ADC for the maintenance therapy is about 1.4 mg/kg. In a specific embodiment the dose of the AXL-ADC for the maintenance therapy is about 1.5 mg/kg. In a specific embodiment the dose of the AXL-ADC for the maintenance therapy is about 1.6 mg/kg. In a specific embodiment the dose of the AXL-ADC for the maintenance therapy is about 1.7 mg/kg. In a specific embodiment the dose of the AXL-ADC for the maintenance therapy is about 1.8 mg/kg. In a specific embodiment the dose of the AXL-ADC for the maintenance therapy is about 1.9 mg/kg. In a specific embodiment the dose of the AXL-ADC for the maintenance therapy is about 2.0 mg/kg. In a specific embodiment the dose of the AXL-ADC for the maintenance therapy is about 2.1 mg/kg. In a specific embodiment the dose of the AXL-ADC for the maintenance therapy is about 2.2 mg/kg. In a specific embodiment the dose of the AXL-ADC for the maintenance therapy is about 2.3 mg/kg. In a specific embodiment the dose of the AXL-ADC for the maintenance therapy is about 2.4 mg/kg. In a specific embodiment the dose of the AXL-ADC for the maintenance therapy is about 2.5 mg/kg. In a specific embodiment the dose of the AXL-ADC for the maintenance therapy is about 2.6 mg/kg. In a specific embodiment the dose of the AXL-ADC for the maintenance therapy is about 2.7 mg/kg. In a specific embodiment the dose of the AXL-ADC for the maintenance therapy is about 2.8 mg/kg. In a specific embodiment the dose of the AXL-ADC for the maintenance therapy is about 2.9 mg/kg. In a specific embodiment the dose of the AXL-ADC for the maintenance therapy is about 3.0 mg/kg. In a specific embodiment the dose of the AXL-ADC for the maintenance therapy is about 3.1 mg/kg. In a specific embodiment the dose of the AXL-ADC for the maintenance therapy is about 3.2 mg/kg.

In certain embodiments, the dosage of the AXL-ADC administered during maintenance therapy may range e.g. from about 50 mg to about 200 mg, such as from about 60 mg to about 190 mg, such as from about 70 mg to about 180 mg, such as from about 80 mg to about 170 mg, such as from about 90 mg to about 160 mg, such as from about 100 to about 150 mg, such as from about 110 mg to about 140 mg, such as from about 120 mg to about 130 mg, or from about 50 mg to about 80 mg, such as from about 60 mg to about 90 mg, such as from about 70 mg to about 100 mg, such as from about 80 mg to about 110 mg, such as from about 90 mg to about 120 mg, such as from about 100 mg to about 130 mg, such as from about 110 mg to about 140 mg, such as from about 120 mg to about 150 mg, such as from about 130 mg to about 160 mg, such as from about 140 mg to about 170 mg, such as from about 150 mg to about 180 mg, such as from about 160 mg to about 190 mg, such as from about 170 mg to about 200 mg.

The AXL-ADC is preferably administered in cycles of 21 days as a single dose on day 1 and then again on day 22 and so on.

So, in some preferred embodiments, the dose of the AXL-ADC for the maintenance therapy is from about 50 mg to about 200 mg, such as from about 60 mg to about 190 mg, such as from about 70 mg to about 180 mg, such as from about 80 mg to about 170 mg, such as from about 90 mg to about 160 mg, such as from about 100 to about 150 mg, such as from about 110 mg to about 140 mg, such as from about 120 mg to about 130 mg, or from about 50 mg to about 80 mg, such as from about 60 mg to about 90 mg, such as from about 70 mg to about 100 mg, such as from about 80 mg to about 110 mg, such as from about 90 mg to about 120 mg, such as from about 100 mg to about 130 mg, such as from about 110 mg to about 140 mg, such as from about 120 mg to about 150 mg, such as from about 130 mg to about 160 mg, such as from about 140 mg to about 170 mg, such as from about 150 mg to about 180 mg, such as from about 160 mg to about 190 mg, such as from about 170 mg to about 200 mg, administered on day 1 of a 21-day cycle. In a specific embodiment the dose of the AXL-ADC for the maintenance therapy is a dose of about 60 mg. In a specific embodiment the dose of the AXL-ADC for the maintenance therapy is a dose of about 70 mg. In a specific embodiment the dose of the AXL-ADC for the maintenance therapy is a dose of about 80 mg. In a specific embodiment the dose of the AXL-ADC for the maintenance therapy is a dose of about 90 mg. In a specific embodiment the dose of the AXL-ADC for the maintenance therapy is a dose of about 100 mg. In a specific embodiment the dose of the AXL-ADC for the maintenance therapy is a dose of about 110 mg. In a specific embodiment the dose of the AXL-ADC for the maintenance therapy is a dose of about 120 mg. In a specific embodiment the dose of the AXL-ADC for the maintenance therapy is a dose of about 130 mg. In a specific embodiment the dose of the AXL-ADC for the maintenance therapy is a dose of about 140 mg. In a specific embodiment the dose of the AXL-ADC for the maintenance therapy is a dose of about 150 mg. In a specific embodiment the dose of the AXL-ADC for the maintenance therapy is a dose of about 160 mg. In a specific embodiment the dose of the AXL-ADC for the maintenance therapy is a dose of about 170 mg. In a specific embodiment the dose of the AXL-ADC for the maintenance therapy is a dose of about 180 mg. In a specific embodiment the dose of the AXL-ADC for the maintenance therapy is a dose of about 190 mg. In a specific embodiment the dose of the AXL-ADC for the maintenance therapy is a dose of about 200 mg.

In certain embodiments the maintenance therapy is administered in cycles of 21 days and the number of cycles are between 2 and 48, such as between 2 and 36, such as between 2 and 24, such as between 2 and 20 such as between 2 and 15 cycles, such as 2 cycles, 3 cycles, 4 cycles, 5 cycles, 6 cycles, 7 cycles, 8 cycles, 9 cycles, 10 cycles, 11 cycles or 12 cycles or 13 cycles or 14 cycles or 15 cycles. In some embodiments, the number of cycles is 12 more, such as 16 or more, such as 24 or more, such as 36 or more. In some embodiments, the maintenance therapy is administered in cycles of 21 days for up to about two years, up to about 3 years, or more.

In certain embodiments the maintenance therapy is administered in cycles of 28 days and the number of cycles are between 2 and 48, such as between 2 and 36, such as between 2 and 24, such as between 2 and 20 such as between 2 and 15 cycles, such as 2 cycles, 3 cycles, 4 cycles, 5 cycles, 6 cycles, 7 cycles, 8 cycles, 9 cycles, 10 cycles, 11 cycles or 12 cycles or 13 cycles or 14 cycles or 15 cycles. In some embodiments, the number of cycles is 12 more, such as 16 or more, such as 24 or more, such as 36 or more. In some embodiments, the maintenance therapy is administered in cycles of 28 days for up to about two years, up to about 3 years, or more.

In another embodiment the maintenance therapy is administered in cycles of 21 days or 28 days, such as of 21 days, until partial or full remission of the cancer is detected or until an evaluation of the subject reveals that further maintenance therapy is unnecessary.

Therapeutic Applications

Accordingly, the present invention include embodiments wherein a subject will be administered a single weekly dose of the AXL-ADC for three consecutive weeks followed by a one week resting period in four week treatment cycles for a number of cycles, optionally followed by maintenance treatment where the subject is dosed with AXL-ADC once every three weeks in three weeks cycles for a number of cycles.

The subject to be treated according to a dosage regimen of the present invention is typically a subject expected to benefit from the administration of AXL-ADC. In separate and specific exemplary embodiments, the subject to be treated according to a dosage regimen of the present invention is selected from
a subject that has been diagnosed with a cancer that expresses AXL,
a subject suspected of having a cancer that expresses AXL, or
a subject diagnosed with a cancer which is resistant, or which has a high tendency to become resistant, to certain therapeutic agent(s).

For example, a cancer that expresses AXL may be a solid tumor expressing AXL or it may be an AXL-expressing hematological cancer.

In some embodiments, the cancer comprises a solid tumor expressing AXL, and is selected from the group consisting of lung cancer, such as non-small cell lung cancer (NSCLC) and lung squamous cell carcinoma; a gynaecological cancer such as ovarian cancer, endometrial cancer or cervical cancer; thyroid cancer; a skin cancer, such as melanoma, e.g., malignant melanoma; colorectal cancer, such as colorectal carcinoma and colorectal adenocarcinoma; bladder cancer; bone cancer such as chondrosarcoma; breast cancer such as triple-negative breast cancer; cancers of the central nervous system such as glioblastoma, astrocytoma and neuroblastoma; connective tissue cancer; fibroblast cancer; gastric cancer such as gastric carcinoma; head and neck cancer; kidney cancer; liver cancer, such as hepatocellular carcinoma; muscle cancer; neural tissue cancer; pancreatic cancer such as pancreatic ductal carcinoma and pancreatic adenocarcinoma; and sarcoma, such as soft tissue sarcoma. In one embodiment, the cancer is melanoma. In one embodiment, the cancer is lung cancer, such as non-small cell lung cancer (NSCLC). In one embodiment, the cancer is sarcoma, such as a sarcoma selected from the group consisting of undifferentiated pleomorphic sarcoma, liposarcoma, leiomyosarcoma, synovial sarcoma, Ewing's sarcoma, osteosarcoma and chondrosarcoma. In one embodiment, the cancer is ovarian cancer. In one embodiment, the cancer is endometrial cancer. In one embodiment, the solid cancer is cervical cancer. In one embodiment, the cancer is thyroid cancer.

In some embodiments, the AXL-expressing hematological cancer is selected from the group consisting of leukemia, such as chronic lymphocytic leukemia (CML), myeloid leukemia, acute myeloid leukemia (AML) and chronic myeloid leukemia, lymphoma such as Non-Hodgkin's lymphoma (NHL) and multiple myeloma.

In certain types of cancer, the development of resistance to therapeutic agents has been associated with increased or de novo expression of AXL. Such cancers include, but are not limited to, cancers characterized by solid tumors, such as NSCLC, ovarian cancer, cervical cancer, melanoma, squamous cell carcinoma of the head and neck (SCCHN), breast cancer, gastrointestinal stromal tumors (GIST), renal cancer, prostate cancer, neuroblastoma, pancreatic cancer, oesophageal cancer, and rhabdomyosarcoma; as well as hematological cancers, such as AML and CML. So, in some embodiments, the subject to be treated according to the dosage regimen of the invention is diagnosed with a cancer that is resistant, or which has a high tendency to become resistant, to certain therapeutic agents.

The development of resistance associated with increased or de novo AXL expression has, for example, been observed for therapeutic agents which are tyrosine kinase inhibitors, PI3K inhibitors, antagonistic antibodies binding to a receptor tyrosine kinase, serine/threonine kinase inhibitors and chemotherapeutic agents.

Accordingly, in some embodiments, the cancer is resistant, or has a high tendency to become resistant, to at least one therapeutic agent selected from the group consisting of a tyrosine kinase inhibitor (TKI), a PI3K inhibitor, an antagonistic antibody binding to a receptor tyrosine kinase, a serine/threonine kinase inhibitor (S/Th KI) and a chemotherapeutic agent. In some embodiments, the cancer is resistant, or has a high tendency to become resistant, to at least one therapeutic agent selected from the group consisting of a tyrosine kinase inhibitor, a serine/threonine kinase inhibitor and a chemotherapeutic agent.

In particular embodiments, the cancer is resistant, or has a high tendency to become resistant, to at least one therapeutic agent selected from the group consisting of an EGFR inhibitor, a BRAF inhibitor, a MEK inhibitor and a chemotherapeutic agent. In one embodiment, the cancer is resistant to at least one therapeutic agent selected from the group consisting of an EGFR inhibitor, a BRAF inhibitor, a MEK-inhibitor and a chemotherapeutic agent.

In one embodiment, the cancer is NSCLC resistant to an EGFR inhibitor. Preferably, the EGFR inhibitor has a similar mechanism of action as erlotinib. Examples of such EGFR inhibitors include erlotinib, gefitinib, afatinib, lapatinib, icotinib, vandetanib, osimertinib and rociletinibare. Preferred but non-limiting examples of EGFR inhibitors are erlotinib, gefitinib and afatinib. In a particular embodiment, the cancer or tumor is characterized by at least one mutation in the EGFR amino acid sequence selected from L858R and T790M, such as e.g., L858R or T790M/L858R.

In one embodiment, the cancer is resistant to a chemotherapeutic agent selected from the group consisting of paclitaxel, docetaxel, cisplatin, doxorubicin, etoposide, carboplatin and metformin. In one embodiment, the therapeutic agent is a microtubule-targeting agent, such as, e.g., paclitaxel, docetaxel or vincristine, or a therapeutically active analog or derivative of any thereof. In one embodiment, the at least one therapeutic agent is a taxane, such as paclitaxel, docetaxel or a therapeutically active analog or derivative of paclitaxel or docetaxel.

In one particular embodiment, the chemotherapeutic agent is paclitaxel, and the cancer is cervical cancer, resistant to or having a high tendency for becoming resistant to paclitaxel.

In one particular embodiment, the chemotherapeutic agent is paclitaxel, and the cancer is an NSCLC, resistant to or having a high tendency for becoming resistant to paclitaxel.

In one embodiment, the cancer is an ovarian cancer resistant to a taxane and/or a platinum derivative, or having a high tendency for becoming resistant to a taxane and/or a platinum derivative. A preferred but non-limiting example of a taxane is paclitaxel. A preferred but non-limiting example of a platinum derivative is cisplatin.

In one particular embodiment, the chemotherapeutic agent is paclitaxel, and the cancer is an ovarian cancer, resistant to or having a high tendency for becoming resistant to paclitaxel.

In one particular embodiment, the chemotherapeutic is docetaxel and the cancer is a head and neck cancer, resistant to or having a high tendency for becoming resistant to docetaxel.

In one particular embodiment, the chemotherapeutic is docetaxel and the cancer is a gastric cancer, resistant to or having a high tendency for becoming resistant to docetaxel.

In one particular embodiment, the chemotherapeutic is docetaxel and the cancer is a breast cancer, resistant to or having a high tendency for becoming resistant to docetaxel.

In one particular embodiment, the chemotherapeutic is docetaxel and the cancer is a prostate cancer, resistant to or having a high tendency for becoming resistant to docetaxel.

In one particular embodiment, the chemotherapeutic is docetaxel and the cancer is a NSCLC, resistant to or having a high tendency for becoming resistant to docetaxel.

In one particular embodiment, the chemotherapeutic agent is cisplatin, and the cancer is an oesophageal cancer, resistant to or having a high tendency for becoming resistant to cisplatin.

In one particular embodiment, the chemotherapeutic agent is cisplatin, and the cancer is an SCCHN, resistant to or having a high tendency for becoming resistant to cisplatin.

In one particular embodiment, the chemotherapeutic agent is carboplatin, and the cancer is an SCCHN, resistant to or having a high tendency for becoming resistant to carboplatin.

In one particular embodiment, the chemotherapeutic agent is cisplatin, and the cancer is an AML, resistant to or having a high tendency for becoming resistant to cisplatin.

In one particular embodiment, the chemotherapeutic agent is doxorubicin, and the cancer is an AML, resistant to or having a high tendency for becoming resistant to doxorubicin.

In one particular embodiment, the chemotherapeutic agent is etoposide, and the cancer is an AML, resistant to or having a high tendency for becoming resistant to etoposide.

In one particular embodiment, the chemotherapeutic agent is metformin, and the cancer is a prostate cancer, resistant to or having a high tendency for becoming resistant to metformin.

In one particular embodiment, the chemotherapeutic agent is cisplatin, and the cancer is an ovarian cancer, resistant to or having a high tendency for becoming resistant to cisplatin.

In one particular embodiment, the chemotherapeutic agent is doxorubicin, and the cancer is a non-small cell lung cancer (NSCLC), resistant to or having a high tendency for becoming resistant to doxorubicin.

In one embodiment, the cancer is cervical cancer resistant to a taxane. Preferred but non-limiting examples of taxanes are paclitaxel and docetaxel.

In one embodiment, the cancer is melanoma resistant to, or having a high tendency for becoming resistant to, at least one of a BRAF-inhibitor and a MEK-inhibitor. For example, the melanoma may be resistant to, or have a high tendency to become resistant to, both a BRAF-inhibitor and a MEK-inhibitor. Preferably, the BRAF inhibitor has a similar mechanism of action as vemurafenib. Examples of such BRAF inhibitors include vemurafenib (PLX4032), GDC-0879 ((E)-5-(1-(2-hydroxyethyl)-3-(pyridin-4-yl)-1H-pyrazol-4-yl)-2,3-dihydroinden-1-one oxime), dabrafenib (GSK2118436), encorafenib (LGX818), sorafenib (BAY 43-9006), RAF265 (CHIR-265), SB590885 ((E)-5-(2-(4-(2-(dimethylamino)ethoxy)phenyl)-4-(pyridin-4-yl)-1H-imidazol-5-yl)-2,3-dihydroinden-1-one oxime) and AZ628 (3-(2-cyanopropan-2-yl)-N-(4-methyl-3-(3-methyl-4-oxo-3,4-dihydroquinazolin-6-ylamino)phenyl)benzamide). Preferred but non-limiting examples of BRAF-inhibitor are vemurafenib and dabrafenib. Examples of MEK-inhibitors include trametinib, cobimetinib, binimetinib and selumetinib. Preferably, the MEK inhibitor has a similar mechanism of action as trametinib. A preferred but non-limiting example of a MEK-inhibitor is trametinib.

Vemurafenib (PLX4032) is an orally bioavailable, ATP-competitive, small-molecule inhibitor of mutated BRAF kinase, which selectively binds to and inhibits BRAF comprising certain mutations, resulting in an inhibition of an over-activated MAPK signaling pathway downstream in the mutant BRAF kinase-expressing tumor cells. BRAF mutations identified in human cancers are generally located in the glycine-rich P loop of the N lobe and the activation segment and flanking regions within the kinase domain. Vemurafenib binds to and inhibits BRAF kinase having certain of these mutations, such as, but not limited to, an amino acid substitution in residue V600 (e.g., V600E, V600K, V600D, and V600R), residue L597 (e.g., L597R; Bahadoran et al., 2013); and residue K601 (e.g., K601E; Dahlman et al., 2012). In one embodiment, the mutation is in V600. In one embodiment, the mutation in BRAF is selected from V600E, V600D, V600K, L597R and K601E.

Accordingly, in some particular embodiments wherein the AXL-ADC is for use in treating a melanoma according to a dosage regimen of the invention, and the melanoma is, or has a tendency of becoming, resistant to vemurafenib or a therapeutically effective analog or derivative thereof, the melanoma may exhibit a mutation in BRAF which renders the BRAF sensitive for inhibition by vemurafenib or the therapeutically effective analog or derivative. Non-limiting mutations include amino acid substitutions, deletions or insertions; preferably, the mutation is an amino acid substitution. Specific residues for such mutations include, but are not limited to, V600 (e.g., V600E, V600K and V600D), residue L597 (e.g., L597R); and residue K601 (K601E). In one embodiment, the mutation is selected from V600E, V600D, V600K, L597R and K601E.

The AXL-ADC can be administered, e.g., as monotherapy. By the term “monotherapy” it is meant that the AXL-ADC is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general illness can also be administered during the period of monotherapy. Also, agents administered to treat potential side-effects of the AXL-ADC can be administered during the period of monotherapy. A subject being treated by the present methods will preferably have completed any prior treatment with anti-cancer agents before administration of the AXL-ADC. In some embodiments, the subject will have completed any prior treatment with anti-cancer agents at least 1 week (preferably 2, 3, 4, 5, 6, 7, or 8 weeks) prior to treatment with the AXL-ADC. The subject will also, preferably, not be treated with any additional anti-cancer agents for at least 2 weeks (preferably at least 3, 4, 5, 6, 7, or 8 weeks) following completion of the first treatment cycle with the antibody drug conjugate and preferably for at least 2 weeks (preferably at least 3, 4, 5, 6, 7, or 8 weeks) following completion of the last dose of the antibody drug conjugate.

The AXL-ADC may alternatively be administered as a combination therapy. By the term “combination therapy” is meant that at least one other anti-cancer agent is administered to the subject during the treatment cycle with AXL-ADC. The AXL-ADC and the at least one other cancer agent may be administered simultaneously, and may optionally be provided in the same pharmaceutical composition. Typically, however, the AXL-ADC and the at least one other anti-cancer agent are separately administered and formulated as separate pharmaceutical compositions. For example, the at least one other anti-cancer agent may be administered according to the dosage regimen for which is has been approved by a medicines regulatory authority when administered as a monotherapy, or the at least one other anti-cancer agent may be administered according to a dosage regimen which is optimized for its combined use with the AXL-ADC.

The pharmaceutical composition(s) comprising the AXL-ADC and the at least one other anti-cancer agent may, for example, be provided in the form of a kit. Accordingly, in one embodiment, the AXL-ADC for use according to the present invention is provided in the form of a kit comprising at least one other anti-cancer agent, wherein the AXL-ADC and at least one other anti-cancer agent are provided for simultaneous, separate or sequential administration, preferably separate or sequential administration. The kit can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

In one aspect, the at least one second anti-cancer agent of the combination, kit or composition is selected from the group consisting of a tyrosine kinase inhibitor, a PI3K inhibitor, an antagonistic antibody binding to a receptor tyrosine kinase, a serine/threonine kinase inhibitor (S/Th KI) and a chemotherapeutic agent.

In a particular aspect, the at least one second anti-cancer agent is a therapeutic agent with which the cancer or tumor has already been treated, and, optionally, has developed resistance to, e.g., a chemotherapeutic agent, tyrosine kinase inhibitor, PI3K inhibitor, mAb/rTKI and/or serine/threonine kinase inhibitor according to any aspect or embodiment herein.

So, for example, in one embodiment, the at least one second anti-cancer agent in the combination, composition or kit comprises a tyrosine kinase inhibitor, such as an EGFR inhibitor having a similar mechanism of action as erlotinib. Examples of such EGFR inhibitors include erlotinib, gefitinib, afatinib, lapatinib, icotinib, vandetanib, osimertinib and rociletinibare. Preferred examples of EGFR inhibitors are erlotinib, gefitinib and afatinib. In one specific embodiment, the cancer or tumor is resistant to one or more tyrosine kinase inhibitors, e.g., the tyrosine kinase inhibitor or EGFR inhibitor provided in the combination, composition or kit. In another specific embodiment, the cancer or tumor is NSCLC, optionally resistant to the tyrosine kinase inhibitor or EGFR inhibitor provided in the combination, composition or kit. In a particular embodiment, the cancer or tumor is characterized by at least one mutation in the EGFR amino acid sequence selected from L858R and T790M, such as e.g., L858R or T790M/L858R.

In one embodiment, the at least one second anti-cancer agent in the combination, kit or composition comprises one or more serine/threonine kinase inhibitors, such as a BRAF inhibitor, a MEK-inhibitor or a combination of a BRAF inhibitor and a MEK-inhibitor.

In one embodiment, the BRAF inhibitor inhibits the serine/threonine kinase activity of one or more mutants of human BRAF, such as those having a mutation in residue V600, L597 or K601, such as V600E. For example, a BRAFi may inhibit the serine/threonine kinase activity of the mutant BRAFi more effectively than they inhibit native human BRAF, thus being selective for the mutant BRAF. The BRAF inhibitor may also or alternatively inhibit the serine/threonine kinase activity of one or both of A-RAF (UniProtKB P10398 (ARAF_HUMAN)) and C-RAF (UniProtKB P04049 (RAF1_HUMAN)) and/or mutants thereof. Preferably, the BRAF inhibitor has a similar mechanism of action as vemurafenib. Examples of such BRAF inhibitors include vemurafenib, GDC-0879 ((E)-5-(1-(2-hydroxyethyl)-3-(pyridin-4-yl)-1H-pyrazol-4-yl)-2,3-dihydroinden-1-one oxime), dabrafenib, encorafenib, sorafenib, RAF265 (CHIR-265), SB590885 ((E)-5-(2-(4-(2-(dimethylamino)ethoxy)phenyl)-4-(pyridin-4-yl)-1H-imidazol-5-yl)-2,3-dihydroinden-1-one oxime) and AZ628 (3-(2-cyanopropan-2-yl)-N-(4-methyl-3-(3-methyl-4-oxo-3,4-dihydroquinazolin-6-ylamino)phenyl)benzamide). Preferred but non-limiting examples of BRAF-inhibitors are vemurafenib and dabrafenib.

A MEK inhibitor can be an inhibitor of the serine/threonine kinase and/or tyrosine kinase activity of MEK1 (UniProtKB Q02750 (MP2K1_HUMAN)), MEK2 (UniProtKB P36507 (MP2K2_HUMAN)) or both, and may also or alternatively inhibit other MEK proteins, such as MEK5 (UniProtKB Q13163 (MP2K5_HUMAN)). Preferably, a MEK inhibitor inhibits the serine/threonine kinase activity of MEK1, MEK2 or both. Examples of MEK-inhibitors include trametinib, cobimetinib, binimetinib and selumetinib. Preferably, the MEK-inhibitor has a similar mechanism of action as trametinib, which is a preferred but non-limiting example of a MEK-inhibitor.

In one embodiment, the serine/threonine kinase inhibitor in the combination, composition or kit comprises at least one BRAF-inhibitor and at least one MEK-inhibitor, wherein the at least one BRAF-inhibitor is selected from vemurafenib, dabrafenib and a combination thereof, and wherein the MEK-inhibitor is selected from selumetinib and trametinib, and a combination thereof. For example, the combination, composition or kit may comprise dabrafenib and trametinib; vemurafenib and trametinib; dabrafenib, vemurafenib and trametinib; dabrafenib and selumetinib; or vemurafenib and selumetinib. In one embodiment, the cancer or tumor is resistant to one or more serine/threonine kinase inhibitors, e.g., the serine/threonine kinase inhibitor, BRAF inhibitor and/or MEK inhibitor provided in the combination, composition or kit.

In a specific embodiment, the cancer or tumor is melanoma, optionally resistant to the serine/threonine kinase inhibitor, BRAF inhibitor and/or MEK inhibitor provided in the combination, composition or kit.

In one embodiment, the at least one second anti-cancer agent in the combination, kit or composition comprises a chemotherapeutic agent. Preferably, the chemotherapeutic agent is selected from the group consisting of paclitaxel, docetaxel, cisplatin, doxorubicin, etoposide, carboplatin and metformin. In one embodiment, the therapeutic agent is a microtubule-targeting agent, such as, e.g., paclitaxel, docetaxel or vincristine, or a therapeutically active analog or derivative of any thereof. In one embodiment, the at least one therapeutic agent is a taxane, such as paclitaxel, docetaxel or a therapeutically active analog or derivative of paclitaxel or docetaxel. In one embodiment, the cancer or tumor is resistant to one or more chemotherapeutic agents, e.g., the chemotherapeutic agent provided in the combination, composition or kit. In one particular embodiment, the chemotherapeutic agent is paclitaxel, and the cancer is cervical cancer, optionally resistant to paclitaxel. In one particular embodiment, the chemotherapeutic agent is paclitaxel, and the cancer is an NSCLC, optionally resistant to paclitaxel. In one particular embodiment, the cancer is an ovarian cancer, optionally resistant to a taxane and/or a platinum derivative, with paclitaxel and cisplatin being preferred but non-limiting examples of a taxane and a platinum derivative, respectively. In one particular embodiment, the chemotherapeutic agent is paclitaxel, and the cancer is an ovarian cancer, optionally resistant to paclitaxel

In one embodiment, the at least one chemotherapeutic agent in the combination, composition or kit is selected from the group consisting of cisplatin, carboplatin, doxorubicin, etoposide and metformin.

In one embodiment, the PI3K inhibitor in the combination, composition or kit is alpelisib (BYL719).

In one embodiment, the mAb/rTKI in the combination, composition or kit is Cetuximab or MAB391.

The response to the AXL-ADC therapy may be evaluated by a person of skill in the art according to known methods, e.g., the guidelines of the NCCN or ESMO. In a specific embodiment, the evaluation can be based on the following criteria (RECIST Criteria v1.1):

TABLE 1
Definition of Response (RECIST Criteria v1.1)

| | Category | Criteria |
|---|---|---|
| Based on target | Complete | Disappearance of all target lesions. Any pathological |
| lesions | Response (CR) | lymph nodes must have reduction in short axis to <10 mm. |
| | Partial Response | ≥30% decrease in the sum of the LD of target |
| | (PR) | lesions, taking as reference the baseline sum LD. |
| | Stable Disease | Neither sufficient shrinkage to qualify for PR nor |
| | (SD) | sufficient increase to qualify for PD, taking as |
| | | reference the smallest sum of LDs since the treatment |
| | | started. |
| | Progressive | ≥20% increase in the sum of the LDs of target |
| | Disease (PD) | lesions, taking as reference the smallest sum of the |
| | | LDs recorded since the treatment started or the |
| | | appearance of one or more new lesions. |
| Based on non- | CR | Disappearance of all non-target lesions and |
| target lesions | | normalization of tumor marker level. All lymph nodes |
| | | must be non-pathological in size (<10 mm short |
| | | axis). |
| | SD | Persistence of one or more non-target lesion(s) |
| | | or/and maintenance of tumor marker level above the |
| | | normal limits. |
| | PD | Appearance of one or more new lesions and/or |
| | | unequivocal progression of existing non-target |
| | | lesions. |

Pharmaceutical Compositions

In another aspect the invention, the AXL-ADC for use according to any aspect or embodiment of the invention as described herein is comprised in a pharmaceutical composition. In one embodiment the pharmaceutical composition further comprises a pharmaceutically acceptable carrier. In particular, upon purifying the AXL-ADCs they may be formulated into pharmaceutical compositions using well known pharmaceutical carriers or excipients.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutically acceptable carriers or diluents as well as any other known adjuvants and excipients should be suitable for the ADC of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact (10% or less relative inhibition, 5% or less relative inhibition, etc.)) on antigen binding.

A pharmaceutical composition of the present invention may also include diluents, fillers, salts, buffers, detergents (e. g., a nonionic detergent, such as Tween-20 or Tween-80), stabilizers (e. g., sugars or protein-free amino acids), preservatives, tissue fixatives, solubilizers, and/or other materials suitable for inclusion in a pharmaceutical composition.

The pharmaceutical composition may be administered by any suitable route and mode. Suitable routes of administering an antibody drug conjugate of the present invention are well known in the art and may be selected by those of ordinary skill in the art.

In one embodiment, the pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In one embodiment the pharmaceutical composition is administered by intravenous or subcutaneous injection or infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with antibody drug conjugate of the present invention.

Examples of suitable aqueous-and non-aqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the AXL-ADC of the present invention, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The AXL-ADC of the present invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J. R. Robinson, ed., Marcel Dekker, Inc., New York, 1978.

In one embodiment, the AXL-ADC of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the AXL-ADC in the required amount in an appropriate solvent with one or a combination of ingredients e.g. as enumerated above, as required, followed by sterilization microfiltration. Generally, dispersions are prepared by incorporating the AXL-ADC into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the AXL-ADC in the required amount in an appropriate solvent with one or a combination of ingredients enumerated above, as required, followed by sterilization microfiltration. Generally, dispersions are prepared by incorporating the AXL-ADC into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the AXL-ADC plus any additional desired ingredient from a previously sterile-filtered solution thereof.

In one particular embodiment, the AXL-ADC is comprised in a pharmaceutical composition which comprises one or more excipients but is free of surfactant. In one embodiment, the pharmaceutical composition has a pH of about 5 to about 7 and comprises, in aqueous solution:
(a) from about 5 mg/mL to about 30 mg/mL of an anti-AXL ADC and
(b) from about 10 mM to about 50 mM histidine;
(c) from about 30 mM to about 150 mM sucrose or trehalose; and
(d) from about 50 mM to about 300 mM mannitol or glycine.

In a specific embodiment, the pharmaceutical composition has a pH in the range of about 5.5 to about 6.5 and comprises:
(a) from about 9 mg/mL to about 11 mg/mL AXL ADC, such as about 10 mg/mL of the anti-AXL ADC;
(b) from about 20 mM to about 40 mM histidine, such as about 30 mM histidine;
(c) from about 80 mm to about 100 mM sucrose, such as about 88 mM sucrose; and
(d) from about 150 mM to about 180 mM mannitol, such as about 165 mM; and is free of any surfactant.

In another specific embodiment, the pharmaceutical composition has a pH in the range of about 5.5 to about 6.5 and comprises:
(a) about 10 mg/mL of the anti-AXL ADC;
(b) about 30 mM histidine;
(c) about 88 mM sucrose; and
(d) about 165 mM;
   and is free of any surfactant.

TABLE 2
Sequences

| SEQ | | | |
|---|---|---|---|
| ID | | | |
| NO: | Name | Amino acid sequence | Comments |
| 1 | 107 VH | EVQLLESGGGLVQPGGSLRLSCAASGFTFSSYAMNWVRQAPGK | HCo12-BalbC |
| | | GLEWVSTTSGSGASTYYADSVKGRFTISRDNSKNTLYLQMNSLR | Ig1 domain |
| | | AEDTAVYYCAKIWIAFDIWGQGTMVTVSS | binding Ab |
| 2 | 107 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGSSPYTFGQGTKLEIK | |
| 3 | 140 VH | EVQLLESGGGLVQPGGSLRLSCAASGFTFSSYAMTWVRQAPGK | |
| | | GLEWVSAISISGASTFYADSVKGRFTISRDNSKNTLSLQMNSLRA | |
| | | EDTAVYFCRGYSGYVYDAFDIWGQGTMVTVSS | |
| 4 | 140 VL | DIQMTQSPSSLSASVGDRVTITCRASQGISNWLAWYQQKPEKA | |
| | | PKSLIYAASSLQSGVPSRFSGSGSGTDFTLTISSLQPEDFATYYCQ | |
| | | QYNSYPLTFGGGTKVEIK | |
| 5 | 148 VH | EVQLLESGGGLVQPGGSLRLSCAASGFTFSSYAMTWVRQAPGK | HCo12-BalbC |
| | | GLEWVSAISISGGSTFYADSVKGRFTISRDNSKNTLYLQMNSLRA | Ig2 domain |
| | | EDTAVYYCRGYSGYVYDAFDFWGQGTMVTVSS | binding Ab |
| 6 | 148 VL | DIQMTQSPSSLSASVGDRVTITCRASQGISNWLAWYQQKPEKA | |
| | | PKSLIYAASSLQSGVPSRFSGSGSGTDFTLTISSLQPEDFATYYCQ | |
| | | QYNSYPLTFGGGTKVEIK | |
| 7 | 154 VH | EVQLLDSGGGLVQPGGSLRLSCAASGFTFSSYAMSWVRQAPGK | HCo12-BalbC |
| | | GLEWVSAISIGGGNAYYADSVKGRFTISRDNSKNTLYLQMNSLR | FN1 domain |
| | | AADTAVYYCAKPGFIMVRGPLDYWGQGALVTVSS | binding Ab |
| 8 | 154-M103L VH | EVQLLDSGGGLVQPGGSLRLSCAASGFTFSSYAMSWVRQAPGK | |
| | | GLEWVSAISIGGGNAYYADSVKGRFTISRDNSKNTLYLQMNSLR | |
| | | AADTAVYYCAKPGFILVRGPLDYWGQGALVTVSS | |
| 9 | 154 VL | EIVLTQSPGTLSLSPGERATLSCRASQSVSNSYLAWYQQKPGQA | |
| | | PRLLIYGASSRATGIPDRFSGSGSGTDFTLTISRLEPEDFAVYYCQ | |
| | | QYGSSPYTFGQGTKLEIK | |
| 10 | 171 VH | EVQLLESGGGLVQPGGSLRLSCAASGFTFSSYAMSWVRQAPGK | HCo17-BalbC |
| | | GLEWVSDISVSGGSTYYADSVKGRFTISRDNSKNTLYLQMNSLR | Ig2 domain |
| | | AEDTAVYYCAKEGYIWFGESLSYAFDIWGQGTMVTVSS | binding Ab |
| 11 | 171 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGRSFTFGPGTKVDIK | |
| 12 | 172 VH | EVQLLESGGGLVQPGGSLRLSCAASGFTFSNYAMSWVRQAPGK | |
| | | GLEWVSDISVSGGSTYYADSVKGRFTISRDNSKNTLYLQMNSLR | |
| | | AEDTAVYYCAKEGYIWFGESLSYAFDIWGQGTMVTVSS | |
| 13 | 172 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGRSFTFGPGTKVDIK | |
| 14 | 181 VH | EVQLLESGGGLVQPGGSLRLSCAASGFTFSSYAMSWVRQAPGK | |
| | | GLEWVSDISVSGGSTYYADSVKGRFTISRDNSKNTLYLHMNSLR | |
| | | AEDTAVYYCAKEGYIWFGESLSYAFDIWGQGTMVTVSS | |
| 15 | 181 VH | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGRSFTFGPGTKVDIK | |
| 16 | 183 VH | QVQLQQWGAGLLKPSETLSLTCAVYGGSFSGYYWSWIRQPPGK | HCo17-BalbC |
| | | GLEWIGEINQSGSTNYNPSLKSRVTISVDTSKNQFSLKLSSVTAA | FN1 domain |
| | | DTSVYYCASGNWDHFFDYWGQGTLVTVSS | binding Ab |
| 17 | 183-N52Q VH | QVQLQQWGAGLLKPSETLSLTCAVYGGSFSGYYWSWIRQPPGK | |
| | | GLEWIGEIQQSGSTNYNPSLKSRVTISVDTSKNQFSLKLSSVTAA | |
| | | DTSVYYCASGNWDHFFDYWGQGTLVTVSS | |
| 18 | 183 VL | DIQMTQSPSSVSASVGDRVTITCRASQGISSWLAWYQHKPGKA | |
| | | PKLLIYATSSLQSGVTSRFSGSGSGTDFTLTISSLQPEDFATYYCQQ | |
| | | AKSFPWTFGQGTKVEIK | |
| 19 | 187 VH | QVPLQQWGAGLLKPSETLSLTCAVYGGSFSGYHWSWIRQPPGK | |
| | | GLEWIGEISHSGRTNYNPSLKSRVTISIDTSKNQFSLKLSSVTAAD | |
| | | TAVYYCASFITMIRGTIITHFDYWGQGTLVTVSS | |
| 20 | 187 VL | DIQMTQSPSSLSASVGDRVTITCRASQGISSWLAWYQQKPEKA | |
| | | PKSLIYAASSLQSGVPSRFSGSGSGTDFTLTISSLQPEDFATYYCQ | |
| | | QYHSYPYTFGQGTKLEIK | |
| 21 | 608-01 VH | QVQLVQSGAEVKKPGSSVKVSCKASGGTFSSYAISWVRQAPGQ | |
| | | GLEWMGRIIPIFGIANYVQKFQGRVTITADKSTSTAYMELSSLRA | |
| | | EDTAVYYCARRGDYYGSGSPDVFDIWGQGTMVTVSS | |
| 22 | 608-01 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGSSYTFGQGTKLEIK | |
| 23 | 610-01 VH | QBQLVQSGAEVKKPGSSVKVSCKASGGTFSSYAISWVRQAPGQ | |
| | | GLEWMGRIIPIFGIANYVQKFQGRVTITADKSTSTAYMELSSLRA | |
| | | EDTAVYYCARRGNYYGSGSPDVFDIWGQGTMVTVSS | |
| 24 | 610-01 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGSSYTFGQGTKLEIK | |
| 25 | 613 VH | QVQLVQSGAEVKKPGSSVKVSCKASGGTFSSYAINWMRQAPG | HCo20 |
| | | QGLEWMGRIIPIFGIVNYAQKFQGRVTLTADKSTSTAYMELSSLR | Ig1 domain |
| | | SEDTAVYYCARRGNYYGSGSPDVFDIWGQGTMVTVSS | binding Ab |
| 26 | 613 VL | EIVLTQSPGTLSLSPGERATLSCRASQSVSSSYLAWYQQK | |
| | | PGQAPRLLIYGASSRATGIPDRFSGSGSGTDFTLTISRLEPE | |
| | | DFAVYYCQQYGSSYTFGQGTKLEIK | |
| 27 | 613-08 VH | QVQLVQSGAEVKKPGSSVKVSCKASGGTFSSYAINWMRQAPG | |
| | | QGLEWMGRIIPIFGIVNYAQKFQGRVTLTADKSTSTAYMELSSLR | |
| | | SEDTAVYYCARRGNYYGSGSPDVFDIWGQGTMVTVSS | |
| 28 | 613-08 VL | EIVLTQSPATLSLSPGERATLSCRASQSVSSYLAWYQQKPGQAPR | |
| | | LLIYDASNRATGIPARFSGSGSGTDFTLTISSLEPEDFAVYYCQQR | |
| | | SNWLTFGGGTKVEIK | |
| 29 | 620-06 VH | QVQLVQSGAEVKKPGSSVKVSCKASGGTFSSYAISWVRQAPGQ | |
| | | GLEWMGRIIPIFGIANYAQKFQGRVTITADKSTSTAYMELSSLRS | |
| | | EDTAVYYCARRGNYYGSGSPDVFDIWGQGTMVTVSS | |
| 30 | 620-06 VL | EIVLTQSPGTLSLSPGERATLSCRASQSVSSSYLAWYQQKPGQAP | |
| | | RLLIYGASSRATGIPDRFSGSGSGTDFTLTISRLEPEDFAVYYCQQ | |
| | | YGSSYTFGQGTKLEIK | |
| 31 | 726 VH | QVQLQQWGAGLLKPSETLSLTCAIDGGSFSGYYWSWIRQPPGK | HCo17-BalbC |
| | | GLEWIGEISHSGRTNYNPSLKSRVTISIDTSKNQFSLKLSSVAAAD | FN2 domain |
| | | TAVYYCARFITMIRGAIITHFDYWGQGALVTVSS | binding Ab |
| 32 | 726-M101L VH | QVQLQQWGAGLLKPSETLSLTCAIDGGSFSGYYWSWIRQPPGK | |
| | | GLEWIGEISHSGRTNYNPSLKSRVTISIDTSKNQFSLKLSSVAAAD | |
| | | TAVYYCARFITLIRGAIITHFDYWGQGALVTVSS | |
| 33 | 726 VL | DIQMTQSPSSLSASVGDRVTITCRASQGISSWLAWYQQKPEKA | |
| | | PKSLYAASSLQSGVPSRFSGSGSGTDFTLTISSLQPEDFATYYCQ | |
| | | QYHSYPYTFGQGTKLEIK | |
| 34 | 733 VH | QVQLVESGGGVVQPGRSLRLSCAASGFSFSTYAMHWVRQAPG | HCo17-BalbC |
| | | KGLEWVAVISYDGDNKYSADSVKGRFTISRDNSKNTLYLQMNSL | FN1 domain |
| | | RAEDTAVYYCARGRKLGIDAFDIWGQGTMVTVSS | binding Ab |
| 35 | 733 VL | AIQLTQSPSSLSASVGDRVTITCRASQGISSALAWYQQKPGKAPK | |
| | | LLIYDASSLESGVPSRFSGSGSGTDFTLTISGLQPEDFATYYCQQF | |
| | | NSYPFTFGPGTKVDIK | |
| 36 | 107 VH CDR1 | GFTFSSYA | |
| 37 | 107 VH CDR2 | TSGSGAST | |
| 38 | 107 VH CDR3 | AKIWIAFDI | |
| 39 | 107 VL CDR1 | QSVSSSY | |
| | 107 VL CDR2 | GAS | |
| 40 | 107 VL CDR3 | QQYGSSPYT | |
| 41 | 140 VH CDR1 | GFTFSSYA | |
| 42 | 140 VH CDR2 | ISISGAST | |
| 43 | 140 VH CDR3 | RGYSGYVYDAFDI | |
| 44 | 140 VL CDR1 | QGISNW | |
| | 140 VL CDR2 | AAS | |
| 45 | 140 VL CDR3 | QQYNSYPLT | |
| 46 | 148 VH CDR1 | GFTFSSYA | |
| 47 | 148 VH CDR2 | ISISGGST | |
| 48 | 148 VH CDR3 | RGYSGYVYDAFDF | |
| 49 | 148 VL CDR1 | QGISNW | |
| | 148 VL CDR2 | AAS | |
| 50 | 148 VL CDR3 | QQYNSYPLT | |
| 51 | 154 VH CDR1 | GFTFSSYA | |
| 52 | 154 VH CDR2 | ISIGGGNA | |
| 53 | 154 VH CDR3 | AKPGFIMVRGPLDY | |
| 54 | 154-M103L VH | AKPGFILVRGPLDY | |
| | CDR3 | | |
| 55 | 154 VL CDR1 | QSVSNSY | |
| | 154 VL CDR2 | GAS | |
| 56 | 154 VL CDR3 | QQYGSSPYT | |
| 57 | 171 VH CDR1 | GFTFSSYA | |
| 58 | 171 VH CDR2 | ISVSGGST | |
| 59 | 171 VH CDR3 | AKEGYIWFGESLSYAFDI | |
| 60 | 171 VL CDR1 | QSVSSSY | |
| | 171 VL CDR2 | GAS | |
| 61 | 171 VL CDR3 | QQYGRSFT | |
| 62 | 172 VH CDR1 | GFTFSNYA | |
| 63 | 172 VH CDR2 | ISVSGGST | |
| 64 | 172 VH CDR3 | AKEGYIWFGESLSYAFDI | |
| 65 | 172 VL CDR1 | QSVSSSY | |
| | 172 VL CDR2 | GAS | |
| 66 | 172 VL CDR3 | QQYGRSFT | |
| 67 | 181 VH CDR1 | GFTFSSYA | |
| 68 | 181 VH CDR2 | ISVSGGST | |
| 69 | 181 VH CDR3 | AKEGYIWFGESLSYAFDI | |
| 70 | 181 VL CDR1 | QSVSSSY | |
| | 181 VL CDR2 | GAS | |
| 71 | 181 VL CDR3 | QQYGRSFT | |
| 72 | 183 VH CDR1 | GGSFSGYY | |
| 73 | 183 VH CDR2 | INQSGST | |
| 74 | 183-N52Q VH | IQQSGST | |
| | CDR2 | | |
| 75 | 183 VH CDR3 | ASGNWDHFFDY | |
| 76 | 183 VL CDR1 | QGISSW | |
| | 183 VL CDR2 | ATS | |
| 77 | 183 VL CDR3 | QQAKSFPWT | |
| 78 | 187 VH CDR1 | GGSFSGYH | |
| 79 | 187 VH CDR2 | ISHSGRT | |
| 80 | 187 VH CDR3 | ASFITMIRGTIITHFDY | |
| 81 | 187 VL CDR1 | QGISSW | |
| | 187 VL CDR2 | AAS | |
| 82 | 187 VL CDR3 | QQYHSYPYT | |
| 83 | 608-01 VH CDR1 | GGTFSSYA | |
| 84 | 608-01 VH CDR2 | IIPIFGIA | |
| 85 | 608-01 VH CDR3 | ARRGDYYGSGSPDVFDI | |
| 86 | 608-01 VL CDR1 | QSVSSSY | |
| | 608-01 VL CDR2 | GAS | |
| 87 | 608-01 VL CDR3 | QQYGSSYT | |
| 88 | 610-01 VH CDR1 | GGTFSSYA | |
| 89 | 610-01 VH CDR2 | IIPIFGIA | |
| 90 | 610-01 VH CDR3 | ARRGNYYGSGSPDVFDI | |
| 91 | 610-01 VL CDR1 | QSVSSSY | |
| | 610-01 VL CDR2 | QQYGSSYT | |
| 92 | 610-01 VL CDR3 | QQYGSSYT | |
| 93 | 613 VH CDR1 | GGTFSSYA | |
| 94 | 613 VH CDR2 | IIPIFGIV | |
| 95 | 613 VH CDR3 | ARRGNYYGSGSPDVFDI | |
| 96 | 613 VL CDR1 | QSVSSSY | |
| | 613 VL CDR2 | GAS | |
| 97 | 613 VL CDR3 | QQYGSSYT | |
| 98 | 613-08 VH CDR1 | GGTFSSYA | |
| 99 | 613-08 VH CDR2 | IIPIFGIV | |
| 100 | 613-08 BH CDR3 | ARRGNYYGSGSPDVFDI | |
| 101 | 613-08 VL CDR1 | QSVSSY | |
| | 613-08 VL CDR2 | DAS | |
| 102 | 613-08 VL CDR3 | QQRSNWLT | |
| 103 | 620-06 VH CDR1 | GGTFSSYA | |
| 104 | 620-06 VH CDR2 | IIPIFGIA | |
| 105 | 620-06 VH CDR3 | ARRGNYYGSGSPDVFDI | |
| 106 | 620-06 VL CDR1 | QSVSSSY | |
| | 620-06 VL CDR2 | GAS | |
| 107 | 620-06 VL CDR3 | QQYGSSYT | |
| 108 | 726 VH CDR1 | GGSFSGYY | |
| 109 | 726 VH CDR2 | ISHSGRT | |
| 110 | 726 VH CDR3 | ARFITMIRGAIITHFDY | |
| 111 | 726-m101L VH | ARFITLIRGAIITHFDY | |
| | CDR3 | | |
| 112 | 726 VL CDR1 | QGISSW | |
| | 726 VL CDR2 | AAS | |
| 113 | 726 VL CDR3 | QQYHSYPYT | |
| 114 | 733 VH CDR1 | GFSFSTYA | |
| 115 | 733 VH CDR2 | ISYDGDNK | |
| 116 | 733 VH CDR3 | ARGRKLGIDAFDI | |
| 117 | 733 VL CDR1 | QGISSA | |
| | 733 VL CDR2 | DAS | |
| 118 | 733 VL CDR3 | QQFNSYPFT | |
| 119 | Ig2 domain VH | ISISGXST-wherein X is A or G | |
| | CDR2 | | |
| 120 | Ig2 domain VH | RGYSGYVYDAFDX-wherein X is I or F | |
| | CDR3 | | |
| 121 | FN2 domain VH | GGSFSGYX-wherein X is H or Y | |
| | CDR1 | | |
| 122 | FN2 domain VH | AX1FITMIRGX2IITHFDY-wherein X1 is S or R; and X2 is T | |
| | CDR3 | or A | |
| 123 | FN1 domain VH | GFTFSXYA-wherein X is S or N | |
| | CDR1 | | |
| 124 | FN1 domain VH | ISVSGGST | |
| | CDR2 | | |
| 125 | FN1 domain VH | AKEGYIWFGESLSYAFDI | |
| | CDR3 | | |
| 126 | Ig1 domain VH | IIPIFGIX-wherein X is A or V | |
| | CDR2 | | |
| 127 | Ig1 domain VH | ARRGXYYGSGSPDVFDI-wherein X is D or N | |
| | CDR3 | | |
| 128 | Ig1 domain VL | QSVXSSY-wherein X is S or del | |
| | CDR1 | | |
| | Ig1 domain VL | XAS-wherein X is D or G | |
| | CDR2 | | |
| 129 | Ig1 domain VL | QQX1X2X3X4X5T-wherein X1 is R or Y;X2 is S or G; X3 is | |
| | CDR3 | N or S; X4 is W or S; and X5 is L or Y | |
| 130 | Human AXL | MAWRCPRMGRVPLAWCLALCGWACMAPRGTQAEESPFVGN | |
| | protein | PGNITGARGLTGTLRCQLQVQGEPPEVHWLRDGQILELADSTQT | |
| | (Swissprot | QVPLGEDEQDDWIVVSQLRITSLQLSDTGQYQCLVFLGHQTFVS | |
| | P30530) | QPGYVGLEGLPYFLEEPEDRTVAANTPFNLSCQAQGPPEPVDLL | |
| | | WLQDAVPLATAPGHGPQRSLHVPGLNKTSSFSCEAHNAKGVTT | |
| | | SRTATITVLPQQPRNLHLVSRQPTELEVAWTPGLSGIYPLTHCTL | |
| | | QAVLSDDGMGIQAGEPDPPEEPLTSQASVPPHQLRLGSLHPHT | |
| | | PYHIRVACTSSQGPSSWTHWLPVETPEGVPLGPPENISATRNGS | |
| | | QAFVHWQEPRAPLQGTLLGYRLAYQGQDTPEVLMDIGLRQEVT | |
| | | LELQGDGSVSNLTVCVAAYTAAGDGPWSLPVPLEAWRPGQAQ | |
| | | PVHQLVKEPSTPAFSWPWWYVLLGAVVAAACVLILALFLVHRRK | |
| | | KETRYGEVFEPTVERGELVVRYRVRKSYSRRTTEATLNSLGISEELK | |
| | | EKLRDVMVDRHKVALGKTLGEGEFGAVMEGQLNQDDSILKVA | |
| | | VKTMKIAICTRSELEDFLSEAVCMKEFDHPNVMRLIGVCFQGSER | |
| | | ESFPAPVVILPFMKHGDLHSFLLYSRLGDQPVYLPTQMLVKFMA | |
| | | DIASGMEYLSTKRFIHRDLAARNCMLNENMSVCVADFGLSKKIY | |
| | | NGDYYRQGRIAKMPVKWIAIESLADRVYTSKSDVWSFGVTMW | |
| | | EIATRGQTPYPGVENSEIYDYLRQGNRLKQPADCLDGLYALMSR | |
| | | CWELNPQDRPSFTELREDLENTLKALPPAQEPDEILYVNMDEGG | |
| | | GYPEPPGAAGGADPPTQPDPKDSCSCLTAAEVHPAGRYVLCPST | |
| | | TPSPAQPADRGSPAAPGQEDGA | |
| 131 | Mus musculus | MAWRCPRMGRVPLAWCLALCGWACMYPYDVPDYAAHKDTQ | |
| | AXL | TEAGSPFVGNPGNITGARGLTGTLRCELQVQGEPPEVVWLRDG | |
| | | QILELADNTQTQVPLGEDWQDEWKVVSQLRISALQLSDAGEYQ | |
| | | CMVHLEGRTFVSQPGFVGLEGLPYFLEEPEDKAVPANTPFNLSC | |
| | | QAQGPPEPVTLLWLQDAVPLAPVTGHSSQHSLQTPGLNKTSSFS | |
| | | CEAHNAKGVTTSRTATITVLPQRPHHLHVVSRQPTELEVAWTPG | |
| | | LSGIYPLTHCNLQAVLSDDGVGIWLGKSDPPEDPLTLQVSVPPH | |
| | | QLRLEKLLPHTPYHIRISCSSSQGPSPWTHWLPVETTEGVPLGPP | |
| | | ENVSAMRNGSQVLVRWQEPRVPLQGTLLGYRLAYRGQDTPEV | |
| | | LMDIGLTREVTLELRGDRPVANLTVSVTAYTSAGDGPWSLPVPL | |
| | | EPWRPGQGQPLHHLVSEPPPRAFSWPWWYVLLGAVVAAACV | |
| | | LILALFLVHRRKKETRYGEVFEPTVERGELVVRYRVRKSYSRRTTE | |
| | | ATLNSLGISEELKEKLRDVMVDRHKVALGKTLGEGEFGAVMEGQ | |
| | | LNQDDSILKVAVKTMKIAICTRSELEDFLSEAVCMKEFDHPNVM | |
| | | RLIGVCFQGSERESFPAPVVILPFMKHGDLHSFLLYSRLGDQPVYL | |
| | | PTQMLVKFMADIASGMEYLSTKRFIHRDLAARNCMLNENMSV | |
| | | CADFGLSKKIYNGDYYRQGRIAKMPVKWIAIESLADRVYTSKSD | |
| | | VWSFGVTMWEIATRGQTPYPGVENSEIYDYLRQGNRLKQPADC | |
| | | LDGLYALMSRCWELNPQDRPSFTELREDLENTLKALPPAQEPDEI | |
| | | LYVNMDEGGGYPEPPGAAGGADPPTQPDPKDSCSCLTAAEVH | |
| | | PAGRYVLCPSTTPSPAQPADRGSPAAPGQEDGA | |
| 136 | 511 VH | EVQLLESGGGLVQPGGSLRLSCAASGFTFSSYAMNWVRQAPGK | Ig2 domain |
| | | GLEWVSGISGSGGHTYHADSVKGRFTISRDNSKNTLYLQMNSLR | binding Ab |
| | | AEDTAVYYCAKDRYDILTGYYNLLDYWGQGTLVTVSS | |
| 137 | 511 VH CDR1 | GFTFSSYA | |
| 138 | 511 VH CDR2 | ISGSGGHT | |
| 139 | 511 VH CDR3 | AKDRYDILTGYYNLLDY | |
| 140 | 511 VL | DIQMTQSPSSLSASVGDRVTITCRASQGISSWLAWYQQKPEEAP | |
| | | KSLIYAASSLQSGVPSRFSGSGSGTDFTLTISSLQPEDFATYYCQQ | |
| | | YNSYPLTFGGGAKVEIK | |
| 141 | 511 VL CDR1 | QGISSW | |
| | 511 VL CDR2 | AAS | |
| 142 | 511 VL CDR3 | QQYNSYPLT | |
| 143 | 061 VH | QVQLVQSGAEVKKPGASVKVSCKASGYAFTGYGISWVRQAPGQ | Ig1 domain |
| | | GLEWIGWISAYNGNTNYVQNLQDRVTMTTDTSTSTAYMELRSL | binding Ab |
| | | RSDDTAVYYCARDHISMLRGIIIRNYWGQTLVTVSS | |
| 144 | 061 VL | EIVLTQSPATLSLSPGERATLSCRASQSVSSYLAWYQQKPGQAPR | |
| | | LLIYDASNRATGIPARFSGSGSGTDFTLTISSLEPEDFAVYYCQQRS | |
| | | SWPRLTFGGGTKVEIK | |
| 145 | 137 VH | QVQLVQSGAEVKKPGSSVKVSCKASGGTFSRYAISWVRQAPGQ | |
| | | GLEWMGRIIPIVGIANYAQKFQGRVTLTADKSTSTAYMELSSLRS | |
| | | EDTAVYYCAREAGYSSSWYAEFQHWGQGTLVTVSS | |
| 146 | 137 VL | EIVLTQSPGTLSLSPGERATLSCRASQSVSSNYLAWYQQKPGQAP | |
| | | RLLIYGASSRATFGPDRFSGSGSGTDFTLTISRLEPEDFAVYYCQQ | |
| | | YGSSPYTFGQGTKLEIK | |
| 147 | Cynomolgus | AWRCPRMGRVPLAWCLALCGWVCMAPRGTQAEESPFVGNP | |
| | monkey AXL | GNITGARGLTGTLRCQLQVQGEPPEVHWLRDGQILELADSTQT | |
| | (GenBank | QVPLGEDEQDDWIVVSQLRIASLQLSDAGQYQCLVFLGHQNFV | |
| | number | SQPGYVGLEGLPYFLEEPEDRTVAANTPFNLSCQAQGPPEPVDL | |
| | HB387229.1) | LWLQDAVPLATAPGHGPQRNLHVPGLNKTSSFSCEAHNAKGVT | |
| | | TSRTATITVLPQQPRNLHLVSRQPTELEVAWTPGLSGIYPLTHCTL | |
| | | QAVLSDDGMGIQAGEPDPPEEPLTLQASVPPHQLRLGSLHPHTP | |
| | | YHIRVACTSSQGPSSWTHWLPVETPEGVPLGPPENISATRNGSQ | |
| | | AFVHWQEPRAPLQGTLLGYRLAYQGQDTPEVLMDIGLRQEVTL | |
| | | ELQGDGSVSNLTVCVAAYTAAGDGPWSLPVPLEAWRPGQAQP | |
| | | VHQLVKETSAPAFSWPWWYILLGAVVAAACVLILALFLVHRRKK | |
| | | ETRYGEVFEPTVERGELVVRYRVRKSYSRRTTEATLNSLGISEELKE | |
| | | KLRDVMVDRHKVALGKTLGEGEFGAVMEGQLNQDDSILKVAV | |
| | | KTMKIAICTRSELEDFLSEAVCMKEFDHPNVMRLIGVCFQGSERE | |
| | | SFPAPVVILPFMKHGDLHSFLLYSRLGDQPVYLPTQMLVKFMAD | |
| | | IASGMEYLSTKRFIHRDLAARNCMLNENMSVCVADFGLSKKIYN | |
| | | GDYYRQGRIAKMPVKWIAIESLADRVYTSKSDVWSFGVTMWEI | |
| | | ATRGQTPYPGVENSEIYDYLRQGNRLKQPADCLDGLYALMSRC | |
| | | WELNPQDRPSFTELREDLENTLKALPPAQEPDEILYVNMDEGGG | |
| | | YPEPPGAAGGADPPTQLDPKDSCSCLTSAEVHPAGRYVLCPSTA | |
| | | PSPAQPADRGSPAAPGQEDGA | |

The present invention is further illustrated by the following Examples which should not be construed as further limiting.

### EXAMPLES

### Example 1

### Population Pharmacokinetic (popPK) Model

This Example estimates the exposure of free HuMax-AXL-ADC (IgG1-1021-107-MMAE) and free MMAE in plasma for humans for the following two dosing regimens:
1Q3W: Dosing once every 3 weeks (0.3, 0.6, 1, 1.5, 2, 2.4 mg/kg).
3Q4W: Weekly dosing for 3 weeks followed by one treatment-free week (0.6, 0.8, 1, 1.2, 1.4 mg/kg).

This was done by first developing a population PK model for cynomolgus monkeys using all the available pre-clinical data, before scaling it allometrically to humans. Simulations of the above dosing regimens and levels were conducted in order to determine the following exposure parameters for each case: the maximal plasma concentration Cₘₐₓ, the time of the maximal MMAE exposure Tₘₐₓ, the area under the concentration curve (AUC) over the first cycle, AUC over the 3^{rd} cycle, AUC over 12 weeks of treatment, the lowest plasma concentration observed during the 2^{nd} cycle (C_{trough}) and the terminal half-life of the drug.

Data

Data from 5 in-vivo pre-clinical studies, shown in the Table below, were provided, containing measurements of the plasma ADC level, plasma IgG level, and plasma MMAE level (only measured in study 5).

TABLE 3
Pre-clinical studies in cynomolgus monkey

| | | | Day(s) of |
|---|---|---|---|
| Study no. | Treatment groups | N | administration |
| 1 | IgG1-1021-107-MMAE (HuMax-AXL- | 2 (1M, 1F) | Day 1, day 22 |
| | ADC), 3 mg/kg | | |
| | IgG1-1021-107-MMAE (HuMax-AXL- | 2 (1M, 1F) | Day 1, day 22 |
| | ADC), 4.5 mg/kg | | |
| | IgG1-1021-733-MMAE, 3 mg/kg | 2 (1M, 1F) | Day 1, day 22 |
| | IgG1-1021-733-MMAE, 4.5 mg/kg | 2 (1M, 1F) | Day 1, day 22 |
| 2 | IgG1-1021-148-MMAE, 1 mg/kg | 2 (1M, 1F) | Day 1, day 22, |
| | | | day 43 |
| | IgG1-1021-148-MMAE, 3 mg/kg | 2 (1M, 1F) | Day 1, day 22, |
| | | | day 43 |
| | IgG1-1021-148-MMAE, 4.5 mg/kg | 2 (1M, 1F) | Day 15, day 36, |
| | | | day 57 |
| 3 | IgG1-1021-148-MMAE, 1 mg/kg and | 4 (2M, 2F) | Day 1 |
| | 10 mg/kg | | (1 mg/kg), day |
| | | | 15 (10 mg/kg) |
| 4 | IgG1-1021-107-MMAE (HuMax-AXL- | 2 (1M, 1F) | Day 1, day 22, |
| | ADC), 3 mg/kg | | day 43 |
| | IgG1-1021-107-MMAE (HuMax-AXL- | 2 (1M, 1F) | Day 1, day 22, |
| | ADC), 4.5 mg/kg | | day 43 |
| | IgG1-1021-107-MMAE (HuMax-AXL- | 2 (1M, 1F) | Day 1, day 22, |
| | ADC), 6 mg/kg | | day 43 |
| | IgG1-1021-107-MMAE (HuMax-AXL- | 2 (1M, 1F) | Day 22, day 43 |
| | ADC), 6 mg/kg | | |
| 5 | IgG1-1021-107-MMAE (HuMax-AXL- | 10 (5M, 5F) | Day 1, day 22, |
| | ADC), 1 mg/kg | | day 43 |
| | IgG1-1021-107-MMAE (HuMax-AXL- | 10 (5M, 5F) | Day 1, day 22, |
| | ADC), 3 mg/kg | | day 43 |
| | IgG1-1021-107-MMAE (HuMax-AXL- | 10 (5M, 5F) | Day 1, day 22, |
| | ADC), 5 mg/kg | | day 43 |
| | IgG1-1021-107-MMAE (HuMax-AXL- | 6 (3M, 3F) | Day 1 (Day 1, |
| | ADC), 5 mg/kg | | single dose) |

For all in vivo studies except study 5, IgG1-lambda and IgM antibody-drug antibody (ADA) responses were also provided.

Experimental PK data were generated in cynomolgus monkeys using IgG1-1021-107-MMAE (HuMax-AXL-ADC) and another Axl-specific ADC, IgG1-1021-148-MMAE. IgG1-1021-107-MMAE and IgG1-1021-148-MMAE were found to have comparable functional characteristics, including target binding affinity, internalization characteristics and lack of competition with the Axl ligand Gas6.

In addition, experimental pharmacokinetic data obtained using unconjugated IgG1-1021-148 were included, in order to have the largest possible data set to support the popPK model. The pharmacokinetic properties of unconjugated IgG1-1021-148 (study 3) and IgG1-1021-148-MMAE (study 2) appeared to be similar based on comparison of PK and PK/TK data from studies 3 and study 2, respectively.

All studies appeared to show a significant drop in plasma concentrations of ADC/IgG after the 2^{nd} dose when compared to the first dose, which corresponded with an ADA response. As this is a fully human antibody, ADA response are only very rarely expected to occur in humans. Therefore, only data after the first dose were included in the data preparation and subsequently modelled.

A portion of the ADC, IgG and MMAE data were labelled as below the limit of quantification (BLQ).

### METHODS

Estimation of individual PK parameters was done using NONMEM version 7.3 (Beal et al., NONMEM User's Guides. (1989-2009), Icon Development Solutions, Ellicott City, Md., USA, 2009). Estimation of the exposure parameters and all other preparation, analysis and visualisation of data was done using R version 3.3.1 (Development Core Team (2008). R: A language and environment for statistical computing. R Foundation for Statistical Computing, Vienna, Austria. ISBN 3-900051-07-0).

A population PK model of antibody plasma exposure was developed, identifying a structural model that was able to simultaneously account for antibody exposure as measured by the ADC and IgG assays. The model was developed according to predetermined rules until model bias was minimised or eliminated. Once the model was developed, the individual model fits were used to model MMAE exposure using the same modelling procedure. Throughout modelling, data below the limit of quantification was taken into account using the method of Bergstrand et al. (AAPS journal 2009 June; 11(2): 371-380) and the importance sampling (IMP) method in NONMEM was used for model fitting.

In order to verify the suitability of the model during development, goodness of fit (GOF) plots and prediction correct visual predictive checks (pcVPC) were generated. In addition to this, individual model fits of each cynomolgus monkey were plotted in order to verify the ability of the model to estimate individual profiles.

In order to simulate and estimate the ADC and MMAE exposure in humans, the preclinical ADC and MMAE models were scaled according to the commonly accepted methods of allometry (Kagan et al., Pharm Res (2010) 27:920-932). Rates and volumes were scaled using bodyweight.

For the ADC/IgG model, inter-individual variability (IIV) on the clearance parameters were assumed to be the same in humans as cynomolgus monkeys. IIV was added onto human volume according to Dirks and Meibohm (Clin Pharmacokinet (2010) 49 (19):633-659). Modelling the ADC exposure revealed a study effect on the inter-compartmental clearance parameter Q. During simulation, the value of inter-compartmental clearance for each virtual patient was randomly selected from the 2 values found during modelling.

The bodyweights of the virtual human patients during simulation were sampled from a normal distribution with mean 70 kg and standard deviation 10 kg.

A simulation of 100 virtual patients was done for each dosing level, and the 5^{th}, median and 95^{th} percentiles of the profiles were calculated. The simulations and calculation of the various exposure parameters were done in the software R (Version 3.1.1). For the integration of ODE the package deSolve version 1.11 was used (Soetaert et al. (J Statistical Software (2010) 33(9), pp. 1-25).

### RESULTS

ADC/IgG popPK Model in Cynomolgus Monkeys:

Several structural models were tested in order to model ADC exposure as quantified by the

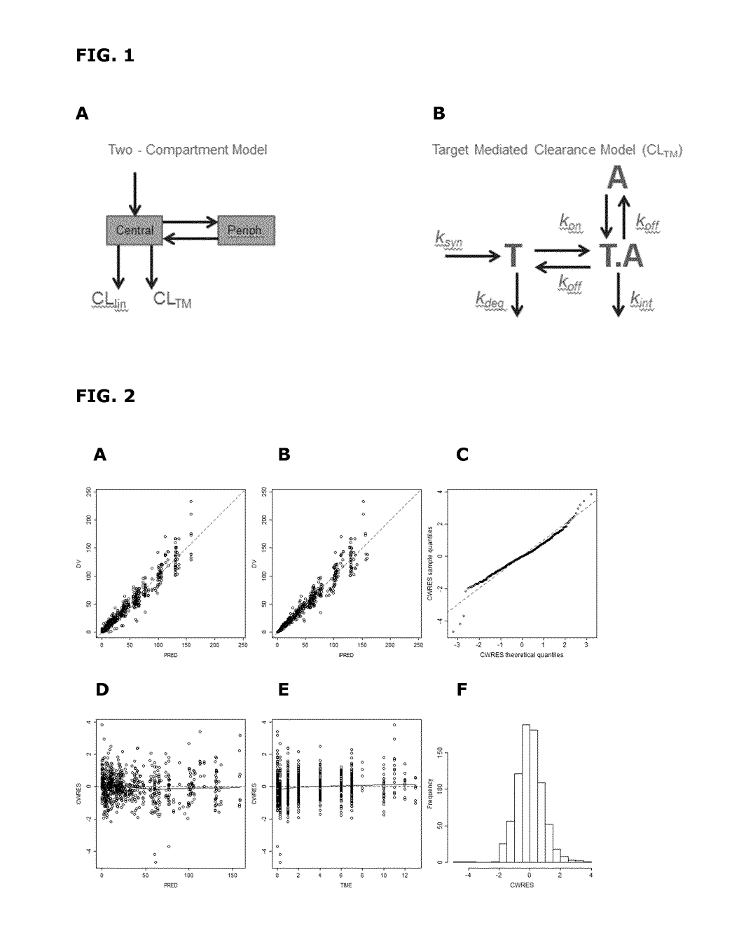

ADC and IgG assay. A two-compartment model which exhibits both target mediated as well as non-target mediated clearance (FIG. 1) was selected as it marked a clear improvement over less complex models. The selected model exhibited a model structure, which is commonly used for describing the exposure to monoclonal antibodies and it has for instance been presented in Gibiansky et al. (J Pharmacokinet Pharmacodyn 2008;35(5):573-91). Allometric scaling of all rate and volume parameters was included in the preclinical model.

Individual trajectories were generated, revealing that the model was well capable of capturing the exposure of the antibody as measured by both IgG as well as ADC assay. Overall, the model proved to be adequate as it described the observed data in an un-biased fashion. FIG. 2 shows the standard GOF plots for the ADC exposure model, which does not exhibit any obvious bias. A prediction-corrected VPC (visual predictive check) of the ADC data was prepared (not shown), where again a good model fit without any obvious bias was observed.

MMAE popPK Model in Cynomolgus Monkeys:

A 2-compartment model proved to be the best fit for modelling the MMAE exposure data from study 5. Allometric scaling by bodyweight was included on all relevant parameters. Following Lu et al. (Pharm Res 2015;32:1907-19), absorption of free MMAE in plasma was modelled as being proportional to the clearance of ADC.

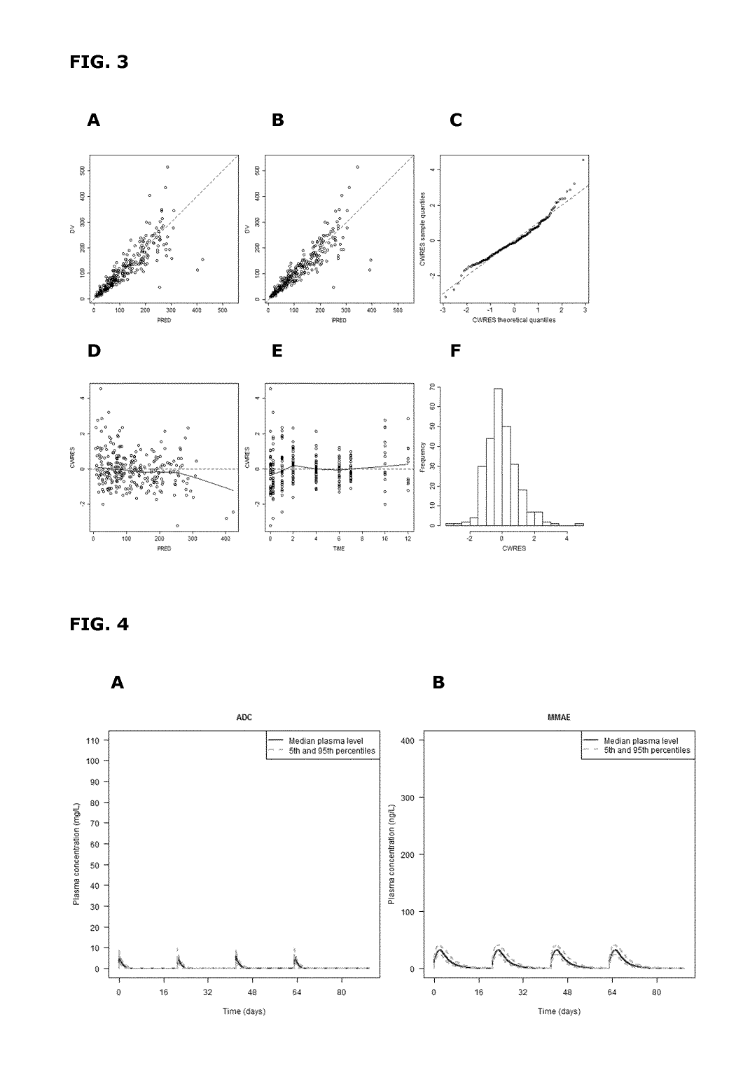

Overall, the MMAE model proved to yield a good and unbiased account of the data, although the confidence interval for one parameter, k_{D2}, was too large. Both the standard GOF plots (FIG. 3) and the prediction corrected VPC (not shown) showed good, unbiased results

Estimation of Human ADC and MMAE Exposure Parameters:

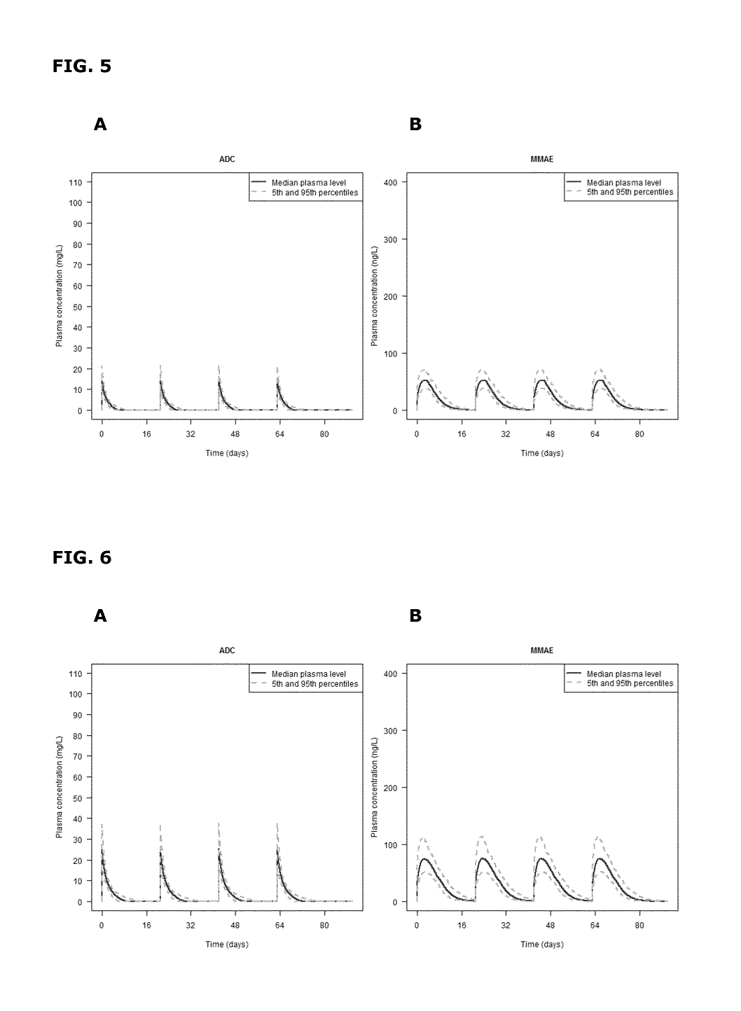

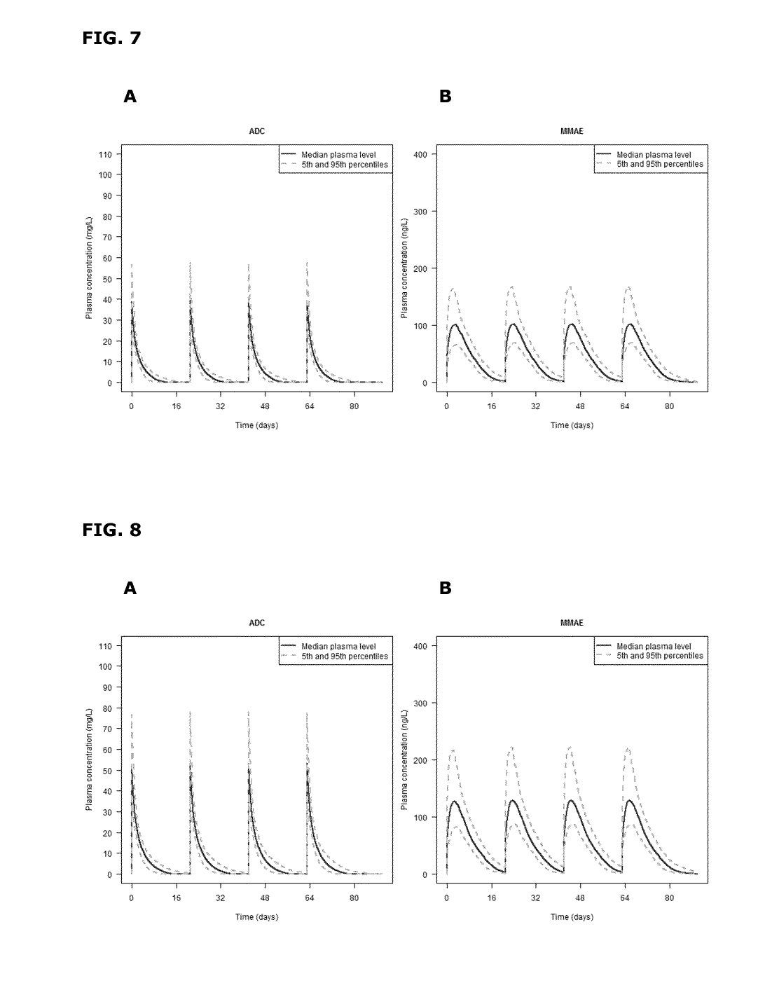

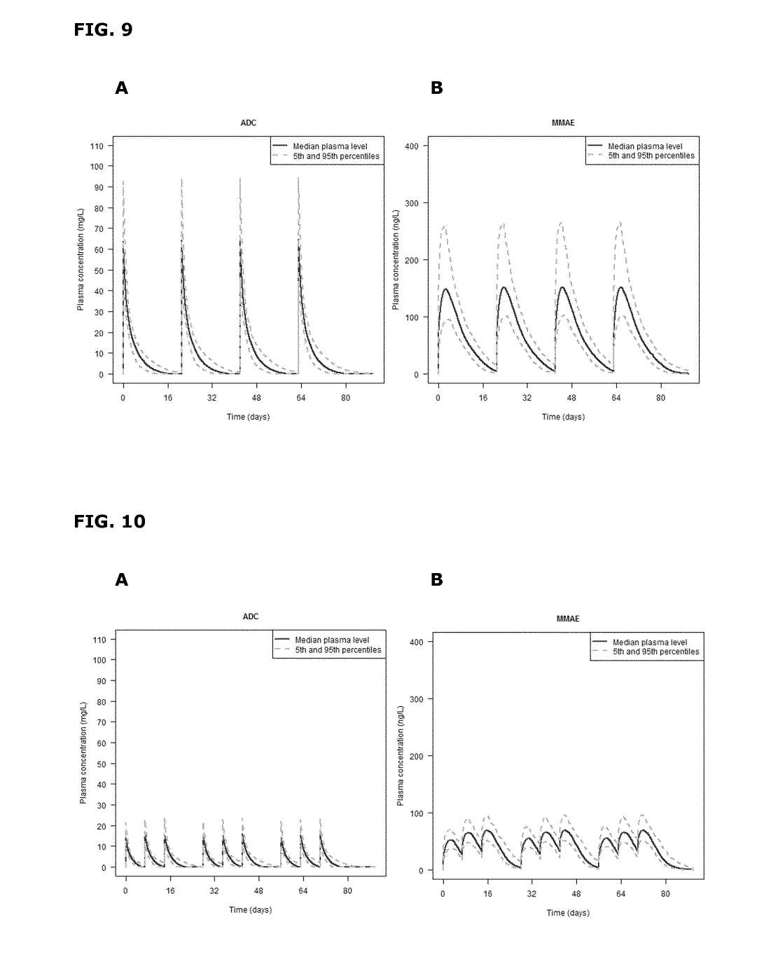

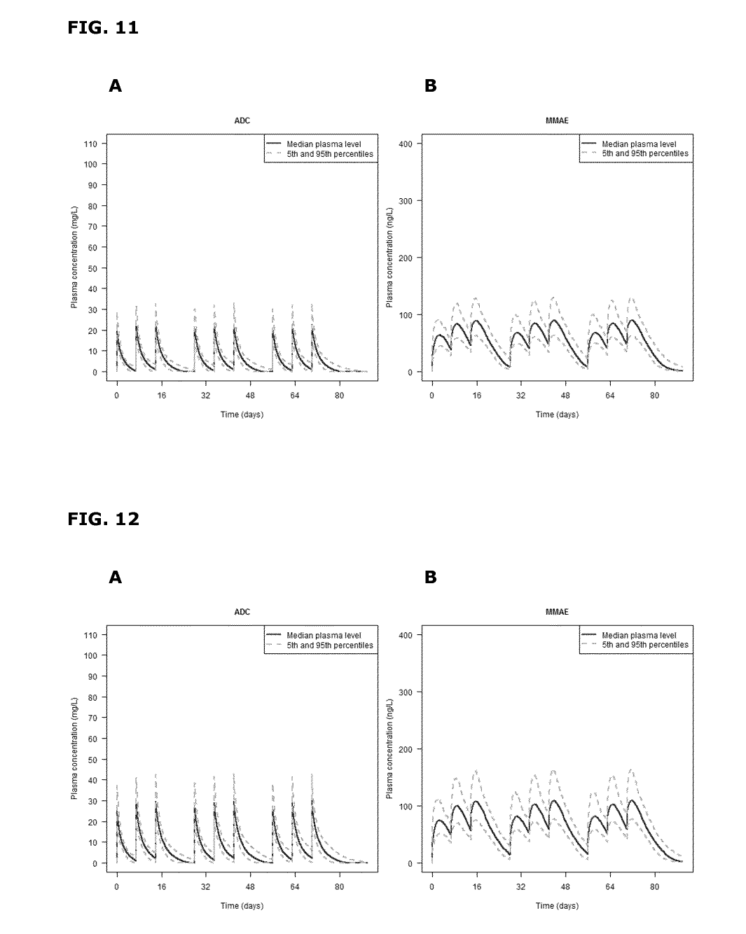

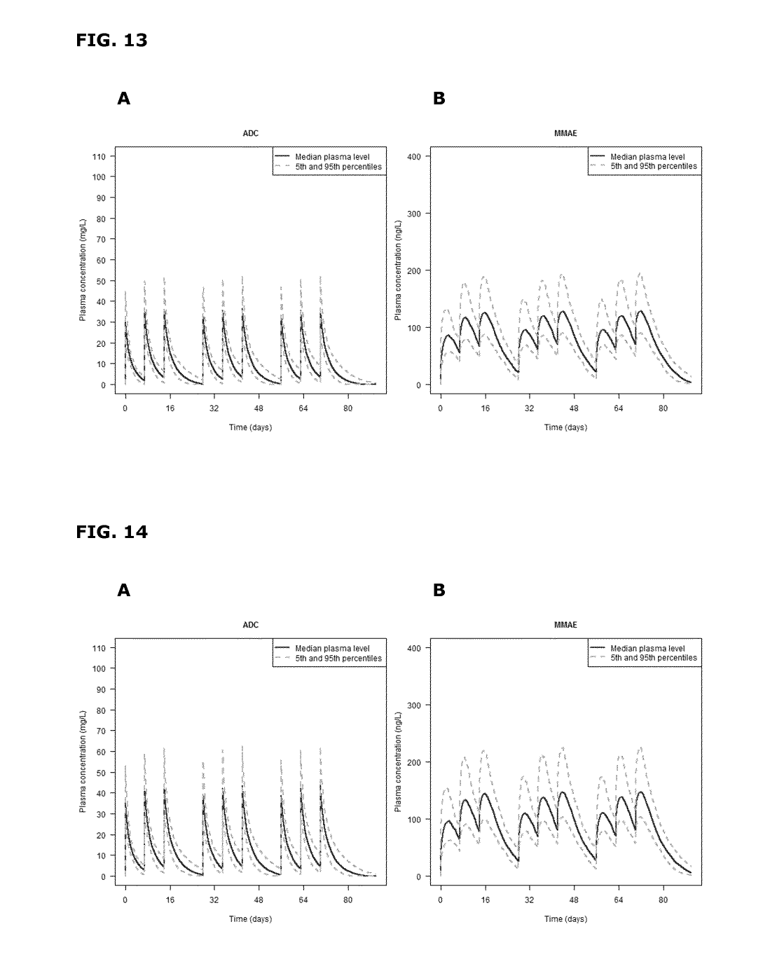

Simulations of the 1Q3W and 3Q4W dosing regimens were done. The results are shown in the tables below and in FIGS. 4 to 14.
1Q3W: Dosing once every 3 weeks. 0.3, 0.6, 1, 1.5, 2, 2.4mg/kg
3Q4W: Weekly dosing for 3 weeks followed by one treatment-free week. 0.6, 0.8, 1, 1.2, 1.4 mg/kg.

TABLE 4
Prediction of ADC exposure parameters in humans, 1Q3W dosing.

| | | | AUC | AUC | AUC | C_{trough} | |
|---|---|---|---|---|---|---|---|
| Dose | | Cₘₐₓ | 1^{st} cycle | 3^{rd} cycle | 12 weeks | 12 weeks | t_{(1/2)} |
| (mg/kg) | Percentile | (mg/L) | (mg * day/L) | (mg * day/L) | (mg * day/L) | (mg/L) | (days) |
| 0.3 | 95% | 9.81 | 11.08 | 11.22 | 44.72 | 0.00 | 1.33 |
| 0.3 | 50% | 6.14 | 6.65 | 6.72 | 26.80 | 0.00 | 0.88 |
| 0.3 | 5% | 3.74 | 2.95 | 2.99 | 11.93 | 0.00 | 0.58 |
| 0.6 | 95% | 21.90 | 33.80 | 34.19 | 136.36 | 0.00 | 1.69 |
| 0.6 | 50% | 14.28 | 24.71 | 24.91 | 99.43 | 0.00 | 1.14 |
| 0.6 | 5% | 9.86 | 14.87 | 14.98 | 59.81 | 0.00 | 0.82 |
| 1 | 95% | 37.83 | 72.67 | 73.07 | 291.87 | 0.00 | 1.91 |
| 1 | 50% | 25.60 | 54.01 | 54.45 | 217.35 | 0.00 | 1.29 |
| 1 | 5% | 17.59 | 36.80 | 37.02 | 147.89 | 0.00 | 0.87 |
| 1.5 | 95% | 57.76 | 124.60 | 126.53 | 502.99 | 0.00 | 2.05 |
| 1.5 | 50% | 39.50 | 94.54 | 95.80 | 382.73 | 0.00 | 1.40 |
| 1.5 | 5% | 27.21 | 66.61 | 67.23 | 268.30 | 0.00 | 0.90 |
| 2 | 95% | 78.15 | 184.23 | 189.80 | 752.76 | 0.48 | 2.19 |
| 2 | 50% | 53.38 | 138.44 | 142.72 | 566.08 | 0.00 | 1.46 |
| 2 | 5% | 36.81 | 96.90 | 98.26 | 391.62 | 0.00 | 0.91 |
| 2.4 | 95% | 94.57 | 233.64 | 245.78 | 967.88 | 0.86 | 2.29 |
| 2.4 | 50% | 64.61 | 175.18 | 179.66 | 713.01 | 0.00 | 1.49 |
| 2.4 | 5% | 44.56 | 121.22 | 122.09 | 487.75 | 0.00 | 0.92 |

TABLE 5
Prediction of MMAE exposure parameters in humans, 1Q3W dosing.

| | | | AUC | AUC | AUC | C_{trough} | | Tₘₐₓ | Tₘₐₓ |
|---|---|---|---|---|---|---|---|---|---|
| | | | 1st cycle | 3rd cycle | 12 weeks | 12 | | 1^{st} | 3^{rd} |
| | | Cₘₐₓ | (ng * day/ | (ng * day/ | (ng * day/ | weeks | t_{(1/2)} | cycle | cycle |
| Dose | Percentile | (ng/L) | L) | L) | L) | (ng/L) | (days) | (days) | (days) |
| 0.3 | 95% | 41.69 | 243.59 | 245.47 | 979.96 | 0.32 | 3.80 | 3.38 | 3.38 |
| 0.3 | 50% | 34.37 | 177.61 | 177.94 | 711.42 | 0.06 | 2.34 | 2.41 | 2.42 |
| 0.3 | 5% | 25.90 | 135.97 | 136.55 | 545.61 | 0.02 | 1.72 | 1.68 | 1.68 |
| 0.6 | 95% | 71.89 | 508.19 | 511.73 | 2043.32 | 1.05 | 6.43 | 3.62 | 3.62 |
| 0.6 | 50% | 53.31 | 380.79 | 381.60 | 1525.57 | 0.22 | 4.25 | 2.78 | 2.78 |
| 0.6 | 5% | 38.67 | 287.54 | 288.20 | 1152.14 | 0.06 | 2.85 | 1.72 | 1.73 |
| 1 | 95% | 114.06 | 841.99 | 852.84 | 3400.38 | 2.67 | 8.05 | 3.74 | 3.74 |
| 1 | 50% | 75.67 | 632.85 | 635.94 | 2540.62 | 0.61 | 5.68 | 2.68 | 2.66 |
| 1 | 5% | 52.29 | 470.70 | 473.11 | 1889.98 | 0.19 | 4.23 | 1.69 | 1.69 |
| 1.5 | 95% | 167.72 | 1244.00 | 1261.79 | 5028.77 | 8.06 | 8.20 | 3.67 | 3.61 |
| 1.5 | 50% | 102.47 | 940.02 | 947.77 | 3781.84 | 1.59 | 6.44 | 2.62 | 2.55 |
| 1.5 | 5% | 69.13 | 675.60 | 696.40 | 2777.89 | 0.55 | 4.26 | 1.67 | 1.67 |
| 2 | 95% | 221.85 | 1678.17 | 1705.56 | 6793.69 | 12.63 | 8.30 | 3.61 | 3.46 |
| 2 | 50% | 128.95 | 1227.71 | 1238.56 | 4943.19 | 3.10 | 6.45 | 2.56 | 2.48 |
| 2 | 5% | 87.82 | 864.30 | 895.42 | 3515.57 | 1.03 | 4.24 | 1.66 | 1.66 |
| 2.4 | 95% | 265.50 | 2021.46 | 2057.77 | 8193.02 | 15.95 | 8.28 | 3.57 | 3.39 |
| 2.4 | 50% | 152.77 | 1449.08 | 1465.60 | 5846.38 | 4.91 | 6.43 | 2.54 | 2.44 |
| 2.4 | 5% | 102.10 | 995.30 | 1046.03 | 4130.28 | 1.61 | 4.23 | 1.66 | 1.66 |

TABLE 6
Prediction of ADC exposure parameters in humans, 3Q4W dosing.

| | | | AUC | AUC | AUC | Ctrough | |
|---|---|---|---|---|---|---|---|
| Dose | | Cmax | 1^{st} cycle | 3^{rd} cycle | 12 weeks | 12 weeks | t_{(1/2)} |
| (mg/kg) | Percentile | (mg/L) | (mg * day/L) | (mg * day/L) | (mg * day/L) | (mg/L) | (days) |
| 0.6 | 95% | 23.60 | 127.75 | 132.41 | 393.05 | 0.06 | 2.05 |
| 0.6 | 50% | 16.00 | 87.77 | 89.20 | 266.35 | 0.00 | 1.31 |
| 0.6 | 5% | 10.73 | 49.33 | 49.52 | 148.37 | 0.00 | 0.88 |
| 0.8 | 95% | 33.12 | 190.78 | 203.60 | 599.63 | 1.02 | 2.41 |
| 0.8 | 50% | 22.91 | 138.05 | 140.84 | 418.89 | 0.00 | 1.53 |
| 0.8 | 5% | 15.66 | 89.31 | 89.90 | 269.10 | 0.00 | 0.93 |
| 1 | 95% | 42.96 | 261.39 | 295.45 | 852.65 | 1.70 | 2.60 |
| 1 | 50% | 29.89 | 194.75 | 201.74 | 600.13 | 0.00 | 1.64 |
| 1 | 5% | 20.53 | 130.58 | 131.74 | 393.72 | 0.00 | 0.98 |
| 1.2 | 95% | 52.82 | 339.78 | 387.07 | 1092.05 | 2.55 | 2.74 |
| 1.2 | 50% | 36.59 | 249.74 | 275.88 | 799.28 | 0.19 | 1.75 |
| 1.2 | 5% | 25.36 | 169.22 | 172.61 | 514.37 | 0.00 | 1.02 |
| 1.4 | 95% | 62.67 | 421.37 | 491.64 | 1390.25 | 3.44 | 2.85 |
| 1.4 | 50% | 43.63 | 313.12 | 343.34 | 986.67 | 0.57 | 1.83 |
| 1.4 | 5% | 30.30 | 209.51 | 215.10 | 639.54 | 0.00 | 1.04 |

TABLE 7
Prediction of MMAE exposure parameters in humans, 3Q4W dosing.

| | | | AUC | AUC | AUC | C_{trough} | | | |
|---|---|---|---|---|---|---|---|---|---|
| | | | 1^{st} cycle | 3^{rd} cycle | 12 weeks | 12 | | Tₘₐₓ 1^{st} | Tₘₐₓ 3^{rd} |
| | | Cₘₐₓ | (ng * day/ | (ng * day/ | (ng * day/ | weeks | t_{(1/2)} | cycle | cycle |
| Dose | Percentile | (ng/L) | L) | L) | L) | (ng/L) | (days) | (days) | (days) |
| 0.6 | 95% | 96.08 | 1576.39 | 1616.66 | 4805.97 | 0.06 | 7.93 | 2.50 | 2.47 |
| 0.6 | 50% | 69.80 | 1183.72 | 1201.06 | 3581.53 | 0.00 | 5.36 | 1.78 | 1.77 |
| 0.6 | 5% | 51.61 | 888.23 | 909.48 | 2699.87 | 0.00 | 3.64 | 1.41 | 1.41 |
| 0.8 | 95% | 129.45 | 2074.30 | 2152.96 | 6332.78 | 1.02 | 8.01 | 2.08 | 2.05 |
| 0.8 | 50% | 90.04 | 1589.02 | 1630.48 | 4860.92 | 0.00 | 6.14 | 1.74 | 1.73 |
| 0.8 | 5% | 63.74 | 1155.05 | 1188.76 | 3535.98 | 0.00 | 4.39 | 1.38 | 1.37 |
| 1 | 95% | 164.01 | 2609.37 | 2711.01 | 8028.25 | 1.70 | 8.11 | 2.06 | 2.03 |
| 1 | 50% | 109.30 | 1964.54 | 2034.37 | 6008.90 | 0.00 | 6.36 | 1.74 | 1.73 |
| 1 | 5% | 76.74 | 1363.82 | 1459.84 | 4224.15 | 0.00 | 4.45 | 1.34 | 1.34 |
| 1.2 | 95% | 195.94 | 3154.94 | 3279.19 | 9708.59 | 2.55 | 8.26 | 2.06 | 2.02 |
| 1.2 | 50% | 128.47 | 2284.50 | 2403.14 | 7076.75 | 0.19 | 6.39 | 1.74 | 1.73 |
| 1.2 | 5% | 90.11 | 1560.00 | 1674.28 | 4919.90 | 0.00 | 4.45 | 1.34 | 1.34 |
| 1.4 | 95% | 226.10 | 3654.89 | 3838.47 | 11347.69 | 3.44 | 8.27 | 2.07 | 2.03 |
| 1.4 | 50% | 146.91 | 2590.28 | 2752.79 | 8087.90 | 0.57 | 6.39 | 1.75 | 1.73 |
| 1.4 | 5% | 103.54 | 1765.00 | 1938.78 | 5615.40 | 0.00 | 4.45 | 1.34 | 1.34 |

### CONCLUSIONS

A two compartment population PK model with linear non-target mediated clearance and non-linear target mediated clearance was generated which is capable of explaining the preclinical PK data obtained in cynomolgus monkeys.

The model was scaled to human in order to predict exposure to HuMax-AXL-ADC and MMAE exposure in human subjects. Different dosing scenarios were simulated predicting that dosing intervals of every three week (1Q3W) lead to negligible accumulation (increase of 1.1% in median AUC between first and third cycle in the 2.4 mg dosing group) of the payload in plasma (see Table 5). However, dosing with a three weeks on one week off schedule (3Q4W) resulted in a slight accumulation of payload in plasma within the cycle. Note that the accumulation of MMAE (AUC cycle 1 vs. AUC cycle 3) was 7.7% in the median percentile for the 1.4 mg/kg dose (Table 7). Interestingly, comparing the AUC of the two different dosing schedules over an equal time interval of 12 weeks with the same cumulative dose (9.6 mg/kg; 2.4 mg/kg 1Q3W vs. 0.8 mg/kg 3Q4W) revealed that the exposure in the more frequent dosing schedule is about 17% below the exposure of the less frequent dosing schedule.

The provided simulations indicate for all proposed dose levels, that MMAE levels would stay well-below 0.3 ng/mL in the 95^{th} percentile. This implies that the planned dosing of HuMax-AXL-ADC is safe with respect to anticipated Cₘₐₓ of MMAE.

### Example 2

### In Vitro Cytotoxicity Induced by MMAE-Conjugated AXL Antibodies

Conjugation of MMAE to Anti-AXL Antibodies

Anti-AXL antibodies were purified by Protein A chromatography according to standard procedures and conjugated to vcMMAE. The drug-linker vcMMAE was alkylated to the cysteines of the reduced antibodies according to procedures described in the literature (see Sun et al., 2005; McDonagh et al., 2006; and Alley et al., 2008). The reaction was quenched by the addition of an excess of N-acetylcysteine. Any residual unconjugated drug was removed by purification and the final anti-AXL antibody drug conjugates were formulated in PBS. The anti-AXL antibody drug conjugates were subsequently analyzed for concentration (by absorbance at 280 nm), the drug to antibody ratio (DAR) by reverse phase chromatography (RP-HPLC) and hydrophobic interaction chromatography (HIC), the amount of unconjugated drug (by reverse phase chromatography), the percentage aggregation (by size-exclusion chromatography, SEC-HPLC) and the endotoxin levels (by LAL). The results are shown below in Table 8.

TABLE 8
Overview of different characteristics of the antibody-drug conjugates.

| | ADC | | | | | | | | | |
|---|---|---|---|---|---|---|---|---|---|---|
| | IgG1- | IgG1- | IgG1- | | | | | | | |
| | AXL- | AXL- | AXL- | IgG1-AXL- | IgG1-AXL- | IgG1-AXL- | IgG1-AXL- | IgG1-AXL- | IgG1-AXL- | IgG1- |
| Assay | 107 | 148 | 154-M103L | 171 | 183-N52Q | 511 | 613 | 726-M101L | 733 | b12 |
| Concentration | 7.18 | 9.63 | 6.57 | 3.69 | 6.71 | 5.77 | 6.17 | 7.37 | 7.71 | 1.58 |
| (mg/mL) | | | | | | | | | | |
| DAR by HIC | 3.97 | 3.96 | 3.71 | 3.65 | 3.92 | 3.87 | 4.23 | 4.12 | 4.08 | 4.00 |
| % | 4.68 | 5.58 | 6.13 | 7.11 | 8.68 | 8.35 | 5.13 | 4.99 | 3.74 | 1.89 |
| unconjugated | | | | | | | | | | |
| antibody | | | | | | | | | | |
| % aggregate | 6.3 | 2.28 | 2.9 | 3.3 | 5.2 | 5.1 | 6.4 | 4.0 | 3.5 | 2.5 |
| by SEC-HPLC | | | | | | | | | | |
| Endotoxin | 2.3 | 1.2 | 2.6 | 3.1 | 5.9 | 4.5 | 2.0 | 3.6 | 7.6 | 11.5 |
| (EU/mg) | | | | | | | | | | |

Cell Culture

LCLC-103H cells (human large cell lung cancer) and A431 cells (DMSZ, Braunschweig, Germany) were cultured in RPMI 1640 with L-Glutamine (Cambrex; cat.no. BE12-115F) supplemented with 10% (vol/vol) heat inactivated Cosmic Calf Serum (Perbio; cat.no. SH30087.03), 2 mM L-glutamine (Cambrex; cat.no. US17-905C), 50 IU/mL penicillin, and 50 pg/mL streptomycin (Cambrex; cat.no. DE17-603E). MDA-MB231 cells were cultured in DMEM with high glucose and HEPES (Lonza #BE12-709F), Donor Bovine Serum with Iron (Life Technologies #10371-029), 2 mM L-glutamine (Lonza #BE17 -605E), 1 mM Sodium Pyruvate (Lonza #BE13-115E), and MEM Non-Essential Amino Acids Solution (Life Technologies #11140). The cell lines were maintained at 37° C. in a 5% (vol/vol) CO₂ humidified incubator. LCLC-103H, A431 and MDA-MB231 cells were cultured to near confluency, after which cells were trypsinized, resuspended in culture medium and passed through a cell strainer (BD Falcon, cat.no. 352340) to obtain a single cell suspension. 1×10³ cells were seeded in each well of a 96-well culture plate, and cells were incubated for 30 min at room temperature and subsequently for 5 hrs at 37⁰C, 5% CO₂ to allow adherence to the plate.

Cytotoxicity Assay

Serial dilutions (final concentrations ranging from 0.00015 to 10 μg/mL) of MMAE-conjugated AXL-antibodies were prepared in culture medium and added to the plates. Incubation of cells with 1 μM staurosporin (#S6942-200, Sigma) was used as reference for 100% tumor cell kill. Untreated cells were used as reference for 100% cell growth. Plates were incubated for 5 days at 37° C., 5% CO₂. Next, CellTiter-Glo Reagent (Promega; cat.no. G7571) was added to the wells (20 μL per well) and plates were incubated for 1.5 hours at 37⁰C, 5% CO₂.

Subsequently, 180 μL per well was transferred to white 96-well Optiplate™ plates (PerkinElmer, Waltham, Mass.; cat.no. 6005299), which were incubated for 30 min at room temperature. Finally, luminescence was measured on an EnVision multiplate reader (Envision, Perkin Elmer).

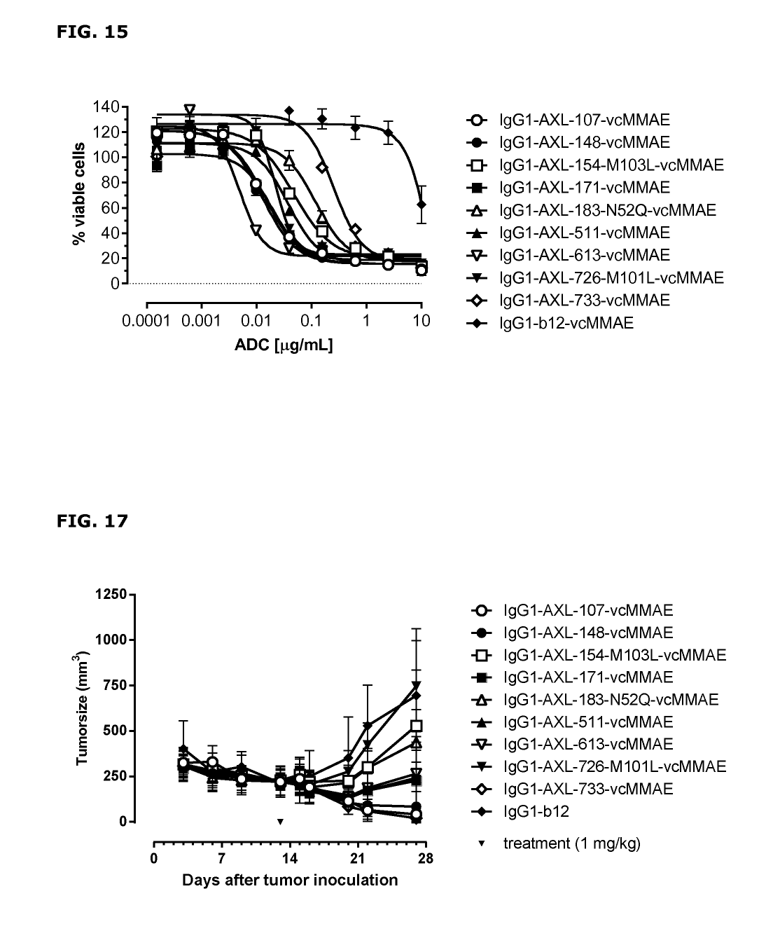

MMAE-conjugated AXL-antibodies induced 50% cell kill in LCLC-103H cells at concentrations between 0.004 and 0.219 μg/mL as shown in Table 9 and FIG. 15.

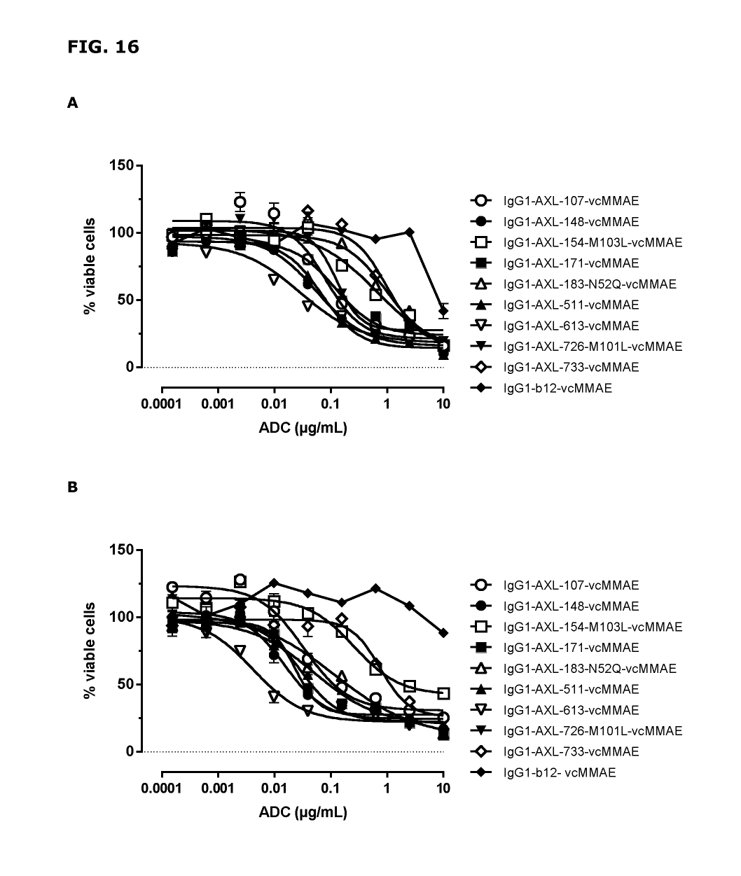

Similarly, AXL-ADCs efficiently induced cytotoxicity in A431 cells (Table 10) and FIG. 16A) and MDA-MB231 cells (Table 10 and FIG. 16B).

TABLE 9
Cytotoxicity of MMAE-conjugated-AXL-antibodies
in LCLC-103H cells (EC50 values)

| ADC | EC50 (μg/mL) |
|---|---|
| IgG1-AXL-613-vcMMAE | 0.004 |
| IgG1-AXL-148-vcMMAE | 0.012 |
| IgG1-AXL-171-vcMMAE | 0.018 |
| IgG1-AXL-726-M101L-vcMMAE | 0.018 |
| IgG1-AXL-107-vcMMAE | 0.022 |
| IgG1-AXL-511-vcMMAE | 0.032 |
| IgG1-AXL-154-M103L-vcMMAE | 0.044 |
| IgG1-AXL-183-N52Q-vcMMAE | 0.113 |
| IgG1-AXL-733-vcMMAE | 0.219 |

TABLE 10
Cytotoxicity of MMAE-conjugated AXL antibodies
in A431 and MDA-MB-231 cells (EC50 values).

| | EC50 (μg/mL) | | | |
|---|---|---|---|---|
| | | | MDA-MB231 | |
| | A431 (n = 3) | | (n = 2) | |
| ADC | Mean | s.d. | Mean | s.d. |
| IgG1-AXL-107-vcMMAE | 0.154 | 0.066 | 0.037 | 0.005 |
| IgG1-AXL-148-vcMMAE | 0.070 | 0.013 | 0.012 | 0.004 |
| IgG1-AXL-154-M103L- | 0.719 | 0.091 | 0.396 | 0.195 |
| vcMMAE | | | | |
| IgG1-AXL-171-vcMMAE | 0.206 | 0.074 | 0.035 | 0.006 |
| IgG1-AXL-183-N52Q-vcMMAE | 1.157 | 0.160 | 0.139 | 0.028 |
| IgG1-AXL-511-vcMMAE | 0.093 | 0.020 | 0.052 | 0.003 |
| IgG1-AXL-613-vcMMAE | 0.109 | 0.078 | 0.005 | 0.001 |
| IgG1-AXL-726-M101L- | 0.270 | 0.157 | 0.022 | 0.002 |
| vcMMAE | | | | |
| IgG1-AXL-733-vcMMAE | 1.253 | 0.228 | 0.881 | 0.182 |

### Example 3

### Therapeutic Treatment of LCLC-103H Tumor Xenografts in SCID Mice with MMAE-Conjugated Anti-AXL Antibodies

The in vivo efficacy of MMAE-conjugated anti-AXL antibodies was determined in established subcutaneous (SC) LCLC-103H xenograft tumors in SCID mice. 5×10⁶ LCLC-103H (large cell lung carcinoma) tumor cells (obtained from Leibniz-Institut DSMZ-Deutsche Sammlung von Mikroorganismen and Zellkulturen GmbH (DSMZ)) in 200 μL PBS were injected subcutaneously in the right flank of female SCID mice. Starting 14-21 days after tumor cell inoculation, when the average tumor size was >100-200 mm³ and distinct tumor growth was observed, a single injection with 1 mg/kg (20 μg/mouse) IgG1-AXL-vcMMAE antibodies or control (unconjugated IgG1-b12) was given intraperitoneally (100 μL/mouse). Tumor volume was determined at least two times per week. Tumor volumes (mm³) were calculated from caliper (PLEXX) measurements as: 0.52×(length)×(width)².

The panel of anti-AXL-vcMMAE antibodies showed a broad range of anti-tumor activity in established SC LCLC-103H tumors (FIG. 17). Clones IgG1-AXL-733-vcMMAE, IgG1-AXL-107-vcMMAE and IgG1-AXL-148-vcMMAE induced tumor regression, whereas clones AXL-171-vcMMAE, IgG1-AXL-511-vcMMAE and IgG1-AXL-613-vcMMAE induced tumor growth inhibition, and clones IgG1-AXL-154-M103L-vcMMAE, IgG1-AXL-183-N52Q-vcMMAE, and IgG1-AXL-726-M101L-vcMMAE (also described in WO 2017/009258) showed no or only minor tumor growth inhibition.

Statistical analysis on the last day that all groups were intact (day 30) using One Way ANOVA (Dunnett's multiple comparisons test versus control IgG1-b12) indicated a highly significant difference (p<0.0001) in tumor volume between IgG1-b12 versus IgG1-AXL-733-vcMMAE, IgG1-AXL-107-vcMMAE and IgG1-AXL-148-vcMMAE. Treatment with these clones led in some mice within these groups to complete tumor reduction. Treatment with clones IgG1-AXL-171-vcMMAE, IgG1-AXL-511-vcMMAE and IgG1-AXL-613-vcMMAE also showed significant tumor growth inhibition compared to IgG1-b12, but the differences were less pronounced (p<0.05 to p<0.001). The tumor growth of mice treated with clones IgG1-AXL-154-M103L-vcMMAE, IgG1-AXL-183-N52Q-vcMMAE, and IgG1-AXL-726-M101L-vcMMAE was not significant affected compared to the IgG1-b12 control.

Anti-tumor activity of anti-AXL-vcMMAE antibodies was observed in various other in vivo tumor models. In two cell line-derived xenograft models (A431; epidermoid adenocarcinoma, and MDA-MB-231; breast cancer) anti-AXL-vcMMAE antibodies induced tumor growth inhibition, and tumor regression was induced by anti-AXL-vcMMAE antibodies in two patient-derived xenograft models from patients with pancreas cancer and cervical cancer.

### Example 4

### Anti-Tumor Efficacy of AXL-ADCs in a Pancreas Cancer Patient-Derived Xenograft (PDX) Model With Heterogeneous Target Expression

The anti-tumor activity of IgG1-AXL-107-vcMMAE, IgG1-AXL-148-vcMMAE, and IgG1-AXL-733-vcMMAE was determined in the PAXF1657 pancreas cancer PDX model (experiments performed by Oncotest, Freiburg, Germany). Human pancreas tumor tissue was subcutaneously implanted in the left flank of 5-7 weeks old female NMRI nu/nu mice. Randomization of animals was performed as follows: animals bearing a tumor with a volume between 50-250 mm³, preferably 80-200 mm³, were distributed in 7 experimental groups (8 animals per group), considering a comparable median and mean of group tumor volume. At day of randomization (day 0), the 3 ADCs were dosed intravenously (i.v.) at either 4 mg/kg or 2 mg/kg, and the control group received a single dose of IgG1-b12 (4 mg/kg). Tumor volumes (mm³) were monitored twice weekly and were calculated from caliper (PLEXX) measurements as: 0.52×(length)×(width)².

Staining of PAXF1657 tumors was performed with standard immunohistochemistry techniques. Briefly, frozen tissues were fixated with acetone for 10 minutes and endogenous peroxidase was exhausted using hydrogen peroxidase. Subsequently, tissue sections were blocked with normal mouse serum and staining was performed by incubation with 5 μg/mL of a pool of 5 IgG1-AXL antibodies (IgG1-AXL-061, IgG1-AXL-137, IgG1-AXL-148, IgG1-AXL-183, IgG1-AXL-726). After incubation with the secondary, horseradish peroxidase (HRP) conjugated antibody, HRP was visualized with amino-ethyl carbazole (AEC; resulting in a red color). Each slide was counterstained with hematoxylin (blue) to identify nuclei and coverslipped in glycergel. Immunostained tissue slices were digitized on manual Zeiss microscope (AxioSkop) at 10× and 40× magnifications. The results showed heterogeneous AXL expression in PAXF1657 tumors. Whereas strong AXL staining is observed in some tumor cells, other cells do not show AXL staining.

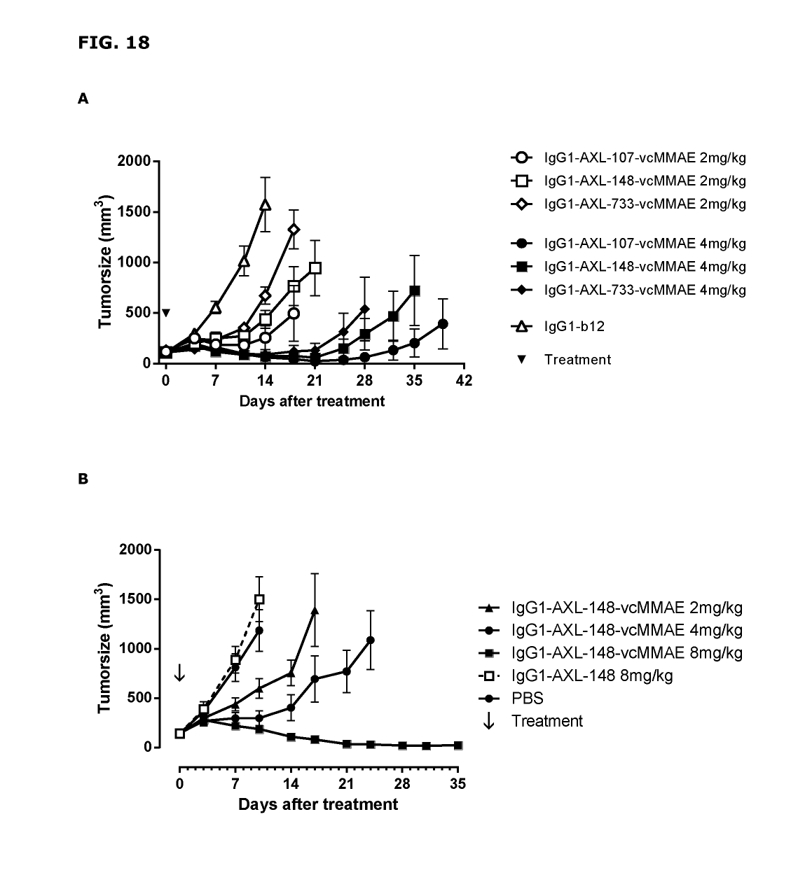

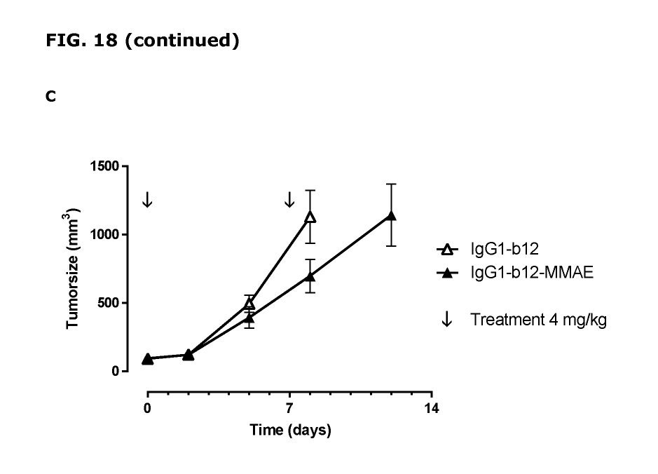

FIG. 18A shows that treatment of mice with 2 mg/kg IgG1-AXL-107-vcMMAE, IgG1-AXL-148-vcMMAE and IgG1-AXL-733-vcMMAE significantly reduced the growth of PAXF1657 tumors compared to the control group. At a dose of 4 mg/kg IgG1-AXL-107-vcMMAE, IgG1-AXL-148-vcMMAE and IgG1-AXL-733-vcMMAE induced tumor regression of PAXF1657 tumors. On day 14 after treatment, the average tumor size in mice that had been treated with 2 mg/kg or 4 mg/kg IgG1-AXL-107-MMAE, IgG1-AXL-148-MMAE or IgG1-AXL-733-MMAE was significantly smaller than in mice that had been treated with an isotype control IgG (IgG1-b12) (p<0.001; Tukey's multiple comparison test).

Treatment of mice with unconjugated IgG1-AXL-148 did not result in anti-tumor activity in the PAXF1657 model (FIG. 18B). Conjugated IgG1-AXL-148-vcMMAE, however, induced dose-dependent antitumor activity in this model (FIG. 18B), illustrating that the therapeutic capacity of AXL-ADCs is dependent on the cytotoxic activity of MMAE.

Moreover, treatment of mice with the untargeted ADC IgG1-b12-vcMMAE did not show anti-tumor activity in the PAXF1657 model (FIG. 18C), illustrating that the therapeutic capacity of AXL-ADCs also depends on specific target binding.

### Example 5

### In Vivo Anti-Tumor Efficacy of AXL-ADCs in Xenograft Models With and Without Autocrine (Endogenous) Gas6 Production

Gas6 Production of A431 and LCLC-103H Tumor Cells

It was tested whether A431 cells and LCLC-103H cells produce Gas6. Therefore, cells were grown in complete culture medium for 3 days. Gas6 levels in supernatant were determined using the Quantikine Human Gas6 ELISA (R&D Systems, Minneapolis, Minn.) according to manufacturer's instructions. This assay uses the quantitative sandwich ELISA technique. A monoclonal Ab specific for human Gas6 has been pre-coated onto a microplate. Standards and samples are pipetted into the wells and any human Gas6 present is bound by the immobilized Ab. After washing away any unbound substances, an enzyme-linked polyclonal Ab specific for human Gas6 is added to the wells. Following a wash to remove any unbound Ab-enzyme reagent, a substrate is added to the wells and color develops in proportion to the amount of human Gas6 bound in the initial step. The color development is stopped and the intensity of the color is measured.

Cell culture medium conditioned by A431 cells was found to contain 2576 ng/mL Gas6, while the concentration of Gas6 in medium conditioned by LCLC-103H cells was more than 20-fold less (Table 11).

TABLE 11
Gas6 production in tumor cell conditioned medium.

| Cell line | Gas6 in supernatant (ng/mL) |
|---|---|
| LCLC-103H | 126 |
| A431 | 2576 |

Anti-Tumor Activity of AXL-ADCs In Vivo

The in vivo anti-tumor activity of several AXL-ADCs—IgG1-AXL-061-vcMMAE (Ig1 binder), IgG1-AXL-107-vcMMAE (Ig1-binder), IgG1-AXL-137-vcMMAE (Ig1-binder), IgG1-AXL-148-vcMMAE (Ig2-binder), IgG1-AXL-183-vcMMAE (FN1-binder), and IgG1-AXL-726-vcMMAE (FN2-binder)—was determined in the A431 (epidermoid carcinoma) tumor model, that produces high levels of Gas6, and the LCLC-103H (large cell lung carcinoma) tumor model, that produces low levels of Gas6. IgG1-AXL-107 does not compete with Gas6 binding, whereas IgG1-AXL-061 and IgG1-AXL-137 compete with Gas6 for binding to AXL.

Tumor induction was performed by subcutaneous injection of 5×10⁶ A431 or LCLC-103H tumor cells (both obtained from Leibniz-Institut—Deutsche Sammlung von Mikroorganismen and Zellkulturen GmbH (DSMZ)) in 200 μL PBS in the right flank of female SCID mice. Treatment was started 14-21 days after tumor cell inoculation, when the average tumor size was >100-200 mm³ and distinct tumor growth was observed. Mice received a single injection or a total of 4 biweekly intraperitoneal injections with IgG1-AXL-vcMMAE ADCs or control antibody (unconjugated IgG1-b12), as indicated. Tumor volume was determined at least two times per week. Tumor volumes (mm³) were calculated from caliper (PLEXX) measurements as: 0.52×(length)×(width)².

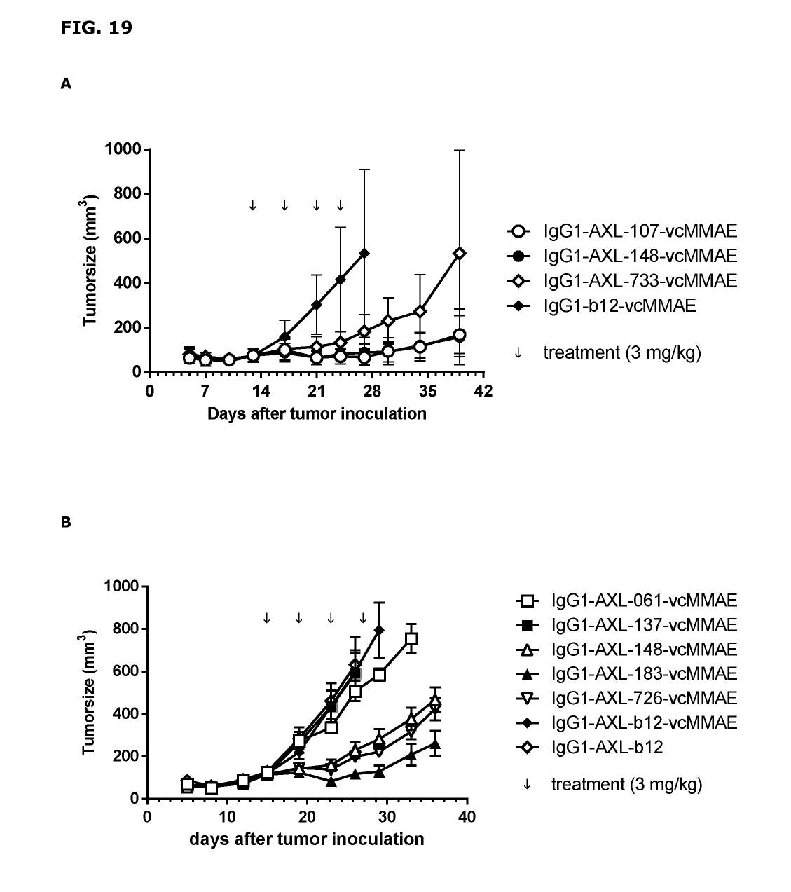

FIG. 19A shows that treatment of mice with 3 mg/kg IgG1-AXL-107-vcMMAE, IgG1-AXL-148-vcMMAE and IgG1-AXL-733-vcMMAE induced growth inhibition of A431 tumors.

FIG. 19B shows that treatment of mice with 3 mg/kg IgG1-AXL-148-vcMMAE, IgG1-AXL-183-vcMMAE (FN1 binder) and IgG1-AXL-726-vcMMAE (FN2 binder) induced growth inhibition of A431 tumors. In contrast, clones IgG1-AXL-061-vcMMAE and IgG1-AXL-137-vcMMAE did not show anti-tumor activity in the A431 xenograft model.

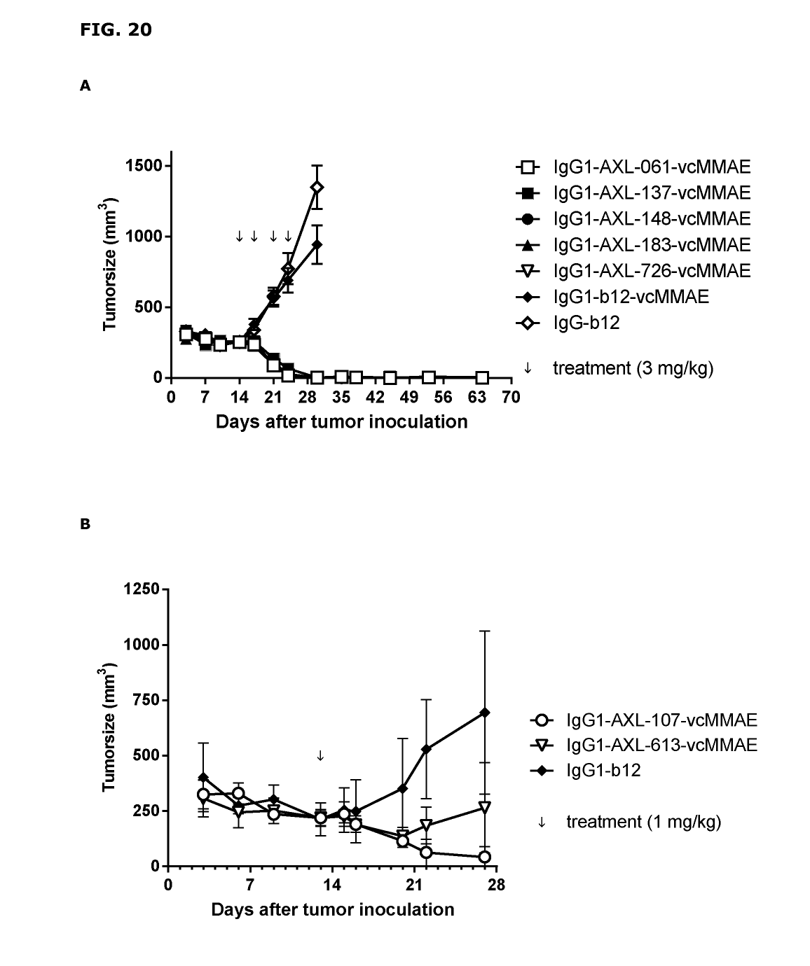

FIG. 20A shows that treatment of mice with 3 mg/kg IgG1-AXL-061-vcMMAE, IgG1-AXL-137-vcMMAE, IgG1-AXL-148-vcMMAE, IgG1-AXL-183-vcMMAE and IgG1-AXL-726-vcMMAE induced tumor regression in the LCLC-103H xenograft model. Similarly, treatment of mice with 1 mg/kg IgG1-AXL-107-vcMMAE or 1 mg/kg IgG1-AXL-613-vcMMAE induced regression of LCLC-103H tumors (FIG. 20B).

In summary, all AXL-ADCs showed anti-tumor activity in the LCLC-103H xenograft model that produces low levels of Gas6. In the A431 xenograft model, that produces high levels of Gas6, anti-tumor activity was only observed for those AXL-ADCs that did not compete with the AXL ligand Gas6.

### Example 6

### AXL Expression in Different Tumor Indications

Expression of AXL was evaluated in freshly cut paraffin embedded and formalin fixated (FFPE) tumor tissue micro arrays (TMA) comprising tissue cores from patients with thyroid, esophageal, ovarian, pancreatic, lung, breast, cervical or endometrial cancer, or malignant melanoma. TMAs were obtained from US BioMax.

FFPE tumor array slides were deparaffinized and subjected to antigen retrieval (pH 6) and endogenous peroxidase was exhausted by incubation with 0.1% H2O2 in citrate/phosphate buffer. To detect AXL expression, the TMAs were incubated with rabbit-anti-AXL (Santa Cruz, cat nr: sc-20741) at a concentration of 1 μg/mL for 60 min (room temperature (RT)). To identify (tumor) cells of epithelial origin, TMAs were incubated with rabbit-anti-cytokeratin (Abcam, cat. Nr. ab9377) at a dilution of 1:50 for 60 min (RT). After a washing step, the IMAs were incubated with peroxidase conjugated, anti-rabbit IgG dextran polymer (ImmunoLogic, cat no: DPVR55HRP) to detect binding of rabbit Anti-AXL and rabbit anti-cytokeratin antibodies. Finally, binding of anti-rabbit IgG dextran polymer was visualized with di-amino-benzadine (DAB; brown color; DAKO, cat no: K346811). In the TMA with malignant melanoma tissue cores, binding of anti-rabbit IgG dextran polymer was visualized with amino-ethyl carbazole (AEC; red color; Vector, SK4200). Nuclei in IMAs were visualized with hematoxylin (blue color).

AXL and cytokeratin immunostained IMAs were digitized with an Aperio slide scanner at 20× magnification and immunostaining was quantified with tissue image analysis software (Definiens Tissue Studio software, version 3.6.1), using a cell-based algorithm. The algorithm was designed to identify and quantify the percentage of AXL- or cytokeratin-positive cells in the biopsies (range 0-100%) and to quantify AXL staining intensity in AXL-positive tumor cells (optical density (OD); range 0-3) in each tumor core. Tumor cells were scored AXL positive, when AXL OD was at least 0.1. The percentage of AXL positive tumor cells per tumor core (range 0-100%) was calculated by dividing the total number of AXL positive cells by the total number of cytokeratin-positive cells in sequential tumor cores. The average AXL staining intensity (OD) in each tumor core was calculated by dividing the sum of AXL OD of all AXL positive tumor cells by the number of AXL positive tumor cells.

Tumor array from patients with malignant melanoma were scored manually. AXL staining intensity was scored as either weak (1+), moderate (2+) or strong (3+) and the percentage AXL positive melanoma cells was scored in 10% intervals (range 0-100%).

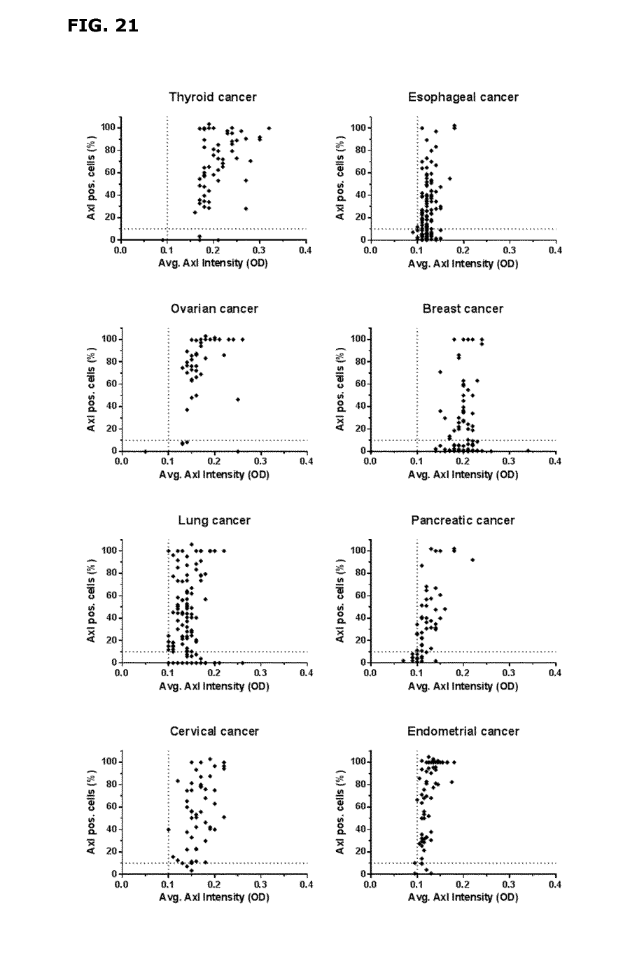

FIG. 21 provides a graphical representation of AXL expression in tumor cores of thyroid, esophageal, ovarian, breast, lung, pancreatic, cervical and endometrial cancer. Table 12 shows the percentage of tumor cores that showed AXL expression in more than 10% of tumor cells, for each indication. Tissue cores immunostained for AXL illustrated heterogeneous expression of AXL in the tumor issue.

TABLE 12

| | | % tumor cores |
|---|---|---|
| | | (patients) with |
| | | >10% AXL-positive |
| Tumor indication | Subtype | tumor cells |
| Esophageal cancer | Adenocarcinoma (n = 19) | 73 |
| | Squamous cell carcinoma | 55 |
| | (n = 60) | |
| Ovarian cancer | All subtypes (n = 52) | 90 |
| Pancreatic cancer | All subtypes (n = 58) | 60 |
| Lung cancer (NSCLC) | Squamous cell carcinoma | 63 |
| | SSC (n = 52) | |
| | Adenocarcinoma (n = 48) | 67 |
| Lung cancer (SCLC) | SCLC (n = 5) | 60 |
| Thyroid cancer | All subtypes (n = 48) | 92 |
| Uterine cancer | All subtypes (n = 60) | 88 |
| Breast cancer | TNBC (n = 54) | 24 |
| Cervical cancer | All subtypes (n = 54) | 93 |
| Melanoma | Malignant melanoma | 6 |
| | (n = 67) | |

Abbreviations used:
NSCLC, non small cell lung cancer;
SLCL, small cell lung cancer;
TNBC, triple negative breast cancer

### Example 7

### Anti-Tumor Efficacy of AXL-ADCs in an Esophageal Cancer Patient-Derived Xenograft (PDX) Model

The anti-tumor activity of IgG1-AXL-107-vcMMAE (also referred to as “HuMax-AXL-ADC” herein) was evaluated in the subcutaneous esophageal PDX model ES0195 in BALB/c nude mice (experiments performed by Crown Bioscience. Taicang Jiangsu Province, China). Tumor fragments from donor mice bearing patient-derived esophageal xenografts (ES0195) were used for inoculation into BALB/c nude mice. Each mouse was inoculated subcutaneously at the right flank with one tumor fragment (2-3 mm in diameter) and tumors were allowed to grow until the tumor volume was about 150 mm³. Randomization of animals was performed as follows: animals bearing a tumor with a volume of about 150 mm³ were distributed in 5 experimental groups (8 animals per group), considering a comparable median and mean of group tumor volume. The treatment groups were: IgG1-b12, IgG1-b12-vcMMAE, IgG1-AXL-107, IgG1-AXL-107-vcMMAE, and paclitaxel. The antibodies and ADCs were dosed intravenously (i.v.) at 4 mg/kg at day of randomization (day 0) and day 7. Paclitaxel was dosed intra-peritoneally (i.p.) at 20 mg/kg at day 0, 7, and 14. Tumor volumes (mm³) were monitored twice weekly and were calculated from caliper (PLEXX) measurements as: 0.52×(length)×(width)².

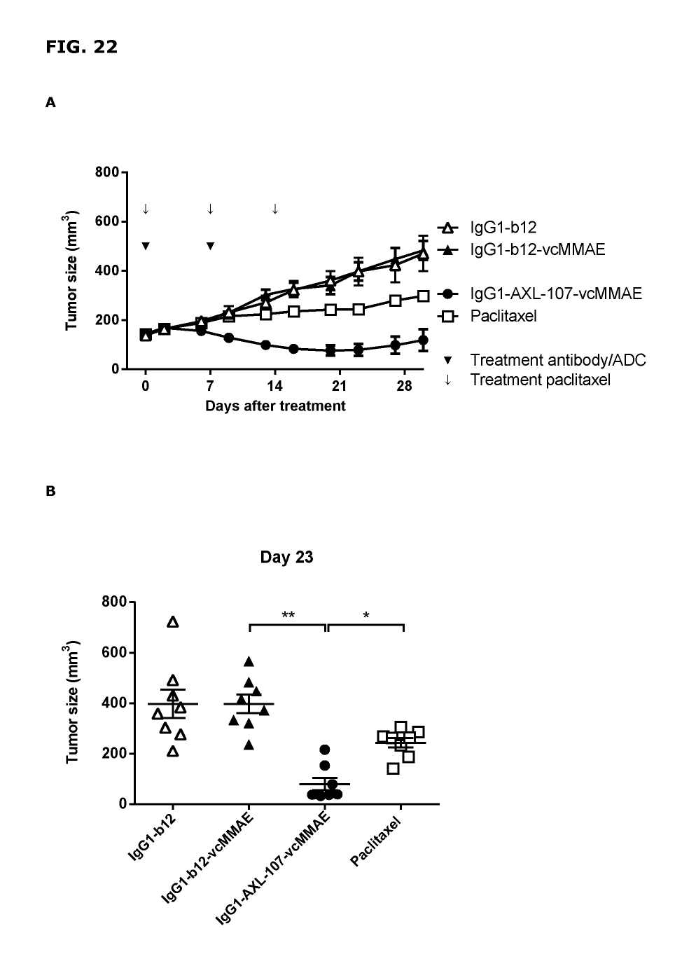

FIG. 22 shows that treatment of mice with IgG1-AXL-107-vcMMAE induced tumor regression of ES0195 tumors compared to the IgG1-b12 and IgG1-b12-MMAE control groups (p<0.001 at day 23, one-way ANOVA test). Treatment of mice with the untargeted ADC IgG1-b12-vcMMAE did not show anti-tumor activity in this model, illustrating that the therapeutic capacity of AXL-ADCs depends on specific target binding. Mice that were treated with paclitaxel showed tumor growth inhibition, but this was less effective compared to treatment with IgG1-AXL-107-vcMMAE (p<0.05 at day 23, one-way ANOVA test).

### Example 8

### Anti-Tumor Efficacy of AXL-ADC in a Cervical Cancer Patient-Derived Xenograft (PDX) Model

The anti-tumor activity of IgG1-AXL-183-vcMMAE and IgG1-AXL-726-vcMMAE was evaluated in the patient derived cervix carcinoma xenograft CEXF 773 model in NMRI nu/nu mice (Harlan, Netherlands). Experiments were performed by Oncotest (Freiburg, Germany).

Tumor fragments were obtained from xenografts in serial passage in nude mice. After removal from donor mice, tumors were cut into fragments (4-5 mm diameter) and placed in PBS (with 10% penicillin/streptomycin) until subcutaneous implantation. Mice under isofluorane anesthesia received unilateral, subcutaneous tumor implants in the flank. Tumors were allowed to grow until the tumor volume was 50-250 mm³.

Randomization of animals was performed as follows: animals bearing a tumor with a volume of 50-250 mm³ were distributed in 4 experimental groups (8 animals per group), considering a comparable median and mean of group tumor volume. The treatment groups were: IgG1-b12, IgG1-b12-vcMMAE, IgG1-AXL-183-vcMMAE and IgG1-AXL-726-vcMMAE. The antibodies and ADCs were dosed intravenously (i.v.) at 4 mg/kg on the day of randomization (day 0) and on day 7. Tumor volumes (mm³) were monitored twice weekly and were calculated from caliper (PLEXX) measurements as: 0.52×(length)×(width)².

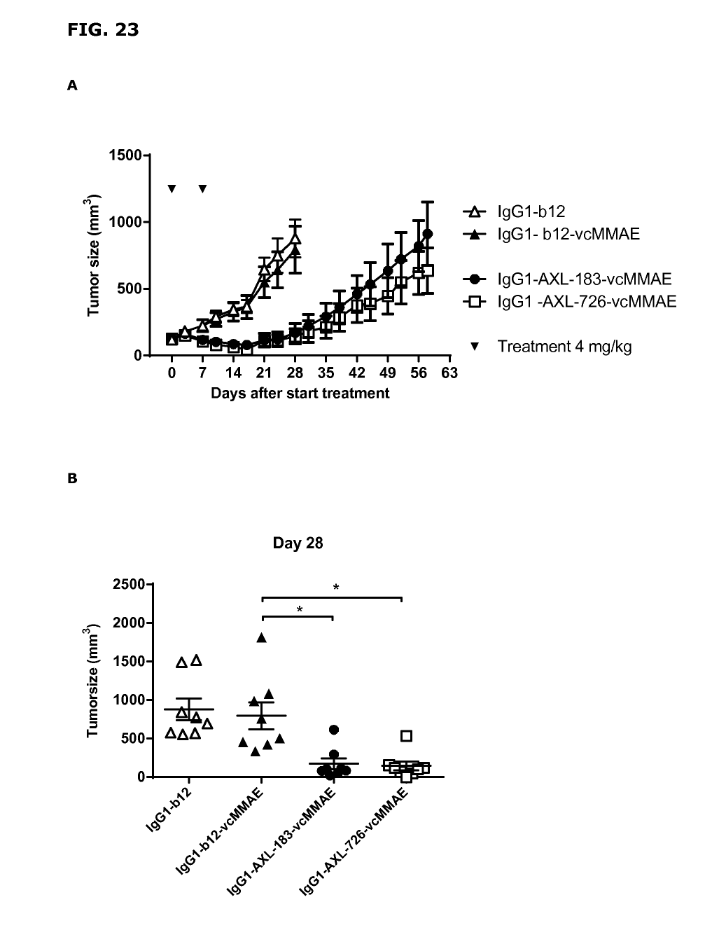

FIG. 23 shows that treatment of mice with IgG1-AXL-183-vcMMAE or IgG1-AXL-726-vcMMAE induced tumor regression of CEXF 773 tumors compared to the IgG1-b12 and IgG1-b12-MMAE control groups. Treatment of mice with the untargeted ADC IgG1-b12-vcMMAE did not show anti-tumor activity in this model, illustrating that the therapeutic capacity of AXL-ADCs depends on specific target binding. Statistical analysis of tumor size at day 28 (Kruskal-Wallis and Mantel-Cox using a tumor size cut-off 500 mm³), showed that the average tumor size in mice treated with IgG1-AXL-183-vcMMAE or IgG1-AXL-726-vcMMAE was significantly smaller than in mice that had been treated with IgG1-b12 and IgG1-b12-vcMMAE (p<0.001). IgG1-AXL-183-vcMMAE and IgG1-AXL-726-vcMMAE were equally effective.

### Example 9

### Anti-Tumor Efficacy of AXL-ADCs in an Orthotopic Breast Cancer Xenograft Model

The anti-tumor activity of IgG1-AXL-183-vcMMAE and IgG1-AXL-726-vcMMAE was evaluated in in an orthotopic MDA-MB-231 D3H2LN xenograft model.

MDA-MB-231-luc D3H2LN Bioware cells (mammary gland adenocarcinoma; Perkin Elmer, Waltham, Mass.) were implanted in the mammary fat pad of 6-11 week old, female SCID (C.B-17/IcrPrkdc-scid/CRL) mice (Charles-River) under isofluorane anesthesia. Tumors were allowed to grow and mice were randomized when tumors reached a volume of ˜325 mm³. Therefore, mice were distributed in 4 experimental groups (6-7 animals per group), considering a comparable median and mean of group tumor volume. The treatment groups were: IgG1-b12, IgG1-b12-vcMMAE, IgG1-AXL-183-vcMMAE and IgG1-AXL-726-vcMMAE. The animals received a total of 4 biweekly doses of 3 mg/kg antibody or ADC starting at the day of randomization. Tumor volumes (mm³) were monitored twice weekly and were calculated from caliper (PLEXX) measurements as: 0.52×(length)×(width)².

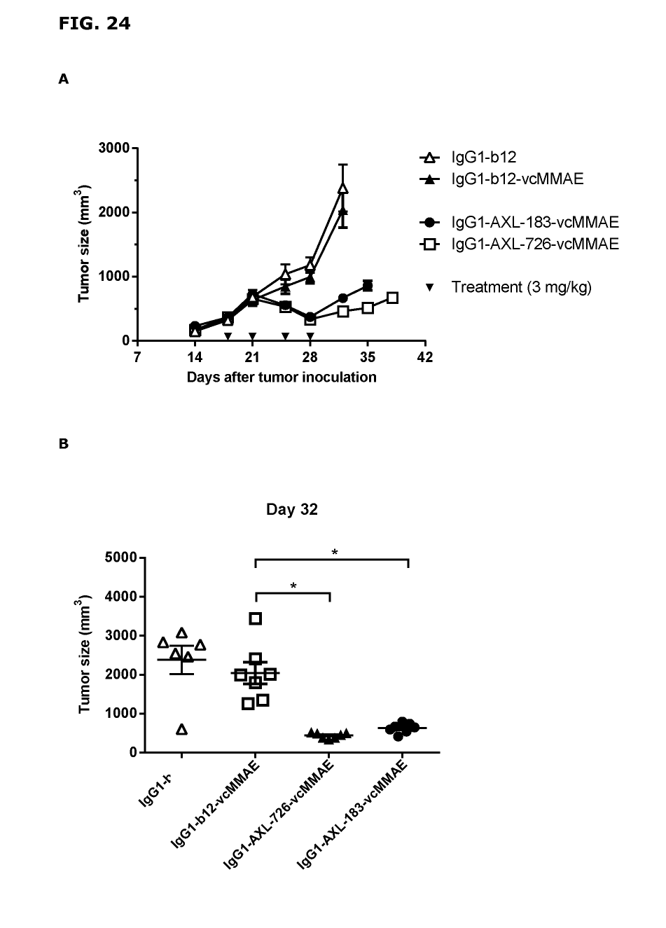

FIG. 24 shows that treatment of mice with IgG1-AXL-183-vcMMAE or IgG1-AXL-726-vcMMAE induced tumor regression of MDA-MB-231 tumors compared to the IgG1-b12 and IgG1-b12-MMAE control groups. Treatment of mice with the untargeted ADC IgG1-b12-vcMMAE did not show anti-tumor activity in this model, showing that the therapeutic capacity of AXL-ADCs depends on specific target binding. Statistical analysis of tumor size at day 32 (One Way Anova test), showed that the average tumor size in mice that had been treated with IgG1-AXL-183-vcMMAE or IgG1-AXL-726-vcMMAE was significantly smaller than in mice that had been treated with IgG1-b12 and IgG1-b12-vcMMAE (P<0.001). No differences were observed between the IgG1-AXL-183-vcMMAE and IgG1-AXL-726-vcMMAE treatment groups, illustrating that these induced equally effective anti-tumor activity.

### Example 10

### Improved Anti-Tumor Efficacy of IgG1-AXL-107-vcMMAE in Combination with Erlotinib in a NSCLC Patient-Derived Xenograft (PDX) Model

LU2511 PDX Model

The anti-tumor activity of IgG1-AXL-107-vcMMAE was evaluated in the subcutaneous erlotinib-resistant NSCLC PDX model LU2511 in BALB/c nude mice (experiments performed by Crown Bioscience, Changping District, Beijing, China). Tumor fragments from donor mice bearing patient-derived NSCLC xenografts (LU2511) were used for inoculation into BALB/c nude mice. Each mouse was inoculated subcutaneously at the right flank with one tumor fragment (2-3 mm in diameter) and tumors were allowed to grow until the tumor volume was about 200 mm³. Randomization of animals was performed as follows: animals bearing a tumor with a volume of about 200 mm³ were distributed in 5 experimental groups (8 animals per group), considering a comparable median and mean of group tumor volume. The treatment groups were: IgG1-b12, IgG1-b12-vcMMAE, IgG1-AXL-107-vcMMAE, erlotinib, and erlotinib plus IgG1-AXL-107-vcMMAE. The antibodies and ADCs were dosed intravenously (i.v.) at 4 mg/kg on the day of randomization (day 0) and on day 7. Erlotinib was dosed orally (per os) at 50 mg/kg daily for 2 weeks. Tumor volumes (mm³) were monitored twice weekly and were calculated from caliper (PLEXX) measurements as: 0.5×(length)×(width)².

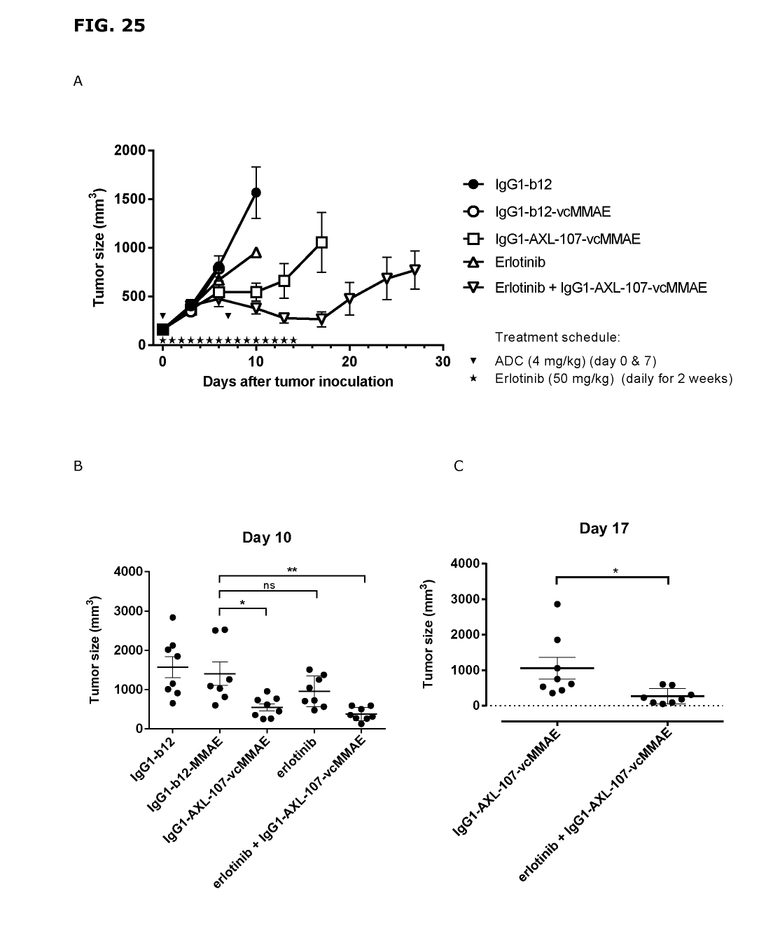

FIG. 25 shows that treatment of mice with erlotinib did not induce anti-tumor activity, which was expected. IgG1-AXL-107-vcMMAE induced tumor growth inhibition of LU2511 tumors compared to the IgG1-b12 (p<0.01 at day 10, one-way ANOVA test; FIG. 25B) and IgG1-b12-MMAE (p<0.05 at day 10, one-way ANOVA test; FIG. 25B) control groups. Treatment of mice with the combination of IgG1-AXL-107-vcMMAE and erlotinib induced more potent anti-tumor activity than IgG1-AXL-107-vcMMAE alone in this model (p<0.05 at day 17, one-way ANOVA test; FIG. 25C).

LU0858 PDX Model

The anti-tumor activity of IgG1-AXL-107-vcMMAE was evaluated in the subcutaneous erlotinib-resistant NSCLC PDX model LU0858 in BALB/c nude mice (experiments performed by CrownBioscience, Changping District, Beijing, China). Inoculation of tumor fragments into BALB/c nude mice and randomization was performed as described above.

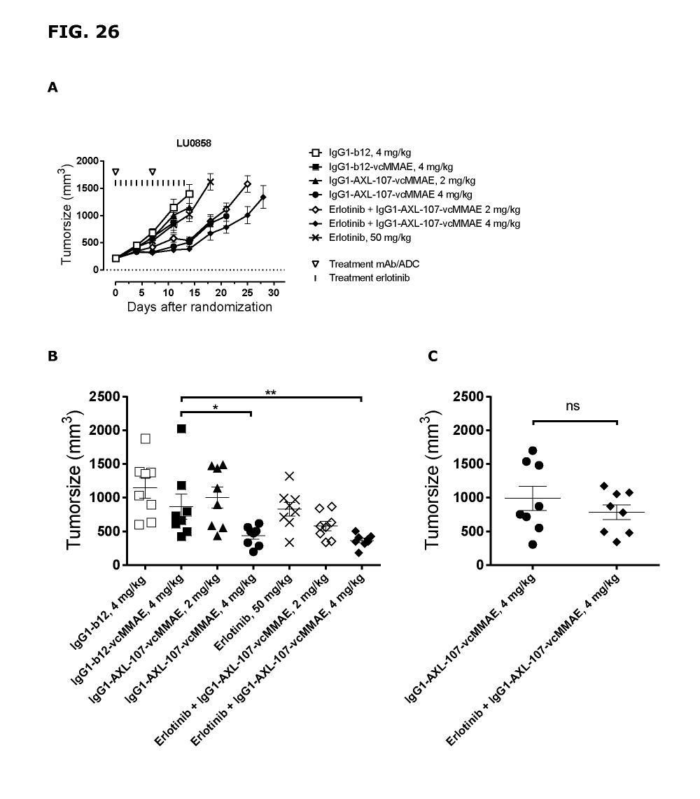

Treatment with IgG1-AXL-107-vcMMAE (2 or 4 mg/kg) was performed at day 0 and 7 after randomization of the groups (FIG. 26). IgG1-AXL-107-vcMMAE treatment in combination with EGFR inhibitor erlotinib was also tested. Erlotinib was given daily for 14 days at a dose of 50 mg/kg. Erlotinib alone, IgG1-b12-vcMMAE and IgG1-b12 were used as controls. Erlotinib alone had no effect on tumor growth. At 2 mg/kg, IgG1-AXL-107-vcMMAE alone had no effect on tumor growth. At 4 mg/kg, IgG1-AXL-107-vcMMAE alone induced tumor growth inhibition compared to the IgG1-b12-vcMMAE control. The combination of 4 mg/kg IgG1-AXL-107-vcMMAE with erlotinib did not improve the outcome versus IgG1-AXL-107-vcMMAE alone (FIG. 26). Addition of erlotinib to the 2 mg/kg IgG1-AXL-107-vcMMAE treatment led to similar growth inhibition as the group that received 4 mg/kg IgG1-AXL-107-vcMMAE.

LU1868 PDX Model

The anti-tumor activity of IgG1-AXL-107-vcMMAE was evaluated in the subcutaneous erlotinib-resistant NSCLC PDX model LU1858 in BALB/c nude mice (experiments performed by CrownBioscience, Changping District, Beijing, China). Inoculation of tumor fragments into BALB/c nude mice and randomization was performed as described above.

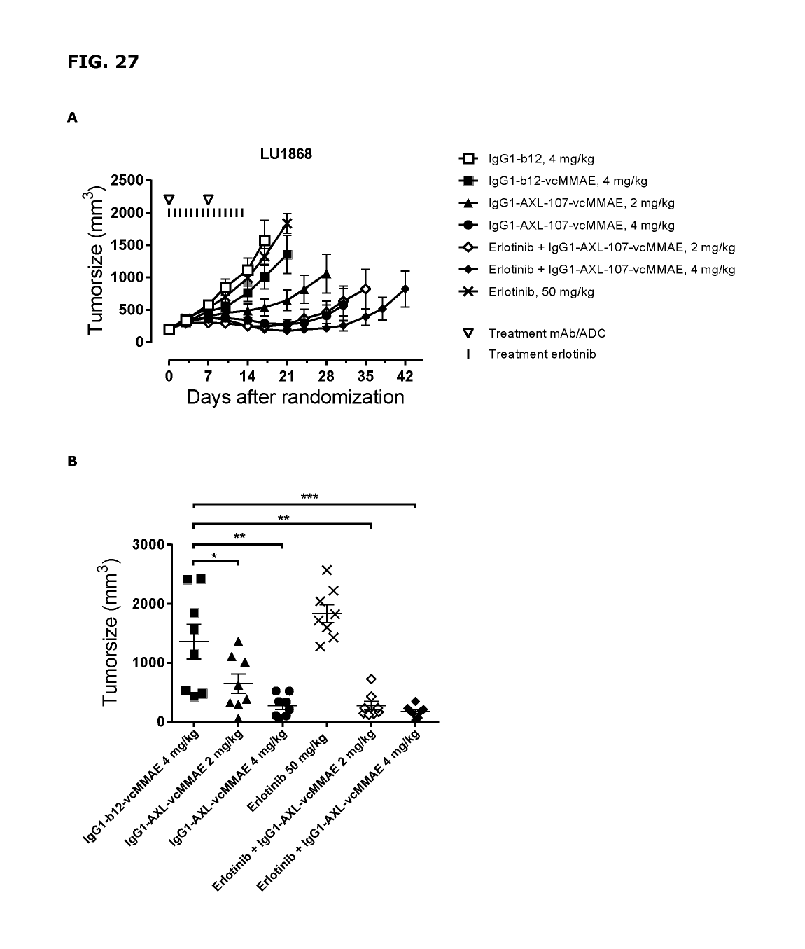

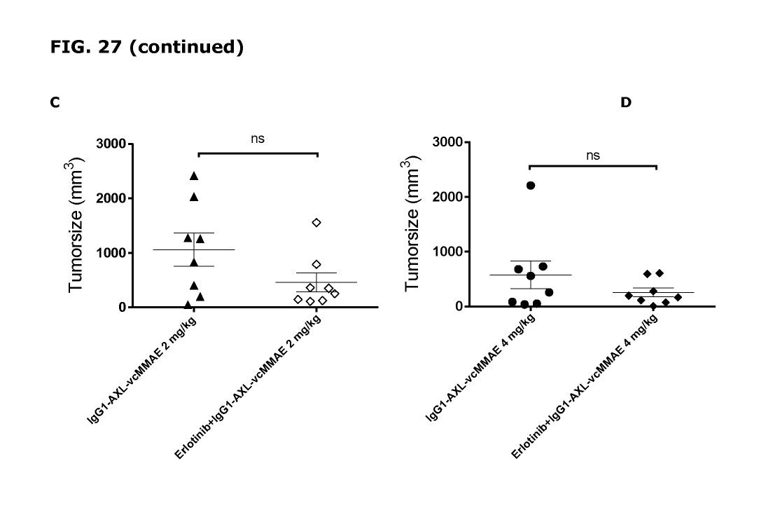

Treatment with IgG1-AXL-107-vcMMAE (2 or 4 mg/kg) was performed at day 0 and 7 after randomization of the groups. IgG1-AXL-107-vcMMAE treatment in combination with EGFR inhibitor erlotinib was also tested. Erlotinib was given daily for 14 days at a dose of 50 mg/kg. Treatments with erlotinib alone, IgG1-b12-vcMMAE or IgG1-b12 were included as controls (FIG. 27).

Analysis by Mann-Whitney test was done on day 21 to compare treatment effects versus IgG1-b12 or IgG1-b12-vcMMAE, on day 28 to compare the effects of IgG1-AXL-107-vcMMAE 2 mg/kg alone versus IgG1-AXL-107-vcMMAE 2 mg/kg in combination with erlotinib, and on day 31 to compare the effects of IgG1-AXL-107-vcMMAE 4 mg/kg alone versus IgG1-AXL-107-vcMMAE 4 mg/kg in combination with erlotinib. Erlotinib alone had no effect on tumor growth. At 2 mg/kg and 4 mg/kg, IgG1-AXL-107-vcMMAE alone induced tumor growth inhibition, while the combination of IgG1-AXL-107-vcMMAE with erlotinib did not improve the outcome versus IgG1-AXL-107-vcMMAE alone (FIG. 27).

LXFA 526 PDX Model

The anti-tumor activity of IgG1-AXL-107-vcMMAE was evaluated in the subcutaneous erlotinib-resistant NSCLC PDX model LXFA 526 (experiments performed by Oncotest, Freiburg, Germany). Inoculation of tumor fragments into 4-6 weeks old male NMRI nu/nu mice and randomization was performed as described above.

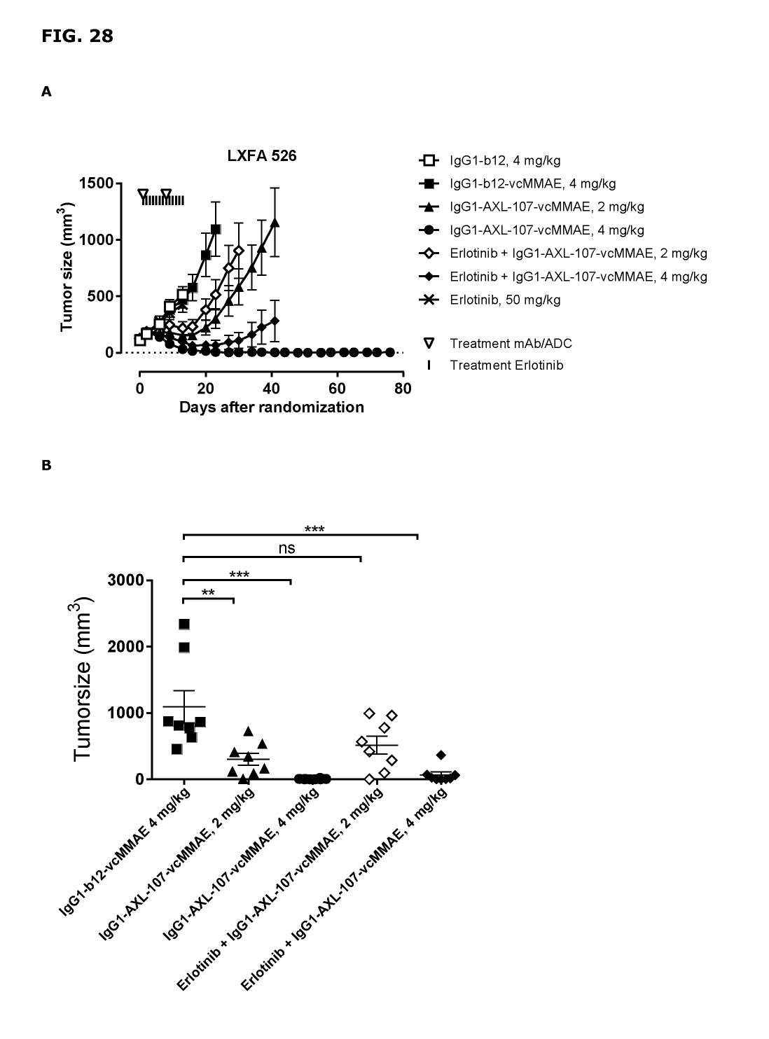

Treatment with IgG1-AXL-107-vcMMAE (2 or 4 mg/kg) was performed at day 0 and 7 after randomization of the groups (FIG. 28). IgG1-AXL-107-vcMMAE treatment in combination with EGFR inhibitor erlotinib was also tested. Erlotinib was given daily for 14 days at a dose of 50 mg/kg. Erlotinib alone, IgG1-b12-vcMMAE and IgG1-b12 were used as control. Erlotinib alone had no effect on tumor growth. IgG1-AXL-107-vcMMAE induced tumor growth inhibition at a dose of 2 mg//kg, while at a dose of 4 mg/kg, IgG1-AXL-107-vcMMAE induced complete tumor regression in all mice at least until day 76. Combination treatment of IgG1-AXL-107-vcMMAE at dose levels of 2 mg/kg or 4 mg/kg with erlotinib showed similar antitumor activity compared to IgG1-AXL-107-vcMMAE alone (FIG. 28).

LXFA 677 and LXFA 677 3 PDX Models

The anti-tumor activity of IgG1-AXL-107-vcMMAE was evaluated in the subcutaneous NSCLC PDX model LXFA 677 and the LXFA 677_3 model, which is derived from the LXFA 677 model and has acquired resistance to erlotinib (experiments performed by Oncotest, Freiburg, Germany). Inoculation of tumor fragments into 4-6 weeks old male NMRI nu/nu mice and randomization was performed as described above.

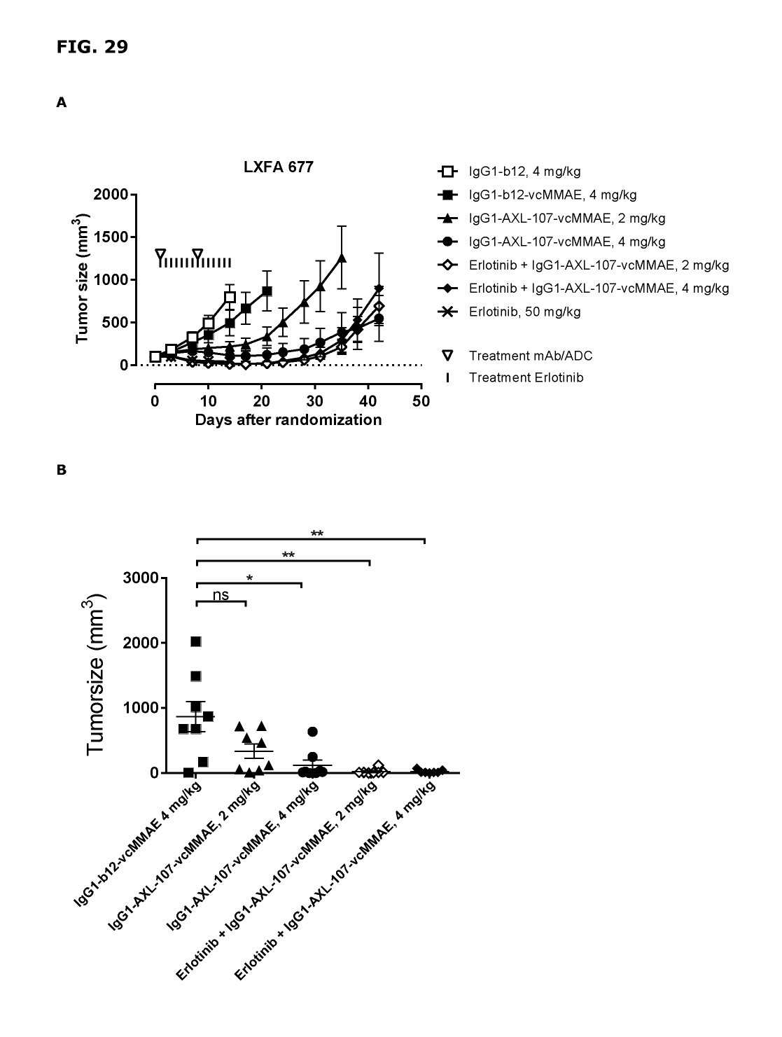

Treatment with IgG1-AXL-107-vcMMAE (2 or 4 mg/kg) was performed at day 0 and 7 after randomization of the groups. IgG1-AXL-107-vcMMAE treatment in combination with the EGFR inhibitor erlotinib was also tested. Erlotinib was given daily for 14 days at a dose of 50 mg/kg. Erlotinib alone, IgG1-b12-vcMMAE and IgG1-b12 were used as controls. Erlotinib induced partial tumor regression in the LXFA 677 model but had no effect on tumor growth in the erlotinib-resistant LXFA 677_3 model, as expected (FIG. 29). IgG1-AXL-107-vcMMAE induced tumor growth inhibition at a dose of 2 mg/kg, while at a dose of 4 mg/kg, IgG1-AXL-107-vcMMAE induced partial tumor regression in the LXFA 677 model. In the erlotinib-resistant LXFA 677_3 model, IgG1-AXL-107-vcMMAE induced complete tumor regression at both dose levels, which lasted at least until day 41. In the two models, combination treatment of IgG1-AXL-107-vcMMAE at 4 mg/kg as well as 2 mg/kg with erlotinib induced similar antitumor activity compared to IgG1-AXL-107-vcMMAE alone (FIG. 29).

### Example 11

### CV1664 PDX Model

The anti-tumor activity of IgG1-AXL-107-vcMMAE was evaluated in the subcutaneous cervical cancer PDX model CV1664 in BALB/c nude mice (experiments performed by CrownBioscience, Changping District, Beijing, China). Inoculation of tumor fragments into BALB/c nude mice and randomization was performed as described in Example 23.

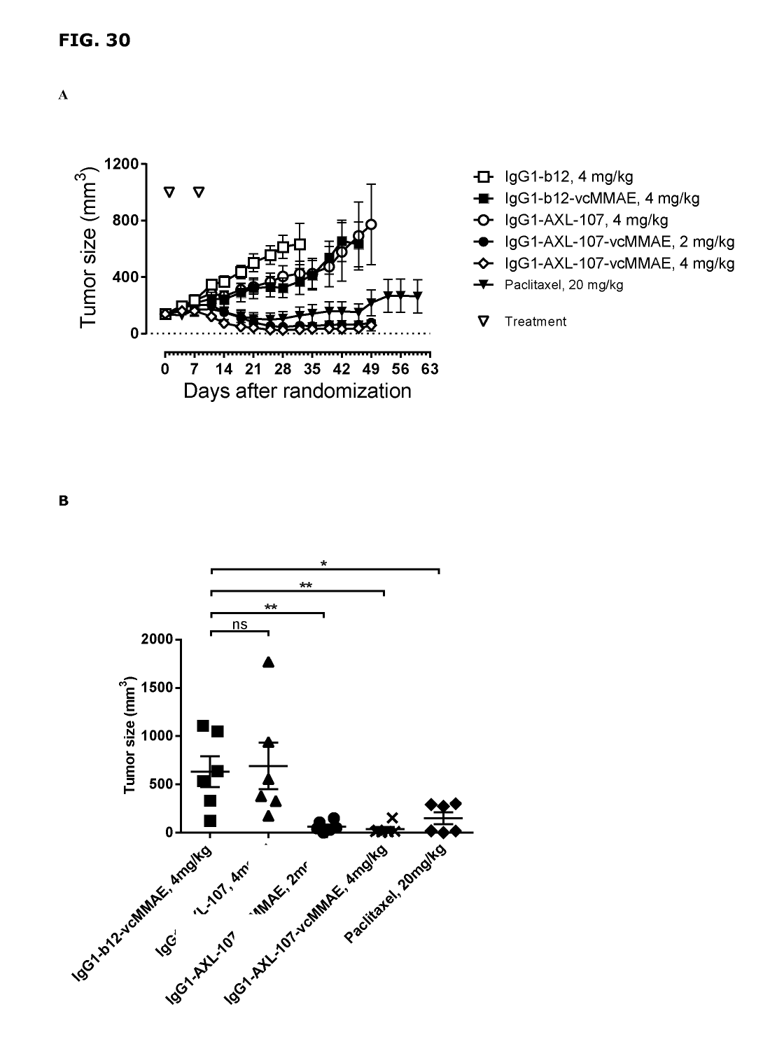

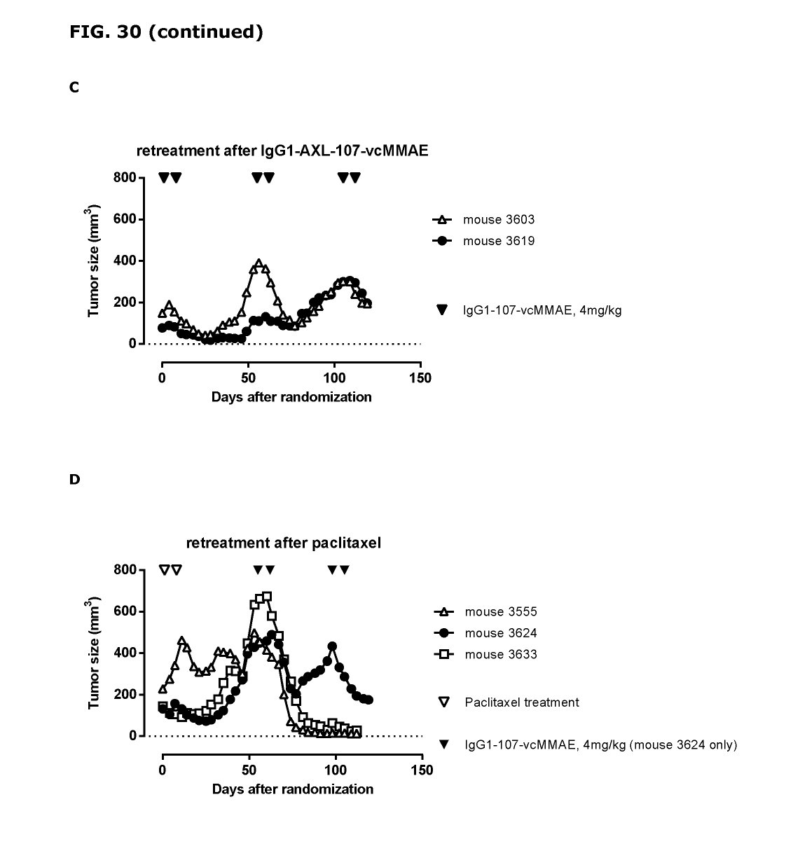

Treatment with IgG1-AXL-107-vcMMAE (2 or 4 mg/kg) was performed at day 0 and 7 after randomization of the groups (FIG. 30). Treatment on the same days with paclitaxel (20 mg/kg; intraperitoneally), unconjugated IgG1-AXL-107 (4 mg/kg), IgG1-b12-vcMMAE (4 mg/kg) and IgG1-b12 (4 mg/kg) were used as controls.

IgG1-AXL-107-vcMMAE induced strong tumor regression at both dose levels, which lasted at least until day 49 (FIG. 30A, B). Treatment with unconjugated IgG1-AXL-107 and IgG1-b12-vcMMAE only induced minor inhibition of tumor growth compared to the IgG1-b12 control group. Paclitaxel induced partial tumor regression.

Two mice that showed tumor regrowth upon initial tumor regression with 4 mg/kg IgG1-AXL-107-vcMMAE were retreated with 2 doses of 4 mg/kg IgG1-AXL-107-vcMMAE on days 55 and 62. This resulted in partial tumor regression in both mice (FIG. 30C). Upon regrowth of the tumors, these mice were retreated again with 2 doses of 4 mg/kg IgG1-AXL-107-vcMMAE on days 105 and 112, which again resulted in partial tumor regression in both animals (FIG. 30C).

Three mice that showed tumor regrowth upon initial tumor regression with paclitaxel were retreated with 2 doses of 4 mg/kg IgG1-AXL-107-vcMMAE on days 55 and 62. Two of the three mice showed complete tumor regression upon retreatment with IgG1-AXL-107-vcMMAE (FIG. 30D). The other mouse showed partial tumor regression. Upon regrowth of the tumor, this mouse was retreated again with 2 doses of 4 mg/kg IgG1-AXL-107-vcMMAE on days 98 and 105, which again resulted in partial tumor regression (FIG. 30D).

### LIST OF REFERENCES

Paccez et al., Int. J. Cancer 134, 1024-1033 (2014)
Sun et al., Ann Oncol. 14(6):898-906 (2003)
Linger et al., Expert Opin Ther Targets. 2010 October;14(10):1073-90
Korshunov et al, Clin Sci (Lond). 2012 April;122(8):361-8.
Meyer et al., Sci Signal. 2013 Aug. 6;6(287):ra66.
Wilson et al., Cancer Res. 2014 Oct. 15;74(20):5878-90.
Zhang et al., Nat Genet. 2012 Jul. 1;44(8):852-60.
Kim et al., Mol Oncol. 2013 December;7(6):1093-102
Müller et al., Nat Commun. 2014 Dec. 15;5:5712
Konieczkowski et al., 2014, Cancer Discov 4: 816-827.
Lefranc M P. et al., Nucleic Acids Research, 27 et al., 209-212, 1999
Brochet X. Nucl. Acids Res. 36, W503-508 (2008)
Needleman and Wunsch, J. Mol. Biol. 48, 444-453 (1970))
Woyke et al (2001) Antimicrob. Agents and Chemother. 45(12): 3580-3584
Pettit et al., (1998) Antimicrob. Agents and Chemother. 42:2961-2965
Kagan et al., Pharm Res (2010) 27:920-932
Dirks and Meibohm (Clin Pharmacokinet (2010) 49 (19):633-659
Gibiansky et al. (J Pharmacokinet Pharmacodyn 2008;35(5):573-91)
Lu et al. (Pharm Res 2015;32:1907-19)
Liu et al., Cancer Res. 2009 Sep. 1;69(17):6871-8
Zhang et al., Nat Genet. 2012 Jul. 1;44(8):852-60
Mahadevan et al., Oncotarget. 2015 Feb. 10;6(4):1954-66
Zhou et al., Oncogene. 2016 May;35(21):2687-97
Debruyne et al., Oncogene. 2015 Nov. 30. doi: 10.1038/onc.2015.434
Park et al., Leukemia. 2015 December;29(12):2382-9
Dufies et al., Oncotarget. 2011 November;2(11):874-85
Elkabets et al., Cancer Cell. 2015 Apr. 13;27(4):533-46
Brand et al., Clin Cancer Res. 2015 Jun. 1;21(11):2601-12
Huang et al., Cancer Res. 2010 Sep. 15;70(18):7221-31.
Müller et al., Nat Commun. 2014 Dec. 15;5:5712
Konieczkowski et al., 2014, Cancer Discov 4: 816-827
Pettazzoni et al., Cancer Research 2015;75: 1091-1101.
Hong et al., Clin Cancer Res. 2012 Apr. 15;18(8):2326-35
Bansal et al., Oncotarget. 2015 Jun. 20;6(17):15321-31
Hong et al., Cancer Lett. 2008 Sep. 18;268(2):314-24.
Brand et al., Clin Cancer Res. 2015 Jun 1;21(11):2601-12
WO 2016/005593 A1 (Genmab)
WO 2017/009258 (Genmab)
WO 2014/174111 A1 (Pierre Fabre Medicament and Spirogen Sarl)
US 2011/0268751 A1 (Seattle Genetics)
U.S. Pat. No. 5,635,483
U.S. Pat. No. 5,780,588
U.S. Pat. No. 5,663,149

1. An antibody drug conjugate (ADC) comprising an antibody binding to human AXL for use in a method of treating a cancer, the method comprising administering the ADC to a subject in need thereof in at least one cycle comprising administration once a week for three consecutive weeks followed by a one week resting period without any administration of ADC so that each cycle time is 28 days including the resting period,
wherein the antibody is conjugated to an auristatin or a functional peptide analog or derivate thereof via a linker, and comprises a variable heavy chain (VH) region and a variable light chain (VL) region selected from the group consisting of
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733]; and
(d) a variant of any of said antibodies defined in (a) to (c), wherein said variant preferably has at most 1, 2 or 3 amino-acid modifications, more preferably amino-acid substitutions, such as conservative amino-acid substitutions across the six CDR sequences.

2. The ADC for use according to claim 1, wherein the auristatin is monomethyl auristatin E (MMAE): 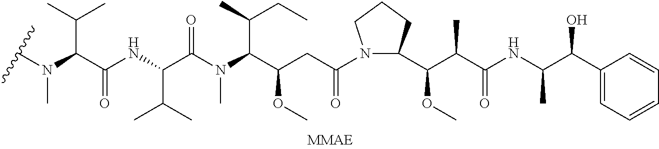 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]
wherein the wavy line indicates the attachment site for the linker.

3. The ADC for use according to any one of the preceding claims wherein the linker-auristatin is vcMMAE: 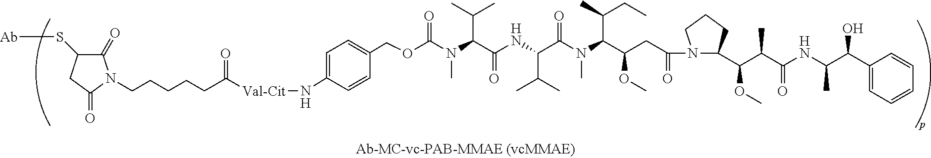
wherein p denotes a number of from 1 to 8, such as from 3-5, preferably 4, S represents a sulphydryl residue of the anti-AXL antibody, and Ab designates the anti-AXL antibody.

4. An ADC of the formula: 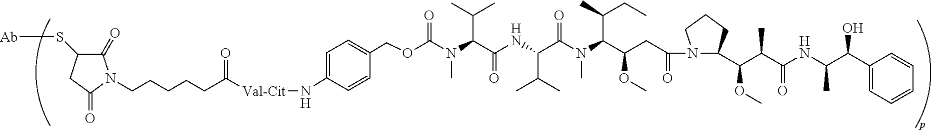
or a pharmaceutically acceptable salt thereof, wherein the antibody binds to human AXL but does not compete with Growth Arrest-Specific 6 (Gash) for binding to human AXL,
S is a sulfur atom of the antibody,
p is a number from 3-5,
for use in a method of treating a cancer wherein the ADC is administered to a subject in need thereof in at least one cycle comprising administration once a week for three consecutive weeks followed by a one week resting period without any administration of the ADC so that each cycle time is 28 days including the resting period.

5. The ADC for use according to claim 4, wherein the antibody comprises a VH region and a VL region selected from the group consisting of
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733]; and
(d) a variant of any of said antibodies defined in (a) to (c), wherein said variant preferably has at most 1, 2 or 3 amino-acid modifications, more preferably amino-acid substitutions, such as conservative amino-acid substitutions across the six CDR sequences.

6. The ADC for use according to any one of the preceding claims, wherein the antibody comprises a VH region and a VL region selected from the group consisting of
(a) a VH region comprising SEQ ID No: 1 and a VL region comprising SEQ ID No: 2 [107];
(b) a VH region comprising SEQ ID No: 5 and a VL region comprising SEQ ID No: 6 [148];
(c) a VH region comprising SEQ ID No: 34 and a VL region comprising SEQ ID No: 35 [733].

7. The ADC for use according to any one of the preceding claims, wherein the antibody comprises a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107].

8. The ADC for use according to any one of the preceding claims, wherein the antibody comprises a VH region comprising SEQ ID No: 1 and a VL region comprising SEQ ID No: 2 [107].

9. The ADC for use according to any one of claims 1 to 6, wherein the antibody comprises a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.:
49, AAS, and 50, respectively, [148].

10. The ADC for use according to claim 9, wherein the antibody comprises a VH region comprising SEQ ID No: 5 and a VL region comprising SEQ ID No: 6 [148].

11. The ADC for use according to any one of claims 1 to 6, wherein the antibody comprises a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733].

12. The ADC for use according to claim 9, wherein the antibody comprises a VH region comprising SEQ ID No: 34 and a VL region comprising SEQ ID No: 35 [733].

13. The ADC for use according to any one of the preceding claims, wherein the linker is attached to sulfhydryl residues of the antibody obtained by (partial) reduction of the antibody.

14. The ADC for use according to any one of the preceding claims, wherein the average p number is 4.

15. The ADC for use according to any one of the preceding claims wherein the ADC is administered on days 1, 8 and 15 in the cycle of 28 days.

16. The ADC for use according to any one of the preceding claims, wherein the dose of ADC is between 0.45 mg/kg and 2.0 mg/kg of the subject's body weight, such as at a dose of 0.45 mg/kg or at a dose of 0.5 mg/kg or at a dose of 0.6 mg/kg or at a dose of 0.7 mg/kg or at a dose of 0.8 mg/kg or at a dose of 0.9 mg/kg or at a dose of 1.0 mg/kg or at a dose of 1.1 mg/kg or at a dose of 1.2 mg/kg or at a dose of 1.3 mg/kg or at a dose of 1.4 mg/kg or at a dose of 1.5 mg/kg or at a dose of 1.6 mg/kg or at a dose of 1.7 mg/kg or at a dose of 1.8 mg/kg or at a dose of 1.9 mg/kg or at a dose of 2.0 mg/kg.

17. The ADC for use according to any one of any one of the preceding claims, wherein the number of cycles of 28 days is between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

18. The ADC for use according to any one of the preceding claims, wherein the ADC is administered for at least four treatment cycles of 28 days, wherein the ADC in each treatment cycle is administered once a week at a dose of 0.45 mg/kg body weight for three consecutive weeks followed by a resting week without any administration of the antibody drug conjugate.

19. The ADC for use according to any one of claims 1-17, wherein the ADC is administered for at least four treatment cycles of 28 days, wherein the ADC in each treatment cycle is administered once a week at a dose of 0.6 mg/kg body weight for three consecutive weeks followed by a resting week without any administration of the antibody drug conjugate.

20. The ADC for use according to any one of claims 1 to 17, wherein the ADC is administered for at least four treatment cycles of 28 days, wherein the ADC in each treatment cycle is administered once a week at a dose of 0.8 mg/kg body weight for three consecutive weeks followed by a resting week without any administration of the antibody drug conjugate.

21. The ADC for use according to any one of claims 1 to 17, wherein the ADC is administered for at least four treatment cycles of 28 days, wherein the ADC in each treatment cycle is administered once a week at a dose of 1.0 mg/kg body weight for three consecutive weeks followed by a resting week without any administration of the antibody drug conjugate.

22. The ADC for use according to any one of claims 1 to 17, wherein the ADC is administered for at least four treatment cycles of 28 days, wherein the ADC in each treatment cycle is administered once a week at a dose of 1.2 mg/kg body weight for three consecutive weeks followed by a resting week without any administration of the antibody drug conjugate.

23. The ADC for use according to any one of claims 1 to 17, wherein the ADC is administered for at least four treatment cycles of 28 days, wherein the ADC in each treatment cycle is administered once a week at a dose of 1.4 mg/kg body weight for three consecutive weeks followed by a resting week without any administration of the antibody drug conjugate.

24. The ADC for use according to any one of claims 1 to 17, wherein the ADC is administered for at least four treatment cycles of 28 days, wherein the ADC in each treatment cycle is administered once a week at a dose of 1.6 mg/kg body weight for three consecutive weeks followed by a resting week without any administration of the antibody drug conjugate.

25. The ADC for use according to any one of claims 1 to 17, wherein the ADC is administered for at least four treatment cycles of 28 days, wherein the ADC in each treatment cycle is administered once a week at a dose of 1.8 mg/kg body weight for three consecutive weeks followed by a resting week without any administration of the antibody drug conjugate.

26. The ADC for use according to any one of claims 1 to 17, wherein the ADC is administered for at least four treatment cycles of 28 days, wherein the ADC in each treatment cycle is administered once a week at a dose of 2.0 mg/kg body weight for three consecutive weeks followed by a resting week without any administration of the antibody drug conjugate

27. The ADC for use according to any one of the preceding claims, wherein the treatment cycles are followed by maintenance therapy.

28. The ADC for use according to claim 27, wherein the maintenance therapy comprises administering the ADC once every three weeks, such as on day 1 of a cycle of 21 days.

29. The ADC for use according to any one of claims 27 and 28, wherein the dose of ADC for the maintenance therapy is between 0.6 mg/kg and 3.2 mg/kg of the subject's body weight, such as at a dose of about 0.6 mg/kg or at a dose of about 0.8 mg/kg or at a dose of about 1.0 mg/kg or at a dose of about 1.2 mg/kg or at a dose of about 1.4 mg/kg or at a dose of about 1.6 mg/kg or at a dose of about 1.8 mg/kg or at a dose of about 2.0 mg/kg or at a dose of about 2.2 mg/kg or at a dose of about 2.4 mg/kg or at a dose of about 2.6 mg/kg or at a dose of about 2.8 mg/kg or at a dose of about 3.0 mg/kg or at a dose of about 3.2 mg/kg.

30. The ADC for use according to any one of claims 27 to 29, wherein the maintenance therapy is administered in cycles of 21 days and the number of cycles are between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

31. The ADC for use according to any one of the preceding claims, wherein the cancer comprises a solid tumor expressing AXL or is an AXL-expressing hematological cancer.

32. The ADC for use according to claim 31, wherein the AXL-expressing hematological cancer is selected from the group consisting of leukemia, such as chronic lymphocytic leukemia, myeloid leukemia, acute myeloid leukemia (AML) and chronic myeloid leukemia, lymphoma such as Non-Hodgkin lymphoma and multiple myeloma.

33. The ADC for use according to claim 31, wherein the cancer comprises a solid tumor expressing AXL and is selected from the group consisting of lung cancer, such as non-small cell lung cancer (NSCLC) and lung squamous cell carcinoma; a gynaecological cancer such as ovarian cancer, endometrial cancer or cervical cancer; thyroid cancer; a skin cancer, such as melanoma; colorectal cancer, such as colorectal carcinoma and colorectal adenocarcinoma; bladder cancer; bone cancer such as chondrosarcoma; breast cancer such as triple-negative breast cancer; cancers of the central nervous system such as glioblastoma, astrocytoma and neuroblastoma; connective tissue cancer; fibroblast cancer; gastric cancer such as gastric carcinoma; head and neck cancer; kidney cancer; liver cancer, such as hepatocellular carcinoma; muscle cancer; neural tissue cancer; pancreatic cancer such as pancreatic ductal carcinoma and pancreatic adenocarcinoma; and soft tissue sarcoma.

34. The ADC for use according to claim 33, wherein the cancer is selected from the group consisting of non-small cell lung cancer (NSCLC), ovarian cancer, endometrial cancer, cervical cancer, thyroid cancer.

35. The ADC for use according to claim 31, wherein the cancer is resistant to at least one therapeutic agent selected from the group consisting of a tyrosine kinase inhibitor, a serine/threonine kinase inhibitor and a chemotherapeutic agent

36. The ADC for use according to claim 35, wherein the tyrosine kinase inhibitor is an EGFR inhibitor and the serine/threonine inhibitor is selected from the group consisting of a BRAF inhibitor and a MEK inhibitor.

37. The ADC for use according to any one of 35 and 36, wherein the cancer is selected from NSCLC, ovarian cancer, cervical cancer, a melanoma, squamous cell carcinoma of the head and neck (SCCHN), a breast cancer, a gastrointestinal stromal tumor (GIST), a renal cancer, a prostate cancer, a neuroblastoma, a pancreatic cancer, an oesophageal cancer, a rhabdomyosarcoma, an acute myeloid leukemia (AML), or a chronic myeloid leukemia (CML).

38. The ADC for use according to claim 37, wherein the cancer is NSCLC resistant to an EGFR inhibitor.

39. The ADC for use according to claim 38, wherein the EGFR inibitor is at least one of erlotinib, gefitinib and afatinib.

40. The ADC for use according to claim 37, wherein the cancer is ovarian cancer resistant to a taxane or a platinum derivative.

41. The ADC for use according to claim 40, wherein the taxane is paclitaxel and the platinum derivative is cisplatin.

42. The ADC for use according to claim 37, wherein the cancer is cervical cancer resistant to a taxane.

43. The ADC for use according to claim 42, wherein the taxane is at least one of paclitaxel and docetaxel.

44. The ADC for use according to claim 37, wherein the cancer is melanoma resistant to a BRAF-inhibitor and/or a MEK-inhibitor.

45. The ADC for use according to claim 44, wherein the BRAF-inhibitor is at least one of vemurafenib and dabrafenib and the MEK-inhibitor is trametinib.

46. The ADC for the use according to any one of claims 35 to 45, wherein the ADC is for use in combination with at least one other anti-cancer agent.

47. The ADC for the use according to claim 46, wherein the at least one other anti-cancer agent comprises at least one therapeutic agent according to any one of claims 35 to 45, wherein the cancer is resistant to the at least one therapeutic agent in the combination.

48. The ADC for use according to any one of the preceding claims, wherein the ADC is comprised in a pharmaceutical composition.

49. The ADC for use according to claim 48, wherein said pharmaceutical composition further comprises a pharmaceutically acceptable carrier.

50. The ADC for use according to any one of claims 48 and 49, wherein the pharmaceutical composition is administered by injection or infusion, preferably as intravenous infusion.

51. A method for treating a cancer in a subject, the method comprising administering to a subject in need thereof an ADC for at least one cycle of treatment comprising administration of the ADC once a week for three consecutive weeks followed by a one week resting period without any administration of the ADC so that each cycle time is 28 days including the resting period, wherein the ADC comprises an antibody binding to human AXL and is conjugated to an auristatin or a functional peptide analog or derivate thereof via a linker.

52. A method of treating a cancer in a subject, the method comprising administering to a subject in need thereof at least one cycle of treatment comprising administration of an ADC once a week for three consecutive weeks followed by a one week resting period without any administration of the ADC so that each cycle time is 28 days including the resting period, wherein the ADC is of the formula: 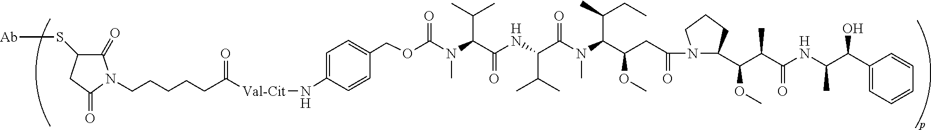
or a pharmaceutically acceptable salt thereof, wherein the Ab is an antibody binding to human AXL,
S is a sulfur atom of the antibody, and
p is a number from 3-5, preferably p is 4.

53. The method of any one of claims 51 and 52 wherein the antibody comprises a VH region and a VL region selected from the group consisting of
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733]; and
(d) a variant of any of said antibodies defined in (a) to (c), wherein said variant preferably has at most 1, 2 or 3 amino-acid modifications, more preferably amino-acid substitutions, such as conservative amino-acid substitutions across the six CDR sequences.

54. The method of any one of claims 51 to 53, comprising the features of the ADC for the use according to any one of claims 1 to 50.


SEQUENCE LISTING (147 sequences)

SEQ ID NO: 1 - protein, 116 aa, homo sapiens
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 2 - protein, 108 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 3 - protein, 120 aa, homo sapiens
EVQLLESGGGLVQPGGSLRLSCAASGFTFSSYAMTWVRQAPGKGLEWVSAISISGASTFYADSVKGRFTISRDNSKNTLSLQMNSLRAEDTAVYFCRGYSGYVYDAFDIWGQGTMVTVSS

SEQ ID NO: 4 - protein, 107 aa, homo sapiens
DIQMTQSPSSLSASVGDRVTITCRASQGISNWLAWYQQKPEKAPKSLIYAASSLQSGVPSRFSGSGSGTDFTLTISSLQPEDFATYYCQQYNSYPLTFGGGTKVEIK

SEQ ID NO: 5 - protein, 120 aa, homo sapiens
EVQLLESGGGLVQPGGSLRLSCAASGFTFSSYAMTWVRQAPGKGLEWVSAISISGGSTFYADSVKGRFTISRDNSKNTLYLQMNSLRAEDTAVYYCRGYSGYVYDAFDFWGQGTMVTVSS

SEQ ID NO: 6 - protein, 107 aa, homo sapiens; identical to SEQ ID NO: 4

SEQ ID NO: 7 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFIMVRGPLDYWGQGALVTVSS

SEQ ID NO: 8 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFILVRGPLDYWGQGALVTVSS

SEQ ID NO: 9 - protein, 108 aa, homo sapiens
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPYTFGQGTKLEIK

SEQ ID NO: 10 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 11 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSFTFGPGTKVDIK

SEQ ID NO: 12 - protein, 125 aa, homo sapiens
EVQLLESGGGLVQPGGSLRLSCAASGFTFSNYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 13 - protein, 107 aa, homo sapiens; identical to SEQ ID NO: 11

SEQ ID NO: 14 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLHMNSLRAEDTAVYYCAKEGYIWFGESLSYAFDIWGQGTMVTVSS

SEQ ID NO: 15 - protein, 107 aa, homo sapiens; identical to SEQ ID NO: 11

SEQ ID NO: 16 - protein, 117 aa, homo sapiens
QVQLQQWGAGLLKPSETLSLTCAVYGGSFSGYYWSWIRQPPGKGLEWIGEINQSGSTNYNPSLKSRVTISVDTSKNQFSLKLSSVTAADTSVYYCASGNWDHFFDYWGQGTLVTVSS

SEQ ID NO: 17 - protein, 117 aa, homo sapiens
QVQLQQWGAGLLKPSETLSLTCAVYGGSFSGYYWSWIRQPPGKGLEWIGEIQQSGSTNYNPSLKSRVTISVDTSKNQFSLKLSSVTAADTSVYYCASGNWDHFFDYWGQGTLVTVSS

SEQ ID NO: 18 - protein, 107 aa, homo sapiens
DIQMTQSPSSVSASVGDRVTITCRASQGISSWLAWYQHKPGKAPKLLIYATSSLQSGVTSRFSGSGSGTDFTLTISSLQPEDFATYYCQQAKSFPWTFGQGTKVEIK

SEQ ID NO: 19 - protein, 123 aa, homo sapiens
QVPLQQWGAGLLKPSETLSLTCAVYGGSFSGYHWSWIRQPPGKGLEWIGEISHSGRTNYNPSLKSRVTISIDTSKNQFSLKLSSVTAADTAVYYCASFITMIRGTIITHFDYWGQGTLVTVSS

SEQ ID NO: 20 - protein, 107 aa, homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYHSYPYTFGQGTKLEIK

SEQ ID NO: 21 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGDYYGSGSPDVFDIWGQGTMVTVSS

SEQ ID NO: 22 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIK

SEQ ID NO: 23 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGNYYGSGSPDVFDIWGQGTMVTVSS

SEQ ID NO: 24 - protein, 107 aa, homo sapiens; identical to SEQ ID NO: 22

SEQ ID NO: 25 - protein, 124 aa, homo sapiens
QVQLVQSGAEVKKPGSSVKVSCKASGGTFSSYAINWMRQAPGQGLEWMGRIIPIFGIVNYAQKFQGRVTLTADKSTSTAYMELSSLRSEDTAVYYCARRGNYYGSGSPDVFDIWGQGTMVTVSS

SEQ ID NO: 26 - protein, 107 aa, homo sapiens; identical to SEQ ID NO: 22

SEQ ID NO: 27 - protein, 124 aa, homo sapiens; identical to SEQ ID NO: 25

SEQ ID NO: 28 - protein, 106 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWLTFGGGTKVEIK

SEQ ID NO: 29 - protein, 124 aa, homo sapiens
QVQLVQSGAEVKKPGSSVKVSCKASGGTFSSYAISWVRQAPGQGLEWMGRIIPIFGIANYAQKFQGRVTITADKSTSTAYMELSSLRSEDTAVYYCARRGNYYGSGSPDVFDIWGQGTMVTVSS

SEQ ID NO: 30 - protein, 107 aa, homo sapiens; identical to SEQ ID NO: 22

SEQ ID NO: 31 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITMIRGAIITHFDYWGQGALVTVSS

SEQ ID NO: 32 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITLIRGAIITHFDYWGQGALVTVSS

SEQ ID NO: 33 - protein, 107 aa, homo sapiens; identical to SEQ ID NO: 20

SEQ ID NO: 34 - protein, 120 aa, homo sapiens
QVQLVESGGGVVQPGRSLRLSCAASGFSFSTYAMHWVRQAPGKGLEWVAVISYDGDNKYSADSVKGRFTISRDNSKNTLYLQMNSLRAEDTAVYYCARGRKLGIDAFDIWGQGTMVTVSS

SEQ ID NO: 35 - protein, 107 aa, homo sapiens
AIQLTQSPSSLSASVGDRVTITCRASQGISSALAWYQQKPGKAPKLLIYDASSLESGVPSRFSGSGSGTDFTLTISGLQPEDFATYYCQQFNSYPFTFGPGTKVDIK

SEQ ID NO: 36 - protein, 8 aa, homo sapiens
GFTFSSYA

SEQ ID NO: 37 - protein, 8 aa, homo sapiens
TSGSGAST

SEQ ID NO: 38 - protein, 9 aa, homo sapiens
AKIWIAFDI

SEQ ID NO: 39 - protein, 7 aa, homo sapiens
QSVSSSY

SEQ ID NO: 40 - protein, 9 aa, homo sapiens
QQYGSSPYT

SEQ ID NO: 41 - protein, 8 aa, homo sapiens
GFTFSSYA

SEQ ID NO: 42 - protein, 8 aa, homo sapiens
ISISGAST

SEQ ID NO: 43 - protein, 13 aa, homo sapiens
RGYSGYVYDAFDI

SEQ ID NO: 44 - protein, 6 aa, homo sapiens
QGISNW

SEQ ID NO: 45 - protein, 9 aa, homo sapiens
QQYNSYPLT

SEQ ID NO: 46 - protein, 8 aa, homo sapiens
GFTFSSYA

SEQ ID NO: 47 - protein, 8 aa, homo sapiens
ISISGGST

SEQ ID NO: 48 - protein, 13 aa, homo sapiens
RGYSGYVYDAFDF

SEQ ID NO: 49 - protein, 6 aa, homo sapiens
QGISNW

SEQ ID NO: 50 - protein, 9 aa, homo sapiens
QQYNSYPLT

SEQ ID NO: 51 - protein, 8 aa, homo sapiens
GFTFSSYA

SEQ ID NO: 52 - protein, 8 aa, homo sapiens
ISIGGGNA

SEQ ID NO: 53 - protein, 14 aa, homo sapiens
AKPGFIMVRGPLDY

SEQ ID NO: 54 - protein, 14 aa, homo sapiens
AKPGFILVRGPLDY

SEQ ID NO: 55 - protein, 7 aa, homo sapiens
QSVSNSY

SEQ ID NO: 56 - protein, 9 aa, homo sapiens
QQYGSSPYT

SEQ ID NO: 57 - protein, 8 aa, homo sapiens
GFTFSSYA

SEQ ID NO: 58 - protein, 8 aa, homo sapiens
ISVSGGST

SEQ ID NO: 59 - protein, 18 aa, homo sapiens
AKEGYIWFGESLSYAFDI

SEQ ID NO: 60 - protein, 7 aa, homo sapiens
QSVSSSY

SEQ ID NO: 61 - protein, 8 aa, homo sapiens
QQYGRSFT

SEQ ID NO: 62 - protein, 8 aa, homo sapiens
GFTFSNYA

SEQ ID NO: 63 - protein, 8 aa, homo sapiens
ISVSGGST

SEQ ID NO: 64 - protein, 18 aa, homo sapiens
AKEGYIWFGESLSYAFDI

SEQ ID NO: 65 - protein, 7 aa, homo sapiens
QSVSSSY

SEQ ID NO: 66 - protein, 8 aa, homo sapiens
QQYGRSFT

SEQ ID NO: 67 - protein, 8 aa, homo sapiens
GFTFSSYA

SEQ ID NO: 68 - protein, 8 aa, homo sapiens
ISVSGGST

SEQ ID NO: 69 - protein, 18 aa, homo sapiens
AKEGYIWFGESLSYAFDI

SEQ ID NO: 70 - protein, 7 aa, homo sapiens
QSVSSSY

SEQ ID NO: 71 - protein, 8 aa, homo sapiens
QQYGRSFT

SEQ ID NO: 72 - protein, 8 aa, homo sapiens
GGSFSGYY

SEQ ID NO: 73 - protein, 7 aa, homo sapiens
INQSGST

SEQ ID NO: 74 - protein, 7 aa, homo sapiens
IQQSGST

SEQ ID NO: 75 - protein, 11 aa, homo sapiens
ASGNWDHFFDY

SEQ ID NO: 76 - protein, 6 aa, homo sapiens
QGISSW

SEQ ID NO: 77 - protein, 9 aa, homo sapiens
QQAKSFPWT

SEQ ID NO: 78 - protein, 8 aa, homo sapiens
GGSFSGYH

SEQ ID NO: 79 - protein, 7 aa, homo sapiens
ISHSGRT

SEQ ID NO: 80 - protein, 17 aa, homo sapiens
ASFITMIRGTIITHFDY

SEQ ID NO: 81 - protein, 6 aa, homo sapiens
QGISSW

SEQ ID NO: 82 - protein, 9 aa, homo sapiens
QQYHSYPYT

SEQ ID NO: 83 - protein, 8 aa, homo sapiens
GGTFSSYA

SEQ ID NO: 84 - protein, 8 aa, homo sapiens
IIPIFGIA

SEQ ID NO: 85 - protein, 17 aa, homo sapiens
ARRGDYYGSGSPDVFDI

SEQ ID NO: 86 - protein, 7 aa, homo sapiens
QSVSSSY

SEQ ID NO: 87 - protein, 8 aa, homo sapiens
QQYGSSYT

SEQ ID NO: 88 - protein, 8 aa, homo sapiens
GGTFSSYA

SEQ ID NO: 89 - protein, 8 aa, homo sapiens
IIPIFGIA

SEQ ID NO: 90 - protein, 17 aa, homo sapiens
ARRGNYYGSGSPDVFDI

SEQ ID NO: 91 - protein, 7 aa, homo sapiens
QSVSSSY

SEQ ID NO: 92 - protein, 8 aa, homo sapiens
QQYGSSYT

SEQ ID NO: 93 - protein, 8 aa, homo sapiens
GGTFSSYA

SEQ ID NO: 94 - protein, 8 aa, homo sapiens
IIPIFGIV

SEQ ID NO: 95 - protein, 17 aa, homo sapiens
ARRGNYYGSGSPDVFDI

SEQ ID NO: 96 - protein, 7 aa, homo sapiens
QSVSSSY

SEQ ID NO: 97 - protein, 8 aa, homo sapiens
QQYGSSYT

SEQ ID NO: 98 - protein, 8 aa, homo sapiens
GGTFSSYA

SEQ ID NO: 99 - protein, 8 aa, homo sapiens
IIPIFGIV

SEQ ID NO: 100 - protein, 17 aa, homo sapiens
ARRGNYYGSGSPDVFDI

SEQ ID NO: 101 - protein, 6 aa, homo sapiens
QSVSSY

SEQ ID NO: 102 - protein, 8 aa, homo sapiens
QQRSNWLT

SEQ ID NO: 103 - protein, 8 aa, homo sapiens
GGTFSSYA

SEQ ID NO: 104 - protein, 8 aa, homo sapiens
IIPIFGIA

SEQ ID NO: 105 - protein, 17 aa, homo sapiens
ARRGNYYGSGSPDVFDI

SEQ ID NO: 106 - protein, 7 aa, homo sapiens
QSVSSSY

SEQ ID NO: 107 - protein, 8 aa, homo sapiens
QQYGSSYT

SEQ ID NO: 108 - protein, 8 aa, homo sapiens
GGSFSGYY

SEQ ID NO: 109 - protein, 7 aa, homo sapiens
ISHSGRT

SEQ ID NO: 110 - protein, 17 aa, homo sapiens
ARFITMIRGAIITHFDY

SEQ ID NO: 111 - protein, 17 aa, homo sapiens
ARFITLIRGAIITHFDY

SEQ ID NO: 112 - protein, 6 aa, homo sapiens
QGISSW

SEQ ID NO: 113 - protein, 9 aa, homo sapiens
QQYHSYPYT

SEQ ID NO: 114 - protein, 8 aa, homo sapiens
GFSFSTYA

SEQ ID NO: 115 - protein, 8 aa, homo sapiens
ISYDGDNK

SEQ ID NO: 116 - protein, 13 aa, homo sapiens
ARGRKLGIDAFDI

SEQ ID NO: 117 - protein, 6 aa, homo sapiens
QGISSA

SEQ ID NO: 118 - protein, 9 aa, homo sapiens
QQFNSYPFT

SEQ ID NO: 119 - protein, 8 aa, homo sapiens
  MISC_FEATURE (6)..(6): Wherein X is A or G
ISISGXST

SEQ ID NO: 120 - protein, 13 aa, homo sapiens
  MISC_FEATURE (13)..(13): Wherein X is I or F
RGYSGYVYDAFDX

SEQ ID NO: 121 - protein, 8 aa, homo sapiens
  MISC_FEATURE (8)..(8): Wherein X is H or y
GGSFSGYX

SEQ ID NO: 122 - protein, 17 aa, homo sapiens
  MISC_FEATURE (2)..(2): Wherein X is S or R
  MISC_FEATURE (10)..(10): Wherein X is T or A
AXFITMIRGXIITHFDY

SEQ ID NO: 123 - protein, 8 aa, homo sapiens
  MISC_FEATURE (6)..(6): Wherein X is S or N
GFTFSXYA

SEQ ID NO: 124 - protein, 8 aa, homo sapiens
ISVSGGST

SEQ ID NO: 125 - protein, 18 aa, homo sapiens
AKEGYIWFGESLSYAFDI

SEQ ID NO: 126 - protein, 8 aa, homo sapiens
  MISC_FEATURE (8)..(8): Wherein X is A or V
IIPIFGIX

SEQ ID NO: 127 - protein, 17 aa, homo sapiens
  MISC_FEATURE (5)..(5): Wherein X is D or N
ARRGXYYGSGSPDVFDI

SEQ ID NO: 128 - protein, 7 aa, homo sapiens
  MISC_FEATURE (4)..(4): Wherein X is S or deleted
QSVXSSY

SEQ ID NO: 129 - protein, 8 aa, homo sapiens
  MISC_FEATURE (3)..(3): Wherein X is R or Y
  MISC_FEATURE (4)..(4): Wherein X is S or G
  MISC_FEATURE (5)..(5): Wherein X is N or S
  MISC_FEATURE (6)..(6): Wherein X is W or S
  MISC_FEATURE (7)..(7): Wherein X is L or Y
QQXXXXXT

SEQ ID NO: 130 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 131 - protein, 904 aa, Mus Musculus
MAWRCPRMGRVPLAWCLALCGWACMYPYDVPDYAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 132-135 - skipped (4 entries)

SEQ ID NO: 136 - protein, 124 aa, homo sapiens
EVQLLESGGGLVQPGGSLRLSCAASGFTFSSYAMNWVRQAPGKGLEWVSGISGSGGHTYHADSVKGRFTISRDNSKNTLYLQMNSLRAEDTAVYYCAKDRYDILTGYYNLLDYWGQGTLVTVSS

SEQ ID NO: 137 - protein, 8 aa, homo sapiens
GFTFSSYA

SEQ ID NO: 138 - protein, 8 aa, homo sapiens
ISGSGGHT

SEQ ID NO: 139 - protein, 17 aa, homo sapiens
AKDRYDILTGYYNLLDY

SEQ ID NO: 140 - protein, 107 aa, homo sapiens
DIQMTQSPSSLSASVGDRVTITCRASQGISSWLAWYQQKPEEAPKSLIYAASSLQSGVPSRFSGSGSGTDFTLTISSLQPEDFATYYCQQYNSYPLTFGGGAKVEIK

SEQ ID NO: 141 - protein, 6 aa, homo sapiens
QGISSW

SEQ ID NO: 142 - protein, 9 aa, homo sapiens
QQYNSYPLT

SEQ ID NO: 143 - protein, 123 aa, homo sapiens
QVQLVQSGAEVKKPGASVKVSCKASGYAFTGYGISWVRQAPGQGLEWIGWISAYNGNTNYVQNLQDRVTMTTDTSTSTAYMELRSLRSDDTAVYYCARDHISMLRGIIIRNYWGQGTLVTVSS

SEQ ID NO: 144 - protein, 108 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSSWPRLTFGGGTKVEIK

SEQ ID NO: 145 - protein, 124 aa, homo sapiens
QVQLVQSGAEVKKPGSSVKVSCKASGGTFSRYAISWVRQAPGQGLEWMGRIIPIVGIANYAQKFQGRVTLTADKSTSTAYMELSSLRSEDTAVYYCAREAGYSSSWYAEYFQHWGQGTLVTVSS

SEQ ID NO: 146 - protein, 108 aa, homo sapiens
EIVLTQSPGTLSLSPGERATLSCRASQSVSSNYLAWYQQKPGQAPRLLIYGASSRATGFPDRFSGSGSGTDFTLTISRLEPEDFAVYYCQQYGSSPYTFGQGTKLEIK

SEQ ID NO: 147 - protein, 893 aa, Macaca fascicularis
AWRCPRMGRVPLAWCLALCGWVCMAPRGTQAEESPFVGNPGNITGARGLTGTLRCQLQVQGEPPEVHWLRDGQILELADSTQTQVPLGEDEQDDWIVVSQLRIASLQLSDAGQYQCLVFLGHQNFVSQPGYVGLEGLPYFLEEPEDRTVAANTPFNLSCQAQGPPEPVDLLWLQDAVPLATAPGHGPQRNLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTLQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKETSAPAFSWPWWYILLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQLDPKDSCSCLTSAEVHPAGRYVLCPSTAPSPAQPADRGSPAAPGQEDGA